(19) United States
(12) Patent Application Publication
(10) Pub. No.: US 2019/0133128 A1
(43) Pub. Date: May 9, 2019

(54) Fungicidal pyridazinones

(71) Applicant: FMC Corporation, Philadelphia, PA (US)

(72) Inventors: Travis Chandler McMahon, Newark, DE (US); Eric Allen Marshall, Rising Sun, MD (US); Thomas Martin Stevenson, Newark, DE (US)

(21) Appl. No.: 15/998,769

(22) Filed: Aug. 16, 2018

Related U.S. Application Data
(60) Provisional application No. 62/546,692, filed on Aug. 17, 2017.

(51) Int. Cl. A01N 43/58; C07D 405/12; C07D 237/24; C07D 401/12; C07D 417/12; A01N 43/82; C07D 403/12

(52) U.S. Cl. CPC A01N 43/58; C07D 405/12; C07D 237/24; C07D 403/12; C07D 417/12; A01N 43/82; C07D 401/12

(57) ABSTRACT

Disclosed are compounds of Formula 1, including all geometric and stereoisomers, N-oxides, and salts thereof,

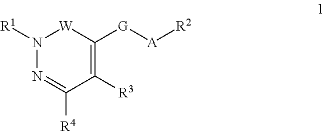

wherein
W, G, A, R¹, R², R³ and R⁴ are as defined in the disclosure.

Also disclosed are compositions containing the compounds of Formula 1 and methods for controlling plant disease caused by a fungal pathogen comprising applying an effective amount of a compound or a composition of the invention.

### CROSS REFERENCE TO RELATED APPLICATIONS

This application claims priority to U.S. Provisional Application Ser. No. 62/546,692, filed Aug. 17, 2017, the content of which is incorporated in its entirety.

### FIELD OF THE INVENTION

This invention relates to certain pyridazinones, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

PCT Patent Publications WO 2006/129432, WO 2007/007903 and WO 2007/007905 disclose pyridinone carboxamide derivatives and their use as fungicides.

### SUMMARY OF THE INVENTION

This invention is directed to compounds of Formula 1 (including all geometric and stereoisomers), including N-oxides, and salts thereof, agricultural compositions containing them and their use as fungicides:

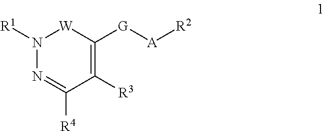

wherein
W is C(═O) or C(═S);
R¹ is H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₄-C₇ cycloalkylalkyl, C₄-C₇ alkylcycloalkyl, C₂-C₆ cyanoalkyl, C₁-C₆ alkoxy, C₁-C₆ haloalkoxy, C₂-C₆ alkoxyalkyl, C₂-C₆ haloalkoxyalkyl, C₃-C₆ alkoxyalkoxyalkyl, C₁-C₆ alkylthio, C₁-C₆ haloalkylthio, C₂-C₄ alkylthioalkyl, C₁-C₆ alkylsulfinyl, C₁-C₆ haloalkylsulfinyl, C₂-C₆ alkylsulfinylalkyl, C₁-C₆ alkylsulfonyl, C₁-C₆ haloalkylsulfonyl, C₂-C₆ alkylsulfonylalkyl, C₁-C₆ alkylsulfonyloxy, C₁-C₆ alkylaminosulfinyl, C₁-C₆ haloalkylaminosulfinyl, C₂-C₆ dialkylaminosulfinyl, C₁-C₆ alkylaminosulfonyl, C₁-C₆ haloalkylaminosulfonyl, C₂-C₆ dialkylaminosulfonyl, C₂-C₆ alkylcarbonyl, C₂-C₆ haloalkylcarbonyl, C₂-C₆ alkoxycarbonyl, C₃-C₆ alkylcarbonylalkyl, C₃-C₆ alkoxycarbonylalkyl, C₂-C₆ alkylaminocarbonyl, C₂-C₆ haloalkylaminocarbonyl, C₃-C₆ dialkylaminocarbonyl or -L(CR^{5a}R^{5b})ₘQ¹;
L is O or a direct bond;
each R^{5a} is independently H, cyano, halogen or C₁-C₄ alkyl;
each R^{5b} is independently H or C₁-C₄ alkyl;
Q¹ is a phenyl ring optionally substituted with up to 5 substituents independently selected from R^{6a}; or a 5- to 6-membered heterocyclic ring, each ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 5 substituents independently selected from R^{6a} on carbon atom ring members and R^{6b} on nitrogen atom ring members;
each R^{6a} is independently halogen, cyano, hydroxy, nitro, amino, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₄-C₆ alkylcycloalkyl, C₄-C₆ cycloalkylalkyl, C₂-C₆ cyanoalkyl, C₁-C₆ alkoxy, C₁-C₆ haloalkoxy, C₃-C₆ cycloalkoxy, C₂-C₆ alkylcarbonyloxy, C₂-C₆ alkylcarbonyl, C₂-C₆ haloalkylcarbonyl, C₃-C₆ alkylcarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylsulfonyloxy, C₁-C₆ haloalkylsulfonyloxy, C₁-C₆ alkylamino, C₂-C₆ dialkylamino, C₂-C₆ alkylcarbonylamino, CH(═O), —NHCH(═O), —SF₅, —SC≡N or —U—V-T;
G is C(═O), C(═S), S(═O), S(═O)₂ or C(═N)-J;
J is NR^{A}R^{B} or —ORS;
R^{A} is H, cyano, CHO, C₂-C₄ alkylcarbonyl, C₁-C₄ alkyl; or phenyl substituted or unsubstituted with halogen or C₁-C₄ alkyl;
R^{B} is H, cyano, CHO, C₁-C₆ alkyl, C₁-C₆ haloalkyl, C₂-C₆ alkenyl, C₃-C₆ alkynyl, C₂-C₈ alkoxyalkyl, C₂-C₈ haloalkoxyalkyl, C₂-C₈ alkylthioalkyl, C₂-C₈ alkylsulfinylalkyl, C₂-C₈ alkylsulfonylalkyl, C₂-C₈ alkylcarbonyl, C₂-C₈ haloalkylcarbonyl, C₄-C₁₀ cycloalkylcarbonyl, C₂-C₈ alkoxycarbonyl, C₂-C₈ haloalkoxycarbonyl, C₄-C₁₀ cycloalkoxycarbonyl, C₂-C₈ alkylaminocarbonyl, C₃-C₁₀ dialkylaminocarbonyl, C₄-C₁₀ cycloalkylaminocarbonyl, C₁-C₆ alkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₃-C₈ cycloalkylsulfinyl, C₁-C₆ alkylsulfonyl, C₁-C₆ haloalkylsulfonyl, C₃-C₈ cycloalkylsulfonyl, C₁-C₆ alkylaminosulfonyl, C₂-C₈ dialkylaminosulfonyl or C₃-C₁₀ trialkylsilyl; or a phenyl ring or a naphthalenyl ring system, each ring or ring system substituted or unsubstituted with up to 5 substituents independently selected from R¹⁶; or a 4- to 7-membered heterocyclic ring, substituted or unsubstituted on ring members with up to 5 substituents independently selected from R¹⁶; or
R^{A} and R^{B} are taken together along with the nitrogen atom to which they are both bonded to form a 4-, 5- or 6-membered ring containing ring members selected from carbon, oxygen, nitrogen and C(═O); or taken together as a 6- to 10-membered bicyclic ring system; or taken together as an 8- to 13-membered tricyclic ring system, each ring or ring system containing ring members selected from carbon, nitrogen and C(═O) and substituted or unsubstituted with halogen, cyano or C₁-C₄ alkyl;
R^{C} is H, CHO, C₁-C₆ alkyl, C₁-C₆ haloalkyl, C₂-C₆ alkenyl, C₃-C₆ alkynyl, C₂-C₈ alkoxyalkyl, C₂-C₈ haloalkoxyalkyl, C₂-C₈ alkylthioalkyl, C₂-C₈ alkylsulfinylalkyl, C₂-C₈ alkylsulfonylalkyl, C₂-C₈ alkylcarbonyl, C₂-C₈ haloalkylcarbonyl, C₄-C₁₀ cycloalkylcarbonyl, C₂-C₈ alkoxycarbonyl, C₂-C₈ haloalkoxycarbonyl, C₄-C₁₀ cycloalkoxycarbonyl, C₂-C₈ alkylaminocarbonyl, C₃-C₁₀ dialkylaminocarbonyl, C₄-C₁₀ cycloalkylaminocarbonyl, C₁-C₆ alkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₃-C₈ cycloalkylsulfinyl, C₁-C₆ alkylsulfonyl, C₁-C₆ haloalkylsulfonyl, C₃-C₈ cycloalkylsulfonyl, C₁-C₆ alkylaminosulfonyl, C₂-C₈ dialkylaminosulfonyl or C₃-C₁₀ trialkylsilyl; or a phenyl ring or a naphthalenyl ring system, each ring or ring system substituted or unsubstituted with up to 5 substituents independently selected from R¹⁶; or a 4- to 7-membered heterocyclic ring, substituted or unsubstituted on ring members with up to 5 substituents independently selected from R¹⁶;
A is N(R^{7a}), N(R^{7b})S(═O)ₙ or S(═O)ₙN(R^{7b});
R² is —(CR^{31a}R^{31b})ᵣQ²;
each R^{31a} is independently H, cyano, halogen or C₁-C₄ alkyl;
each R^{31b} is independently H or C₁-C₄ alkyl;
Q² is a phenyl ring or a naphthalenyl ring system, each ring or ring system optionally substituted with up to 5 substituents independently selected from R^{8a}; or a 5- to 6-membered heterocyclic ring, each ring containing ring members selected from carbon atoms and 1 to 4 heteroatoms independently selected from up to 2 O, up to 2 S and up to 4 N atoms, wherein up to 2 carbon ring members are independently selected from C(═O), C(═S), S(═O) and S(═O)₂, each ring optionally substituted with up to 5 substituents independently selected from R^{8a} on carbon atom ring members and R^{8b} on nitrogen atom ring members; or an 8- to 10-membered bicyclic ring system, each ring or ring system containing ring members selected from carbon atoms and 1 to 5 heteroatoms independently selected from up to 2 O, up to 2 S and up to 5 N atoms, wherein up to 3 carbon ring members are independently selected from C(═O) and C(═S), and the sulfur atom ring members are independently selected from S(═O)ₐ(═NR^{8c})_{b}, each ring or ring system optionally substituted with up to 5 substituents independently selected from R^{8a} on carbon atom ring members and selected from R^{8b} on nitrogen atom ring members;
R³ is H, halogen, OR⁹, OC(═Z)R¹⁰, SH, SR¹¹, S(═O)ᵤR¹¹, OS(═O)ᵤR¹¹, NR³²R³³, OC(═Z)NR¹²R¹³, S(═O)NR¹²R¹³, OP(═O)(R³⁴)₂, C₁-C₄ alkyl, C₁-C₄ haloalkyl, C₂-C₄ alkenyl, C₂-C₄ haloalkenyl, C₁-C₄ alkoxy, C₂-C₄ alkoxyalkyl, C₃-C₆ cycloalkyl or C₄-C₇ cycloalkylalkyl;
R⁴ is H, halogen, cyano, hydroxy, nitro, amino, C₁-C₃ alkyl, C₁-C₃ haloalkyl, C₂-C₄ alkenyl, C₂-C₄ haloalkenyl, C₂-C₄ alkynyl, C₂-C₄ haloalkynyl, C₁-C₃ alkoxy, C₁-C₃ haloalkoxy, C₂-C₄ alkoxyalkyl, C₁-C₃ alkylthio, C₁-C₃ haloalkylthio, C₁-C₃ alkylsulfinyl, C₁-C₃ haloalkylsulfinyl, C₂-C₄ alkylsulfinylalkyl, C₁-C₃ alkylsulfonyl, C₁-C₃ haloalkylsulfonyl, C₂-C₄ alkylsulfonylalkyl, C₂-C₄ alkylcarbonyl, C₂-C₄ haloalkylcarbonyl, C₂-C₄ alkoxycarbonyl, C₃-C₄ alkoxycarbonylalkyl, C₁-C₆ alkylamino, C₂-C₆ dialkylamino, C₂-C₄ alkylaminocarbonyl or C₃-C₄ dialkylaminocarbonyl; or a phenyl ring optionally substituted with up to 5 substituents independently selected from R²⁶;
each R^{8a} is independently halogen, cyano, hydroxy, nitro, amino, —CH(═O), —C(═O)OH, —C(═O)NH₂, —C(R^{11a})═N—O—R^{12a}, —C(R^{11a})═N_R^{12a}, —C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₄-C₆ alkylcycloalkyl, C₄-C₆ cycloalkylalkyl, C₂-C₆ cyanoalkyl, C₁-C₆ alkoxy, C₁-C₆ haloalkoxy, C₃-C₆ cycloalkoxy, C₂-C₆ alkylcarbonyloxy, C₂-C₆ alkylcarbonyl, C₂-C₆ haloalkylcarbonyl, C₃-C₆ alkylcarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylsulfonyloxy, C₁-C₆ haloalkylsulfonyloxy, C₁-C₆ alkylamino, C₂-C₆ dialkylamino, C₂-C₈ alkylaminocarbonyl, C₃-C₁₀ dialkylaminocarbonyl, C₃-C₁₀ dihaloalkylaminocarbonyl; C₄-C₁₀ cycloalkylaminocarbonyl, C₂-C₈ haloalkylcarbonylamino, C₂-C₆ alkylcarbonylamino, —NHCH(═O), —SF₅, —SC≡N or —U—V-T; or a phenyl or phenoxy ring optionally substituted with up to 5 substituents independently selected from R²⁷;
each R^{6b} and R^{8b} is independently cyano, C₁-C₃ alkyl, C₂-C₄ alkenyl, C₂-C₄ alkynyl, C₃-C₆ cycloalkyl, C₁-C₃ alkoxy, C₂-C₄ alkoxyalkyl, C₂-C₄ alkylcarbonyl, C₂-C₄ alkoxycarbonyl, C₂-C₄ alkylaminoalkyl or C₃-C₄ dialkylaminoalkyl,
R^{7a} and R^{7b} are each H, hydroxy, amino, C₂-C₆ alkenyl, C₂-C₆ alkynyl, C₃-C₆ cycloalkyl, C(═O)H, S(═O)ₜR¹⁴, C(═Z)R¹⁵ or OR¹⁶; or C₁-C₆ alkyl or C₁-C₆ haloalkyl, each optionally substituted with up to 2 substituents independently selected from R¹⁷;
each R^{8c} is independently H, cyano, C₂-C₃ alkylcarbonyl or C₂-C₃ haloalkylcarbonyl;
R⁹ is H, CH(═O) or C₃-C₆ cycloalkyl; or C₁-C₆ alkyl, C₂-C₆ alkenyl or C₁-C₆ haloalkyl, each optionally substituted with up to 2 substituents independently selected from R¹⁹ or a phenyl ring optionally substituted with up to 5 substituents independently selected from R²⁸; or
R⁹ and R^{7a} are taken together to form a ring selected from the group consisting

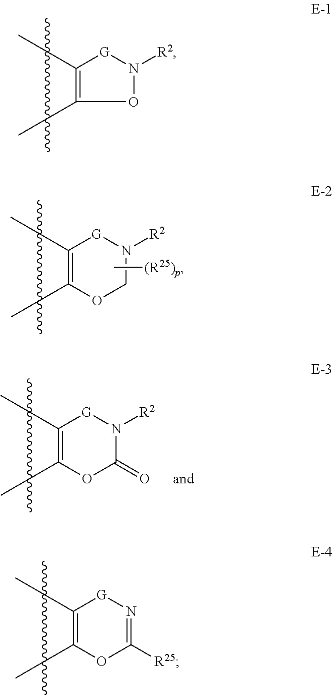

R¹⁰ is C₁-C₆ alkyl, C₁-C₆ haloalkyl, C₂-C₆ alkenyl, C₂-C₆ alkenyl, C₂-C₆ haloalkenyl, C₃-C₆ cycloalkyl, C₁-C₆ alkoxy, C₁-C₆ haloalkoxy, C₂-C₆ alkoxyalkyl, C₁-C₆ alkylthio, C₁-C₆ haloalkylthio, C₂-C₆ alkylthioalkyl, C₂-C₆ alkylaminoalkyl, C₃-C₆ dialkylaminoalkyl; or a phenyl ring optionally substituted with up to 5 substituents independently selected from R²⁸;
R¹¹ is C₁-C₆ alkyl, C₁-C₆ haloalkyl, C₂-C₆ alkenyl, C₂-C₆ alkenyl, C₂-C₆ haloalkenyl, C₃-C₆ cycloalkyl, C₁-C₆ alkoxy, C₁-C₆ haloalkoxy, C₂-C₆ alkoxyalkyl, C₁-C₆ alkylthio, C₁-C₆ haloalkylthio, C₂-C₆ alkylthioalkyl, C₂-C₆ alkylaminoalkyl, C₃-C₆ dialkylaminoalkyl; or phenyl or phenylmethyl (i.e. benzyl), each optionally substituted on ring members with up to 5 substituents independently selected from R²⁹;
each R^{11a} is independently H, C₁-C₃ alkyl, C₁-C₃ haloalkyl or C₁-C₃ cycloalkyl;
each R^{12a} is independently H, C₁-C₃ alkyl, C₁-C₃ haloalkyl, C₂-C₃ alkylcarbonyl or C₂-C₃ haloalkylcarbonyl;
R¹² and R¹³ are each independently H, C₁-C₆ alkyl, C₁-C₆ haloalkyl, C₂-C₆ alkenyl, C₂-C₆ haloalkenyl, C₃-C₆ cycloalkyl, C₂-C₆ alkylcarbonyl, C₂-C₆ haloalkylcarbonyl, C₂-C₆ alkoxycarbonyl or C₂-C₆ haloalkoxycarbonyl;
R¹⁴ is —C₁-C₆ alkyl or C₁-C₆ haloalkyl;
R¹⁵ is C₁-C₆ alkyl, C₁-C₆ haloalkyl, C₁-C₆ alkoxy, C₁-C₆ haloalkoxy, C₂-C₆ alkoxyalkyl, C₁-C₆ alkylthio, C₂-C₆ alkylthioalkyl, C₂-C₆ alkylaminoalkyl, C₃-C₆ dialkylaminoalkyl; or a phenyl ring optionally substituted with up to 5 substituents independently selected from R³⁰;
R¹⁶ is H, CH(═O), C₃-C₆ cycloalkyl or (C═Z)R²⁰; or C₁-C₆ alkyl or C₁-C₆ haloalkyl, each optionally substituted with up to 2 substituents independently selected from R¹⁸;
each R¹⁷, R¹⁸ and R¹⁹ is independently cyano, C₃-C₆ cycloalkyl, C₁-C₆ alkoxy, C₁-C₆ haloalkoxy, C₁-C₆ alkylthio, C₁-C₆ alkylsulfinyl or C₁-C₆ alkylsulfonyl;
R²⁰ are each independently C₁-C₆ alkyl, C₁-C₆ haloalkyl, C₂-C₆ alkenyl, C₂-C₆ haloalkenyl, C₁-C₆ alkoxy, C₁-C₆ haloalkoxy, C₂-C₆ alkoxyalkyl, C₁-C₆ alkylthio, C₂-C₆ alkylthioalkyl, C₂-C₆ alkylaminoalkyl or C₃-C₆ dialkylaminoalkyl,
each U is independently O, S(═O)ₛ, N(R²¹) or a direct bond;
each V is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalkyl, C₁-C₆ alkoxy and C₁-C₆ haloalkoxy;
each T is independently cyano, NR^{22a}R^{22b}, OR²³ or S(═O)ₛR²³;
each R²¹ is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{22a} and R^{22b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{22a} and R^{22b} are taken together with the nitrogen atom to which they are attached to form a form a 3- to 6-membered heterocyclic ring, the ring optionally substituted with up to 5 substituents independently selected from R²⁴;
each R²³ is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, or C₂-C₆ alkoxycarbonyl;
each R²⁴ is independently halogen, C₁-C₆ alkyl, C₁-C₆ haloalkyl or C₁-C₆ alkoxy;
each R²⁵ is independently halogen, hydroxy, amino, cyano, nitro, C₁-C₃ alkyl, C₁-C₃ haloalkyl, C₁-C₂ alkoxy or C₁-C₂ haloalkoxy;
each R²⁶, R²⁷, R²⁸, R²⁹ and R³⁰ is independently halogen, cyano, hydroxy, nitro, amino, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₄-C₆ alkylcycloalkyl, C₄-C₆ cycloalkylalkyl, C₂-C₆ cyanoalkyl, C₁-C₆ alkoxy, C₁-C₆ haloalkoxy, C₃-C₆ cycloalkoxy, C₂-C₆ alkylcarbonyloxy, C₂-C₆ alkylcarbonyl, C₂-C₆ haloalkylcarbonyl, C₃-C₆ alkylcarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylsulfonyloxy, C₁-C₆ haloalkylsulfonyloxy, C₁-C₆ alkylamino, C₂-C₆ dialkylamino, C₂-C₆ alkylcarbonylamino, CH(═O), —NHCH(═O), —SF₅, —SC≡N or —U—V-T;
R³² and R³³ are each independently selected from the group consisting of H, C₁-C₆ alkyl, and C₁-C₆ alkoxy, C₂-C₆ alkylcarbonyl, C₂-C₆ haloalkylcarbonyl and C₂-C₆ alkoxycarbonyl;

each R³⁴ is independently C₁-C₇ alkyl or C₁-C₇ alkoxy;

each a and b are independently 0, 1 or 2 in each instance of S(═O)ₐ(═NR⁸)_{b}, provided that the sum of a and b is 0, 1 or 2;

each m, p, r and s is independently 0, 1 or 2;

each n, t and u is independently 1 or 2; and

Z is O or S.

More particularly, this invention pertains to a compound selected from compounds of Formula 1 (including all stereoisomers) and N-oxides and salts thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of the invention; and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

This invention also relates to a composition comprising a compound of Formula 1, an N-oxide, or a salt thereof, and at least one invertebrate pest control compound or agent.

### DETAILS OF THE INVENTION

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

As referred to herein, the term “broadleaf” used either alone or in words such as “broadleaf crop” means dicot or dicotyledon, a term used to describe a group of angiosperms characterized by embryos having two cotyledons.

As used herein, the term “alkylating agent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating agent” or “alkylating reagent” does not limit the carbon-containing radical to alkyl; the carbon-containing radicals in alkylating agents include the variety of carbon-bound substituent radicals specified.

Generally, when a molecular fragment (i.e. radical) is denoted by a series of atom symbols (e.g., C, H, N, O, S) the implicit point or points of attachment will be easily recognized by those skilled in the art. In some instances, herein, particularly when alternative points of attachment are possible, the point or points of attachment may be explicitly indicated by a hyphen (“-”).

In the above recitations, the term “alkyl”, used either alone or in compound words such as “alkylthio” or “haloalkyl” includes straight-chain or branched alkyl such as methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as 1-propynyl, 2-propynyl and the different butynyl, pentynyl and hexynyl isomers. “Alkynyl” can also include moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃) and the different butylene isomers. “Alkenylene” denotes a straight-chain or branched alkenediyl containing one olefinic bond. Examples of “alkenylene” include CH═CH, CH₂CH═CH and CH═C(CH₃). “Alkynylene” denotes a straight-chain or branched alkynediyl containing one triple bond. Examples of “alkynylene” include C≡C, CH₂C≡C, C≡CCH₂ and the different butynylene isomers.

“Alkylamino” includes an NH radical substituted with straight-chain or branched alkyl. Examples of “alkylamino” include CH₃CH₂NH, CH₃CH₂CH₂NH and (CH₃)₂CHNH. Examples of “dialkylamino” include (CH₃)₂N, (CH₃CH₂)₂N and CH₃CH₂(CH₃)N. “Alkylaminoalkyl” denotes alkylamino substitution on alkyl. Examples of “alkylaminoalkyl” include CH₃NHCH₂, CH₃NHCH₂CH₂ and CH₃CH₂NHCH₂. Examples of “dialkylaminoalkyl” include (CH₃)₂NCH₂, CH₃CH₂(CH₃)NCH₂ and (CH₃)₂NCH₂CH₂.

“Alkoxy” includes, for example, methoxy, ethoxy, n-propyloxy, i-propyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on alkoxy. “Alkenyloxy” includes straight-chain or branched alkenyl attached to and linked through an oxygen atom. Examples of “alkenyloxy” include H₂C═CHCH₂O, (CH₃)₂C═CHCH₂O, CH₃CH═CHCH₂O, CH₃CH═C(CH₃)CH₂O and CH₂═CHCH₂CH₂O. “Alkynyloxy” includes straight-chain or branched alkynyl attached to and linked through an oxygen atom. Examples of “alkynyloxy” include HC≡CCH₂O, CH₃C≡CCH₂O and CH₃C≡CCH₂CH₂O. “Aloxyalkoxyalkyl” denotes alkoxy substitution on the alkoxy moiety of an alkoxyalkyl moiety. Examples of “alkoxyalkoxyalkyl” include CH₃OCH₂OCH₂—, CH₃CH₂O(CH₃)CHOCH₂— and (CH₃O)₂CHOCH₂—.

“Alkylthio” includes branched or straight-chain alkylthio moieties such as methylthio, ethylthio, and the different propylthio isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Alkylsulfinyl” includes both enantiomers of an alkylsulfinyl group. Examples of “alkylsulfinyl” include CH₃S(═O), CH₃CH₂S(═O), CH₃CH₂CH₂S(═O) and (CH₃)₂CHS(═O). Examples of “alkylsulfonyl” include CH₃S(═O)₂, CH₃CH₂S(═O)₂, CH₃CH₂CH₂S(═O)₂ and (CH₃)₂CHS(═O)₂. “Alkylsulfinylalkyl” denotes alkylsulfinyl substitution on alkyl. Examples of “alkylsulfinylalkyl” include CH₃S(═O)CH₂—, CH₃S(═O)CH₂CH₂—, CH₃CH₂S(═O)CH₂— and CH₃CH₂S(═O)CH₂CH₂—. Examples of “alkylsulfonyloxy” include CH₃S(O)₂O—, CH₃CH₂S(O)₂O—, CH₃CH₂CH₂S(O)₂O—, (CH₃)₂CHS(O)₂O—, and the different butylsulfonyloxy, pentylsulfonyloxy and hexylsulfonyloxy isomers. “Cyanoalkyl” denotes an alkyl group substituted with one cyano group. Examples of “cyanoalkyl” include NCCH₂, NCCH₂CH₂ and CH₃CH(CN)CH₂. An example of “alkylaminosulfonyl” is CH₃NHS(O)₂—. An example of “dialkylaminosulfonyl” is (CH₃)₂NS(O)₂—.

The term “cycloalkyl” denotes a saturated carbocyclic ring consisting of between 3 to 7 carbon atoms linked to one another by single bonds. Examples of “cycloalkyl” include cyclopropyl, cyclobutyl, cyclopentyl and cyclohexyl. The term “alkylcycloalkyl” denotes alkyl substitution on a cycloalkyl moiety and includes, for example, methylcyclopropyl, ethylcyclopropyl, i-propylcyclobutyl, 3-methylcyclopentyl and 4-methylcyclohexyl. The term “cycloalkylalkyl” denotes cycloalkyl substitution on an alkyl group. Examples of “cycloalkylalkyl” include cyclopropylmethyl, cyclopentylethyl, and other cycloalkyl moieties bonded to straight-chain or branched alkyl groups. The term “cycloalkoxy” denotes cycloalkyl linked through an oxygen atom such as cyclopentyloxy and cyclohexyloxy. The term “cycloalkylcarbonyl” denotes cycloalkyl substitution bonded through a carbonyl moiety. Examples of “cycloalkylcarbonyl” include c-Pr(C═O)— and cyclopentylC(═O)—. “Cycloalkoxycarbonyl” denotes cycloalkoxy bonded to a C(═O) moiety. Examples of “cycloalkoxycarbonyl” include cyclopentyl-OC(═O)—.

“Alkylcarbonyl” denotes a straight-chain or branched alkyl group bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃OC(═O), CH₃CH₂OC(═O), CH₃CH₂CH₂OC(═O), (CH₃)₂CHOC(═O) and the different pentoxy- or hexoxycarbonyl isomers.

The term “alkylcarbonyloxy” denotes straight-chain or branched alkyl bonded to a C(═O)O moiety. Examples of “alkylcarbonyloxy” include CH₃CH₂C(═O)O and (CH₃)₂CHC(═O)O. “(Alkylthio)carbonyl” denotes a straight-chain or branched alkylthio group bonded to a C(═O) moiety. Examples of “(alkylthio)carbonyl” include CH₃SC(═O), CH₃CH₂CH₂SC(═O) and (CH₃)₂CHSC(═O). “Alkoxy(thiocarbonyl)” denotes a straight-chain or branched alkoxy group bonded to a C(═S) moiety. Examples of “alkylaminocarbonyl” include CH₃NHC(O)—, (CH₃)₂CHNHC(O)— and CH₃CH₂NHC(O)—. Examples of “dialkylaminocarbonyl” include (CH₃)₂NC(═O)—, (CH₃CH₂)₂NC(═O)—, CH₃CH₂(CH₃)NC(═O)—, (CH₃)₂CHN(CH₃)C(═O)— and CH₃CH₂CH₂(CH₃)NC(═O)—. An example of “alkylcarbonylamino” includes NHC(O)CH₃.

The term “halogen”, either alone or in compound words such as “halomethyl”, “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “halocycloalkyl”, “haloalkoxy”, “haloalkylthio”, “haloalkenyl”, “haloalkynyl”, and the like, are defined analogously to the term “haloalkyl”. The term “haloalkoxy” is defined analogously to the term “haloalkyl”. Examples of “haloalkoxy” include CF₃O, CCl₃CH₂O, F₂CHCH₂CH₂O and CF₃CH₂O. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. Examples of “haloalkenyl” include (Cl)₂C═CHCH₂— and CF₃CH₂CH═CHCH₂—. Examples of “haloalkynyl” include HC≡CCHCl—, CF₃C≡C—, CCl₃C≡C— and FCH₂C≡CCH₂—. The term “haloalkylcarbonyl” refers to a haloalkyl group bonded through a carbonyl moiety. Examples of “haloalkylcarbonyl” include CH₂ClC(═O)—, CH₃CHClCH₂C(═O)— and (CH₃)₂CCl(═O)—. The term “haloalkoxyalkyl” refers to a haloalkoxy group bonded through an alkyl moiety. Examples of “haloalkoxyalkyl” include CH₂ClOCH₂—, CHCl₂OCH₂—, CF₃OCH₂—, ClCH₂CH₂OCH₂CH₂—, Cl₃CCH₂OCH₂— as well as branched alkyl derivatives. Examples of “haloalkoxycarbonyl” include CF₃OC(O)—, ClCH₂CH₂OCH₂CH₂—, Cl₃CCH₂OCH₂OC(O)— as well as branched alkyl derivatives.

The total number of carbon atoms in a substituent group is indicated by the “Cᵢ-Cⱼ” prefix where i and j are numbers from 1 to 8. For example, C₁-C₃ alkylsulfonyl designates methylsulfonyl through propylsulfonyl; C₂ alkoxyalkyl designates CH₃OCH₂; C₃ alkoxyalkyl designates, for example, CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The term “unsubstituted” in connection with a group such as a ring means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) range from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 2 substituents independently selected from R¹¹” means that 0, 1 or 2 substituents can be present (if the number of potential connection points allows).

The terms “carbocyclic ring” or “carbocycle” denote a ring wherein the atoms forming the ring backbone are selected only from carbon. When a fully unsaturated carbocyclic ring satisfies Hückel's rule, then said ring is also called an “aromatic carbocyclic ring”. The term “saturated carbocyclic ring” refers to a ring having a backbone consisting of carbon atoms linked to one another by single bonds; unless otherwise specified, the remaining carbon valences are occupied by hydrogen atoms.

R⁹ and R^{7a} can be taken together to form a ring selected from the group consisting

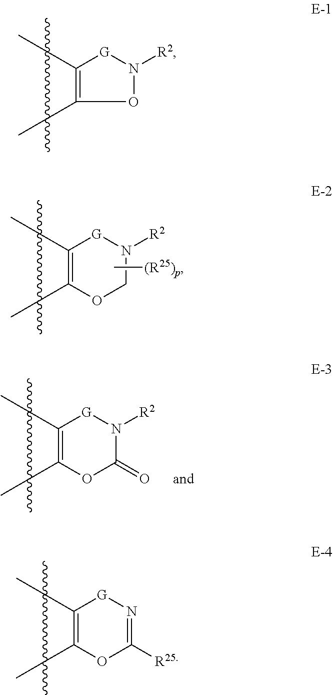

This may also be referred to as taking A and R³ together.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids.

Compounds selected from Formula 1, stereoisomers, N-oxides, and salts thereof, typically exist in more than one form, therefore Formula 1 includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes stereoisomers, N-oxides and salts thereof, and reference to “a compound of Formula 1” includes the definitions of substituents specified in the Summary of the Invention unless further defined in the Embodiments.

### Embodiment 1

A compound of Formula 1 wherein W is C(═O).

### Embodiment 2

A compound of Formula 1 wherein W is C(═S).

### Embodiment 3

A compound of Formula 1 or any one of Embodiments 1 through 2 wherein R¹ is H, C₁-C₆ alkyl or -L(CR^{5a}R^{5b})ₘQ¹.

### Embodiment 4

A compound of Embodiment 3 wherein R¹ is H or C₁-C₃ alkyl.

### Embodiment 5

A compound of Embodiment 4 wherein R¹ is H or CH₃.

### Embodiment 6

A compound of Embodiment 5 wherein R¹ is CH₃.

### Embodiment 7

A compound of Embodiment 3 wherein R¹ is -L(CR^{5a}R^{5b})ₘQ¹.

### Embodiment 8

A compound of Embodiment 7 wherein L is a direct bond.

### Embodiment 9

A compound of Embodiment 7 wherein m is 0.

### Embodiment 10

A compound of Embodiment 7 wherein R^{5a} and R^{5b} are H.

### Embodiment 11

A compound of Embodiment 7 wherein Q¹ is phenyl optionally substituted with 1 substituent selected from R^{6a}.

### Embodiment 12

A compound of Embodiments 11 wherein R^{6a} is C₁-C₆ alkyl or C₁-C₆ haloalkyl.

### Embodiment 13

A compound of Embodiment 12 wherein R^{6a} is CF₃.

### Embodiment 14

A compound of Embodiment 11 wherein Q¹ is unsubstituted phenyl.

### Embodiment 15

A compound of Formula 1 or any one of Embodiments 1 through 14 wherein G is C(═O).

### Embodiment 16

A compound of Formula 1 or any one of Embodiments 1 through 14 wherein G is C(═S).

### Embodiment 17

A compound of Formula 1 or any one of Embodiments 1 through 14 wherein G is S(═O).

### Embodiment 18

A compound of Formula 1 or any one of Embodiments 1 through 14 wherein G is S(═O)₂.

### Embodiment 19

A compound of Formula 1 or any one of Embodiments 1 through 18 wherein A is N(R^{7a}).

### Embodiment 20

A compound of Embodiment 19 wherein R^{7a} is H, C₁-C₆ alkyl or C(═Z)R¹⁵.

### Embodiment 21

A compound of Embodiment 20 wherein R^{7a} is H or C(═Z)R¹⁵.

### Embodiment 22

A compound of Embodiment 20 wherein R^{7a} is C(═Z)R¹⁵.

### Embodiment 23

A compound of Embodiment 22 wherein Z is O.

### Embodiment 24

A compound of Embodiment 23 wherein R¹⁵ is C₁-C₆ alkyl, C₁-C₆ alkoxy or an unsubstituted phenyl ring.

### Embodiment 25

A compound of Embodiment 24 wherein R¹⁵ is C₁-C₆ alkyl.

### Embodiment 26

A compound of Embodiment 20 wherein R^{7a} is H.

### Embodiment 27

A compound of Formula 1 or any one of Embodiments 1 through 26 wherein R² is (CR^{31a}R^{31b})ᵣQ² and Q² is a phenyl ring optionally substituted with up to 3 substituents independently selected from R^{8a}.

### Embodiment 28

A compound of Embodiment 27 wherein Q² is a phenyl ring substituted with up to 2 substituents each independently selected from R^{8a}.

### Embodiment 29

A compound of Embodiment 28 wherein Q² is a phenyl ring substituted with 1 R^{8a} substituent.

### Embodiment 30

A compound of any of Embodiments 27, 28 or 29 wherein each R^{8a} is independently selected from the group consisting of halogen, C₁-C₆ alkyl, C₁-C₆ haloalkyl, cyano, —SF₅, C₁-C₆ alkoxy, C₁-C₆ haloalkoxy, C₁-C₆ haloalkylthio, C₁-C₆ haloalkylsulfonyl, and a phenyl or phenoxy ring optionally substituted with up to 3 substituents independently selected from R²⁷.

### Embodiment 31

A compound of Embodiment 30 wherein each R^{8a} is independently selected from the group consisting of F, Cl, Br, CH₃, CF₃, cyano, —SF₅, OCH₃, OCF₃, OCHF₂, OCF₂CHF₂, —SCF₃, —S(═O)₂CH₃ and a phenyl or phenoxy ring optionally substituted with up to 3 substituents each independently selected from R²⁷.

### Embodiment 32

A compound of Embodiment 31 wherein each R²⁷ is independently selected from halogen.

### Embodiment 33

A compound of Embodiment 32 wherein each R²⁷ is independently selected from the group consisting of F and Cl.

### Embodiment 34

A compound of Embodiment 31 wherein each R^{8a} is independently selected from the group consisting of F, Cl, Br, CF₃, OCH₃ and OCF₃.

### Embodiment 35

A compound of Embodiment 34 wherein R^{8a} is CF₃ or OCF₃.

### Embodiment 36

A compound of Embodiment 35 wherein R^{8a} is CF₃.

### Embodiment 37

A compound of Embodiment 35 wherein R^{8a} is OCF₃.

### Embodiment 38

A compound of Embodiment 27 wherein Q² is unsubstituted phenyl.

### Embodiment 39

A compound of Formula 1 or any one of Embodiments 1 through 26 wherein R² is (CR^{31a}R^{31b})ᵣQ² and Q² is a 5- to 6-membered heterocyclic ring, each ring containing ring members selected from carbon atoms and 1 to 4 heteroatoms independently selected from up to 2 O, up to 2 S and up to 4 N atoms, wherein up to 2 carbon ring members are independently selected from C(═O), C(═S), S(═O) and S(═O)₂, each ring optionally substituted with up to 5 substituents independently selected from R^{8a} on carbon atom ring members and R^{8b} on nitrogen atom ring members.

### Embodiment 40

A compound of Embodiment 39 wherein Q² is selected from the group consisting of a pyridine ring, a pyrimidine ring, a thiadiazole ring and a pyrazole ring, each ring optionally substituted with up to 3 substituents independently selected from R^{8a} on carbon atom ring members.

### Embodiment 41

A compound of Embodiment 40 wherein each R^{8a} is independently selected from the group consisting of halogen, C₁-C₆ alkyl and C₁-C₆ haloalkyl.

### Embodiment 42

A compound of Embodiment 41 wherein each R^{8a} is independently selected from the group consisting of Cl, Br, CH₃ and CF₃.

### Embodiment 43

A compound of Formula 1 or any one of Embodiments 1 through 42 wherein R³ is OR⁹, OC(═Z)R¹⁰, OS(═O)ᵤR¹¹ or halogen.

### Embodiment 44

A compound of Embodiment 43 wherein R³ is OR⁹.

### Embodiment 45

A compound of Embodiment 44 wherein R⁹ is H.

### Embodiment 46

A compound of Embodiment 43 wherein R³ is OC(═Z)R¹⁰.

### Embodiment 47

A compound of Embodiment 46 wherein Z is O and R¹⁰ is C₁-C₆ alkyl, C₁-C₆ haloalkyl, or phenyl optionally substituted with up to 3 substituents each independently selected from R²⁸.

### Embodiment 48

A compound of Embodiment 47 wherein R¹⁰ is selected from the group consisting of C₁-C₆ alkyl, C₁-C₄ alkoxy and unsubstituted phenyl.

### Embodiment 49

A compound of Embodiment 43 wherein R³ is OS(═O)ᵤR¹¹.

### Embodiment 50

A compound of Embodiment 49 wherein u is 2.

### Embodiment 51

A compound of Embodiment 49 wherein R¹¹ is selected from C₁-C₆ alkyl or phenyl optionally substituted with up to 3 substituents each independently selected from R²⁹.

### Embodiment 52

A compound of Embodiment 51 wherein R²⁹ is C₁-C₄ alkyl.

### Embodiment 53

A compound of Formula 1 or any one of Embodiments 1 through 52 wherein R⁴ is H, halogen, C₁-C₆ alkyl, C₁-C₆ alkoxy or phenyl optionally substituted with up to 3 substituents independently selected from R²⁶.

### Embodiment 54

A compound of Embodiment 53 wherein R⁴ is selected from the group consisting of H, Cl, CH₃, OCH₃ and unsubstituted phenyl.

### Embodiment 55

A compound of Embodiment 54 wherein R⁴ is H.

### Embodiment 56

A compound of Formula 1 or any one of Embodiments 1 through 55 wherein
W is C(═O), G is C(═O), A is NH, R¹ is CH₃, R² is phenyl substituted with F, R³ is OH and R⁴ is H; or
   W is C(═O), G is C(═O), A is NH, R¹ is CH₃, R² is phenyl substituted with Cl, R³ is OH and R⁴ is H; or
W is C(═O), G is C(═O), A is NH, R¹ is CH₃, R² is phenyl substituted with Br, R³ is OH and R⁴ is H; or
W is C(═O), G is C(═O), A is NH, R¹ is CH₃, R² is phenyl substituted with CF₃, R³ is OH and R⁴ is H; or
W is C(═O), G is C(═O), A is NH, R¹ is CH₃, R² is phenyl substituted with CHF₂, R³ is OH and R⁴ is H; or
W is C(═O), G is C(═O), A is NH, R¹ is CH₃, R² is phenyl substituted with CH₃, R³ is OH and R⁴ is H; or
W is C(═O), G is C(═O), A is NH, R¹ is CH₃, R² is phenyl substituted with OCH₃, R³ is OH and R⁴ is H; or
W is C(═O), G is C(═O), A is NH, R¹ is CH₃, R² is phenyl substituted with OCF₃, R³ is OH and R⁴ is H; or
W is C(═O), G is C(═O), A is NH, R¹ is CH₃, R² is phenyl substituted with CN, R³ is OH and R⁴ is H; or
W is C(═O), G is C(═O), A is NH, R¹ is CH₃, R² is phenyl substituted with SF₅, R³ is OH and R⁴ is H; or
W is C(═O), G is C(═O), A is NH, R¹ is CH₃, R² is phenyl substituted with OCF₃, R³ is OSO₂Me and R⁴ is H; or
W is C(═O), G is C(═O), A is NH, R¹ is CH₃, R² is phenyl substituted with OCHF₂, R³ is OSO₂Me and R⁴ is H; or
W is C(═O), G is C(═O), A is NH, R¹ is CH₃, R² is phenyl substituted with CN, R³ is OSO₂Me and R⁴ is H; or
W is C(═O), G is C(═O), A is NH, R¹ is CH₃, R² is phenyl substituted with F, R³ is OSO₂Me and R⁴ is H; or
W is C(═O), G is C(═O), A is NH, R¹ is CH₃, R² is phenyl substituted with Br, R³ is OSO₂Me and R⁴ is H; or
W is C(═O), G is C(═O), A is NH, R¹ is CH₃, R² is phenyl substituted with Cl, R³ is OSO₂Me and R⁴ is H; or
W is C(═O), G is C(═O), A is NH, R¹ is CH₃, R² is phenyl substituted with SF₅, R³ is OSO₂Me and R⁴ is H; or
W is C(═O), G is C(═O), A is NH, R¹ is CH₃, R² is phenyl substituted with CF₃, R³ is OSO₂Me and R⁴ is H; or
W is C(═O), G is C(═O), A is NH, R¹ is CH₃, R² is phenyl substituted with OCH₃, R³ is OSO₂Me and R⁴ is H; or
W is C(═O), G is C(═O), A is NH, R¹ is CH₃, R² is phenyl substituted with CH₃, R³ is OSO₂Me and R⁴ is H.

### Embodiment 57

A compound of Formula 1 wherein R³ is OP(═O)(R³⁴)₂ and each R³⁴ is independently C₁-C₇ alkyl or C₁-C₇ alkoxy.

### Embodiment 58

A compound of Formula 1 wherein R⁹ is C₁-C₆ alkyl, C₂-C₆ alkenyl or C₁-C₆ haloalkyl, each optionally substituted with up to 2 substituents independently selected from R¹⁹ or a phenyl ring optionally substituted with up to 5 substituents independently selected from R²⁸.

Embodiments of this invention, including Embodiments 1-58 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-56 above as well as any other embodiments described herein, and any combination thereof, pertain to the compositions and methods of the present invention.

Combinations of Embodiments 1-56 are illustrated by:

### Embodiment A

A compound of Formula 1 wherein
W is C(═O);
R¹ is H, C₁-C₆ alkyl or -L(CR^{5a}R^{5b})ₘQ¹;
L is a direct bond;
R^{5a} and R^{5b} are H;
m is 0;
Q¹ is phenyl optionally substituted with 1 substituent selected from R^{6a};
R^{6a} is C₁-C₆ alkyl or C₁-C₆ haloalkyl;
G is C(═O);
A is N(R^{7a});
R^{7a} is H, C₁-C₆ alkyl or C(═Z)R¹⁵;
Z is O;
R¹⁵ is C₁-C₆ alkyl, C₁-C₆ alkoxy or an unsubstituted phenyl ring;
R² is (CR^{31a}R^{31b})ᵣQ² and Q² is a phenyl ring optionally substituted with up to 3 substituents independently selected from R^{8a};
each R^{8a} is independently selected from the group consisting of halogen, C₁-C₆ alkyl, C₁-C₆ haloalkyl, cyano, —SF₅, C₁-C₆ alkoxy, C₁-C₆ haloalkoxy, C₁-C₆ haloalkylthio, C₁-C₆ halosulfonyl, and a phenyl or phenoxy ring optionally substituted with up to 3 substituents independently selected from R²⁷;
each R²⁷ is independently selected from halogen;
R³ is OR⁹, OC(═Z)R¹⁰, OS(═O)ᵤR¹¹ or halogen;
R¹⁰ is —C₁-C₆ alkyl, C₁-C₆ haloalkyl, or phenyl optionally substituted with up to 3 substituents each independently selected from R²⁸;
R¹¹ is selected from C₁-C₆ alkyl or phenyl optionally substituted with up to 3 substituents each independently selected from R²⁹.
R²⁹ is C₁-C₄ alkyl; and
R⁴ is H, halogen, C₁-C₆ alkyl, C₁-C₆ alkoxy or phenyl optionally substituted with up to 3 substituents independently selected from R²⁶.

### Embodiment B

A compound of Embodiment A wherein
R¹ is H or C₁-C₃ alkyl;
R^{7a} is H or C(═Z)R¹⁵;
R¹⁵ is C₁-C₆ alkyl;
R² is Q² is a phenyl ring substituted with up to 2 substituents each independently selected from R^{8a};
each R^{8a} is independently selected from the group consisting of F, Cl, Br, CH₃, CF₃, cyano, —SF₅, OCH₃, OCF₃, OCHF₂, OCF₂CHF₂, —SCF₃, —S(═O)₂CH₃ and a phenyl or phenoxy ring optionally substituted with up to 3 substituents each independently selected from R²⁷;
each R²⁷ is independently selected from halogen;
R³ is OR⁹; and
R⁴ is selected from the group consisting of H, Cl, CH₃, OCH₃ and unsubstituted phenyl.

### Embodiment C

A compound of Embodiment B wherein
R¹ is H or CH₃;
R^{7a} is H;
each R^{8a} is independently selected from the group consisting of F, Cl, Br, CF₃, OCH₃ and OCF₃;
R³ is OH; and
R⁴ is H.

### Embodiment D

A compound of Embodiment C wherein
R¹ is CH₃;
R² is phenyl, substituted with 1 R^{8a} substituent; and
R^{8a} is CF₃ or OCF₃.

### Embodiment E

A compound of Formula 1 wherein
W is C(═O), G is C(═O), A is NH, R¹ is CH₃, R² is phenyl substituted with F, R³ is OH and R⁴ is H; or
W is C(═O), G is C(═O), A is NH, R¹ is CH₃, R² is phenyl substituted with Cl, R³ is OH and R⁴ is H; or
W is C(═O), G is C(═O), A is NH, R¹ is CH₃, R² is phenyl substituted with Br, R³ is OH and R⁴ is H; or
W is C(═O), G is C(═O), A is NH, R¹ is CH₃, R² is phenyl substituted with CF₃, R³ is OH and R⁴ is H; or
W is C(═O), G is C(═O), A is NH, R¹ is CH₃, R² is phenyl substituted with CHF₂, R³ is OH and R⁴ is H; or
W is C(═O), G is C(═O), A is NH, R¹ is CH₃, R² is phenyl substituted with CH₃, R³ is OH and R⁴ is H; or
W is C(═O), G is C(═O), A is NH, R¹ is CH₃, R² is phenyl substituted with OCH₃, R³ is OH and R⁴ is H; or
W is C(═O), G is C(═O), A is NH, R¹ is CH₃, R² is phenyl substituted with OCF₃, R³ is OH and R⁴ is H; or
W is C(═O), G is C(═O), A is NH, R¹ is CH₃, R² is phenyl substituted with CN, R³ is OH and R⁴ is H; or
W is C(═O), G is C(═O), A is NH, R¹ is CH₃, R² is phenyl substituted with SF₅, R³ is OH and R⁴ is H; or
W is C(═O), G is C(═O), A is NH, R¹ is CH₃, R² is phenyl substituted with OCF₃, R³ is OSO₂Me and R⁴ is H; or
W is C(═O), G is C(═O), A is NH, R¹ is CH₃, R² is phenyl substituted with OCHF₂, R³ is OSO₂Me and R⁴ is H; or
W is C(═O), G is C(═O), A is NH, R¹ is CH₃, R² is phenyl substituted with CN, R³ is OSO₂Me and R⁴ is H; or
W is C(═O), G is C(═O), A is NH, R¹ is CH₃, R² is phenyl substituted with F, R³ is OSO₂Me and R⁴ is H; or
W is C(═O), G is C(═O), A is NH, R¹ is CH₃, R² is phenyl substituted with Br, R³ is OSO₂Me and R⁴ is H; or
W is C(═O), G is C(═O), A is NH, R¹ is CH₃, R² is phenyl substituted with Cl, R³ is OSO₂Me and R⁴ is H; or
W is C(═O), G is C(═O), A is NH, R¹ is CH₃, R² is phenyl substituted with SF₅, R³ is OSO₂Me and R⁴ is H; or
W is C(═O), G is C(═O), A is NH, R¹ is CH₃, R² is phenyl substituted with CF₃, R³ is OSO₂Me and R⁴ is H; or
W is C(═O), G is C(═O), A is NH, R¹ is CH₃, R² is phenyl substituted with OCH₃, R³ is OSO₂Me and R⁴ is H; or
W is C(═O), G is C(═O), A is NH, R¹ is CH₃, R² is phenyl substituted with CH₃, R³ is OSO₂Me and R⁴ is H.

Specific embodiments include compounds of Formula 1 selected from the group consisting of:
2,3-Dihydro-5-hydroxy-2-methyl-3-oxo-N-[4-(trifluoromethoxy)phenyl]-4-pyridazinecarboxamide;
2,3-Dihydro-2-methyl-5-[(methylsulfonyl)oxy]-3-oxo-N-[4-(trifluoromethoxy)phenyl]-4-pyridazinecarboxamide;
2,3-Dihydro-5-hydroxy-2-methyl-3-oxo-N-[4-(trifluoromethyl)phenyl]-4-pyridazinecarboxamide;
N-[4-(Difluoromethoxy)phenyl]-2,3-dihydro-5-hydroxy-2-methyl-3-oxo-4-pyridazinecarboxamide;
N-[2-Fluoro-4-(trifluoromethyl)phenyl]-2,3-dihydro-5-hydroxy-2-methyl-3-oxo-4-pyridazinecarboxamide;
N-(4-Bromophenyl)-2,3-dihydro-5-hydroxy-2-methyl-3-oxo-4-pyridazinecarboxamide;
[4-[[[2,3-Dihydro-5-hydroxy-2-methyl-3-oxo-4-pyridazinyl]carbonyl]amino]phenyl]pentafluorosulfur;
N-[4-(Difluoromethoxy)phenyl]-2,3-dihydro-2-methyl-5-[(methylsulfonyl)oxy]-3-oxo-4-pyridazinecarboxamide;
N-(4-cyanophenyl)-2,3-dihydro-2-methyl-5-[(methylsulfonyl)oxy]-3-oxo-4-pyridazinecarboxamide;
[4-[[[2,3-Dihydro-2-methyl-5-[(methylsulfonyl)oxy]-3-oxo-4-pyridazinyl]carbonyl]amino]phenyl]pentafluorosulfur;
2,3-Dihydro-2-methyl-5-[(methylsulfonyl)oxy]-3-oxo-N-[4-(trifluoromethyl)phenyl]-4-pyridazinecarboxamide;
5-Chloro-2,3-dihydro-2-methyl-3-oxo-N-[4-(trifluoromethoxy)phenyl]-4-pyridazinecarboxamide;
5-Bromo-2,3-dihydro-2-methyl-3-oxo-N-[4-(trifluoromethoxy)phenyl]-4-pyridazinecarboxamide; and
6-Methyl-3-[(trifluoromethoxy)phenyl]-2H-pyridazino[4,5-e]-1,3-oxazine-2,4,5(3H,6H)-trione.

Embodiments of this invention, including Embodiments 1-56 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-56 above as well as any other embodiments described herein, and any combination thereof, pertain to the compositions and methods of the present invention.

One or more of the following methods and variations as described in Schemes 1-23 can be used to prepare the compounds of Formula 1. The definitions of R¹, R², R³, R⁴, A, G, J, and W in the compounds of Formulae 1 through X below are as defined above in the Summary of the Invention unless otherwise noted. The definition of X is halogen and the definition of R^{a} is C₁-C₄ alkyl, unless otherwise noted. Formulae 1a-1l, 4a-4b, 9a-9j, 13a and 18a-18b are various subsets of a compound of Formulae 1, 4, 9, 13 and 18 respectively, unless otherwise noted. Substituents for each subset formula are as defined for its parent formula unless otherwise noted.

As shown in Scheme 1, compounds of Formula 1a (i.e. Formula 1 wherein R³ is C₁-C₄ alkyl, C₂-C₄ alkenyl, C₃-C₆ cycloalkyl, C₄-C₇ cycloalkylalkyl, C₁-C₄ haloalkyl, C₂-C₄ haloalkenyl) can be prepared by the reaction of alkenyl halides of Formula 1b with an organometallic reagent of Formula 2 using well-known metal-catalyzed cross-coupling conditions. The metals employed in these couplings are generally, but not limited to, copper salt complexes (e.g. CuI with N,N′-dimethylethylenediamine, proline or bipyridyl), palladium complexes (e.g., tris(dibenzylideneacetone)dipalladium(0)) or palladium salts (e.g., palladium acetate) with ligands such as 4,5-bis(diphenylphosphino)-9,9-dimethylxanthene, 2-dicyclohexyl-phosphino-2′,4′,6′-triisopropylbiphenyl or 2,2′-bis(diphenylphosphino)1,1′-binaphthalene. One skilled in the art will recognize that a wide variety of organometallic transmetalating groups can be employed in this reaction and, depending on the group, the conditions required to perform the reaction can differ. For example, couplings employing boron reagents require the addition of a suitable base, such as sodium tert-butoxide, potassium carbonate, cesium carbonate or potassium phosphate, whereas in the coupling with tin reagents, the addition of a base is not necessary. Generally, these reactions are run in solvents such as N,N-dimethylformamide, 1,2-dimethoxyethane, dimethyl sulfoxide, 1,4-dioxane or toluene, and at temperatures ranging from room temperature to the boiling point of the solvent employed; typically, 23 to 110° C. For relevant examples of couplings employing structures related to 1b and aluminum reagents, see: J. Heterocyclic Chem. 2005, 42, 427-435; boron reagents, see: J. Organomet. Chem. 2002, 663, 46-57; tin reagents, see: Tetrahedron 2004, 60, 12177-12189.

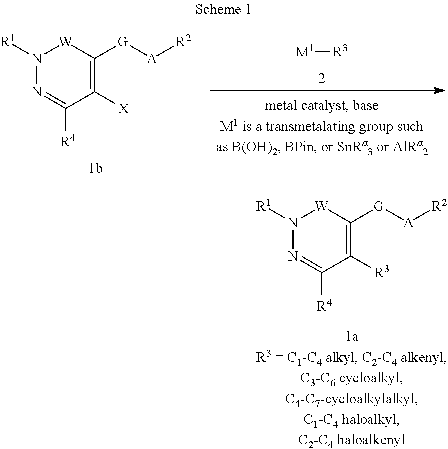

As shown in Scheme 2, compounds of Formulae 1c and 1d (i.e. Formula 1 wherein R³ is S(═O)R¹¹ or S(═O)₂R¹¹, respectively) can be prepared by the reaction of thioethers of Formula 1e with an oxidant. Typical oxidants used in these reactions are peroxides such as meta-chloroperoxybenzoic acid, hydrogen peroxide or tert-butyl hydroperoxide or an oxaziridine. One skilled in the art will recognize that the ratio of 1c and 1d can generally be controlled by the stoichiometry of the oxidant employed. Normally, these reactions are run in a solvent such as dichloromethane, dichloroethane, or acetonitrile at temperatures ranging from 0 to 80° C.

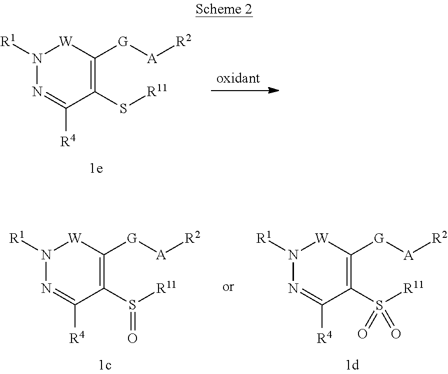

As shown in Scheme 3, thioethers of Formula 1e (i.e. Formula 1 wherein R³ is SR¹¹) can be prepared by the reaction of alkenyl halides of Formula 1b with metal thiolates of Formula 3. These reactions are typically run at temperatures ranging from 0 to 80° C. in a solvent such as N,N-dimethylformamide, dimethylsulfoxide, tetrahydrofuran or acetonitrile utilizing metal thiolates with a counterion such as sodium or potassium.

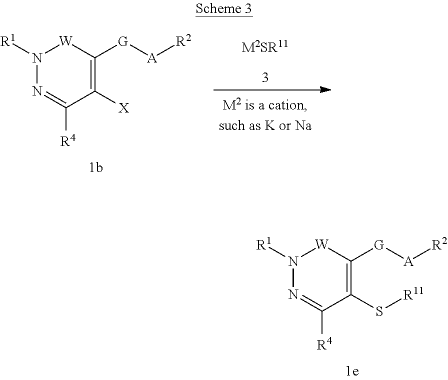

As illustrated in Scheme 4, sulfinamides or sulfonamides of Formula 1f (i.e. Formula 1 wherein R³ is S(═O)ᵤNR¹²R¹³) can be prepared by the reaction of a sulfinic acid or sulfonic acid of Formula 4 with an amine of Formula 5 in the presence of an activating agent. Typical activating agents for these reactions are thionyl chloride, oxalyl chloride, phosphorus pentachloride or phosphorus oxychloride. One skilled in the art will recognize that often it is advantageous to add an additive such as N,N-dimethylformamide in combination with the activating agent. Generally, these reactions are run at temperatures ranging from 0 to 80° C. in a solvent such as dichloromethane, toluene, tetrahydrofuran or acetonitrile.

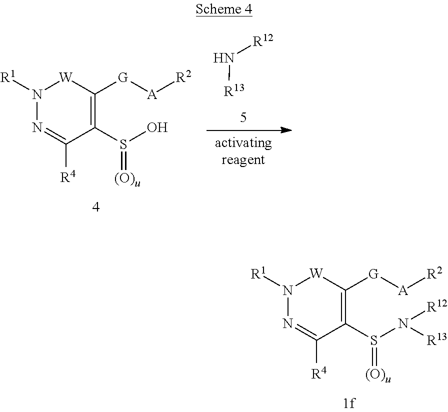

As shown in Scheme 5, sulfinic acids of Formula 4a (i.e. Formula 4 wherein u is 1) can be prepared by the reaction of alkenyl halides of Formula 1b with a metalating reagent and an electrophile such as sulfur dioxide or 1,4-diazabicyclo[2.2.2]octane bis(sulfur dioxide) adduct. Metalating reagents used in this reactions can be, but are not limited to, lithiating reagents such as n-butyl lithium or tert-butyl lithium or magnesiating reagents, such as magnesium metal or isopropylmagnesium chloride lithium chloride complex. These reactions are typically run in solvents such as tetrahydrofuran, diethyl ether, or 1,4-dioxane at temperatures ranging from 78 to 25° C.

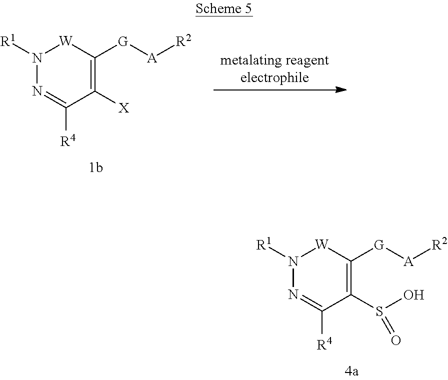

As illustrated in Scheme 6, sulfonic acids of Formula 4b (i.e. Formula 4 wherein u is 2) can be prepared by the reaction of alkenyl halides of Formula 1b with a metal sulfite of Formula 6, such as potassium sulfite, sodium sulfite, potassium bisulfite or sodium bisulfite and optionally in the presence of an additive such as copper(I) chloride or magnesium oxide. These reactions are generally run in water, ethanol or N,N-dimethylformamide at temperatures ranging from room temperature to the boiling point of the solvent, and typically at the boiling point of the solvent or above in a sealed vessel.

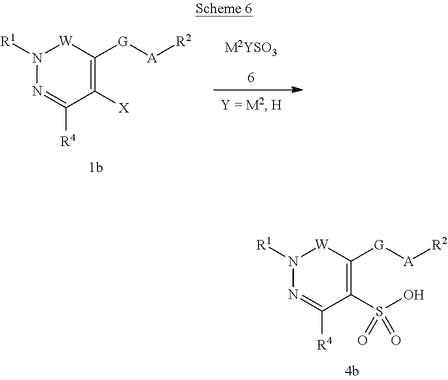

As shown in Scheme 7, compounds of Formula 1g (i.e. Formula 1 wherein R⁹ and R^{7a} are taken together to form E-1) can be prepared from the reaction of Formula 1f (i.e. Formula 1 wherein A is N—OH and R³ is a halogen) with a base. Suitable bases for this reaction can be, but are not limited to, sodium hydride, potassium carbonate, cesium carbonate or triethyl amine. Generally these reactions are run at temperatures ranging from 20 to 80° C. in a solvent such as N,N-dimethylformamide, dichloromethane, acetonitrile or tetrahydrofuran.

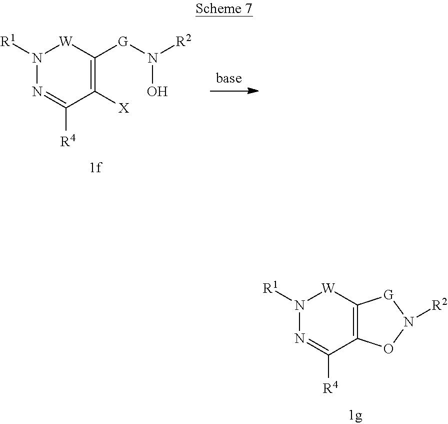

As shown in Scheme 8 compounds of Formula 1b (i.e. Formula 1 wherein R³ is halogen) can be prepared by reaction of alcohols of Formula 1h (i.e. Formula 1 wherein R³ is OH) with halogenating reagents such as oxalyl chloride, oxalyl bromide, phosphorus oxychloride or phosphorus oxybromide. One skilled in the art will recognize that when reagents such as oxalyl chloride or oxlyl bromide are used in this reaction, it is required to add an additional activating reagent such as N,N-dimethylformamide. These reactions are typically run at temperatures ranging from 0-110° C. in a solvent such as dichloromethane, acetonitrile or toluene. The method of Scheme 8 utilizing oxalyl chloride is illustrated in Synthesis Example 3 and the method of Scheme 8 utilizing oxalyl bromide is illustrated in Synthesis Example 4.

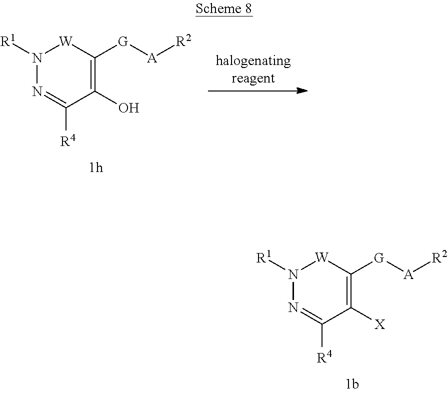

As Shown in Scheme 9, compounds of Formula 1i (i.e. Formula 1 wherein R³ is OR⁹, OC(═Z)R¹⁰, OS(═O)R¹¹, O(C═Z)NR¹²R¹³, op(=O)(R³⁴)₂, OC₂—C₄ alkoxyalkyl, OC₁—C₄ alkyl, or OC₁—C₄ haloalkyl) can be prepared by reaction of alcohols of Formula 1h with alkylating, alkenylating, acylating, phosphorylating or sulfonating reagents of Formula 7. These reactions are typically run at reaction temperatures ranging from 0-80° C. in a solvent such as acetonitrile, tetrahydrofuran, dichloromethane or N,N-dimethylformamide in the presence of a base such as, but not limited to, triethylamine or potassium carbonate. The method of Scheme 9 utilizing methanesulfonyl chloride is illustrated in Synthesis Example 5.

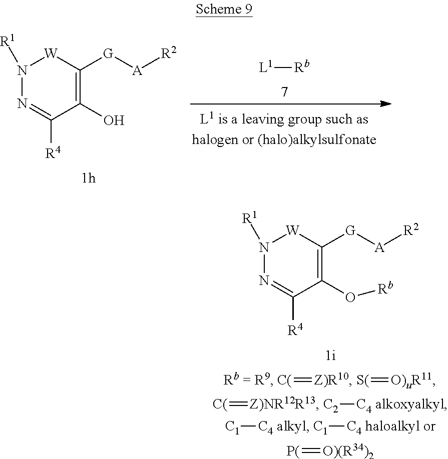

As illustrated in Scheme 10, compounds of Formulae 1j and 1k (i.e. Formula 1 wherein R⁹ and R^{7a} are taken together to form E-2 and E-3, respectively) can be prepared from the reaction of Formula 1h with either an acylating reagent of Formula 8 to form Formula 1j, or an alkylating reagent to form Formula 1k. Typical examples of acylating reagents of Formula 8 are phosgene, triphosgene and cabonyldiimidazole. Generally, the alkylating reagents used to form compounds of Formula 1k are alkyl-1,1-dihalides. Typically, these reactions are run in the presence of a base, such as triethylamine, potassium carbonate or cesium carbonate in a solvent such as N,N-dimethylformamide, dichloromethane, acetonitrile or tetrahydrofuran at temperatures ranging from 0-80° C. Alternatively, carbonyl compounds, such as formaldehyde in the case wherein R²⁵ is H, can be employed to form compounds of Formula k. One skilled in the art will recognize that the reaction with a carbonyl compound will generally be run in the presence of an acid, such as para-toluenesulfonic acid or hydrochloric acid and can be aided by the addition of a dehydrating reagent such as magnesium sulfate or sodium sulfate or run in a Dean-Stark apparatus to remove water.

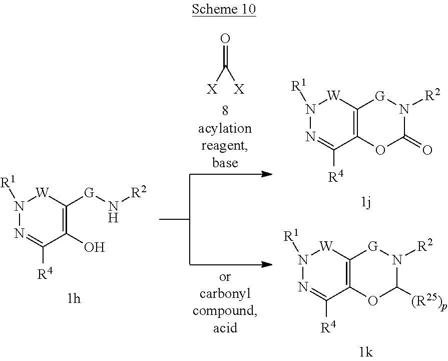

As shown in Scheme 11, compounds of Formula 1h can be prepared by the reaction of compounds of Formula 9 with either a hydroxide base or a Lewis acid. The hydroxide bases used in this reaction typically have a counterion such as sodium, potassium or tetrabutylammonium and are run in solvents such as methanol, ethanol or tetrahydrofuran, generally with water as a cosolvent at temperatures ranging from room temperature to the boiling point of the solvent. Alternatively, the Lewis acids used in this reaction are usually, but not limited to, boron trichloride or boron tribromide. These reactions are generally run in a solvent such as dichloromethane, dichloroethane or toluene at temperatures ranging from 0 to 110° C.

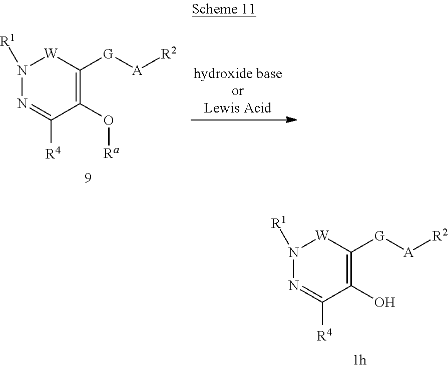

As illustrated in Scheme 12, compounds of Formula 1l (i.e. Formula 1 wherein R⁹ and R^{7a} are taken together to form E-4) can be prepared by the reaction of compounds of Formula 10 with orthoesters of Formula 11 or cyanides of Formula 12. Generally, these reactions are run in the presence of a Lewis or Brønsted acid such as para-toluenesulfonic acid, hydrochloric acid or zinc chloride in a solvent such as toluene, N,N-dimethylformamide, benzene, or dichloromethane at a temperature ranging from room temperature to the boiling point of the solvent.

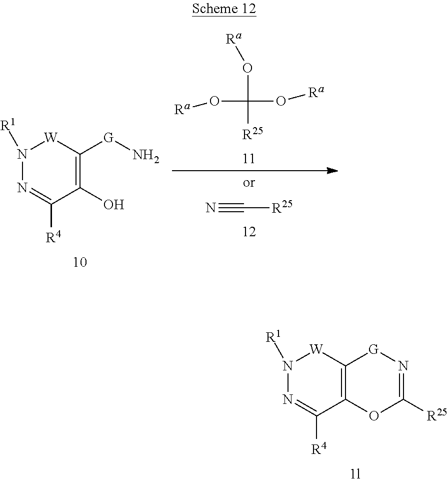

As shown in Scheme 13, compounds of Formula 10 can be prepared by reaction of compounds of Formula 13a (i.e. Formula 13 wherein R^{7b} is H) with either a hydroxide base or a Lewis acid. The hydroxide bases used in this reaction typically have a counterion such as sodium, potassium or tetrabutylammonium and are run in solvents such as methanol, ethanol or tetrahydrofuran, generally with water as a cosolvent at temperatures ranging from room temperature to the boiling point of the solvent. Alternatively, the Lewis acids used in this reaction are usually, but not limited to, boron trichloride or boron tribromide. These reactions are generally run in a solvent such as dichloromethane, dichloroethane or toluene at temperatures ranging from 0 to 110° C.

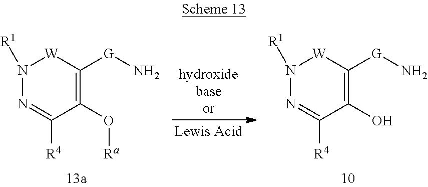

As shown in Scheme 14, compounds of Formula 9a (i.e. Formula 9 wherein G is C═N-J) can be prepared by the reaction of thiocarbonyls of Formula 9b with amines of Formula 14. These reactions can be aided by the addition of an activating agent such as a Lewis or Brønsted acid and are typically run at temperatures ranging from 0-120° C. in a solvent such as methanol, ethanol, tetrahydrofuran, acetonitrile, dichloromethane or N,N-dimethylformamide. Alternatively, the sulfur can be activated by first alkylating with an alkyl halide or alkyl (halo)alkylsulfonate, such as methyl iodide or methyl triflate.

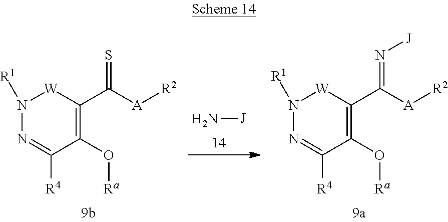

As shown in Scheme 15, compounds of Formula 9b (i.e. Formula 9 wherein G is C═S) can be prepared by the reaction of carbonyls of Formula 9c (i.e. Formula 9 wherein G is C═O) with a thionation reagent such as Lawesson's reagent, tetraphosphorus decasulfide or diphosphorus pentasulfide in a solvent such as tetrahydrofuran or toluene. Typically, the reaction is carried out at temperatures ranging from 0 to 115° C.

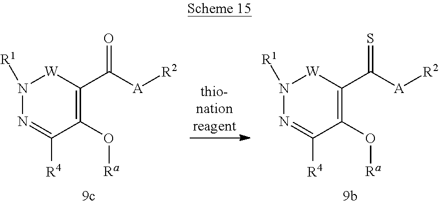

As illustrated in Scheme 16, compounds of Formula 9d (i.e. Formula 9 wherein G is C═O, S═O or S(═O)₂, A is N—R^{7a} and R^{7a} is C(═O)H, S(═O)ₜ or C(═Z)R¹⁵) can be prepared by the reaction of compounds of Formula 9e (i.e. Formula 9 wherein G is C═O, S═O or S(═O)₂ and A is N—H) with an acylating or sulfonating reagent. These reactions are typically run in the presence of a base such as triethylamine, potassium carbonate, cesium carbonate or sodium hydride in a solvent such as dichloromethane, N,N-dimethylformamide, acetonitrile or tetrahydrofuran at a temperature ranging from 0° C. to the boiling point of the solvent.

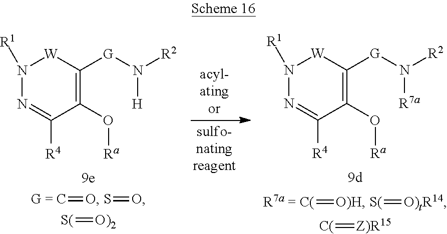

As shown in Scheme 17, compounds of Formula 9f (i.e. Formula 9 wherein G is C═O, S═O or S(═O)₂, A is N—R^{7a} and R^{7a} is H, OH, OR¹⁶, NH₂, C₂-C₆ alkenyl, C₂-C₆ alkynyl, C₃-C₆ cycloalkyl, C₁-C₆ alkyl or C₁-C₆ haloalkyl) can be prepared by the reaction of compounds of Formula 15 with amines of Formula 16 in the presence of a dehydrative coupling reagent such as propylphosphonic anhydride, dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide. Polymer-supported reagents, such as polymer-supported cyclohexylcarbodiimide, are also suitable. These reactions are typically run at temperatures ranging from 0-60° C. in a solvent such as tetrahydrofuran, dichloromethane, acetonitrile, N,N-dimethylformamide or ethyl acetate in the presence of a base such as triethylamine, N,N-diisoprop ylamine, or 1,8-diazabicyclo[5.4.0]undec-7-ene. See Organic Process Research & Development 2009, 13, 900-906 for coupling conditions employing propylphosphonic anhydride. The method of Scheme 17 utilizing propylphosphonic anhydride is illustrated by Step B of Synthesis Example 1.

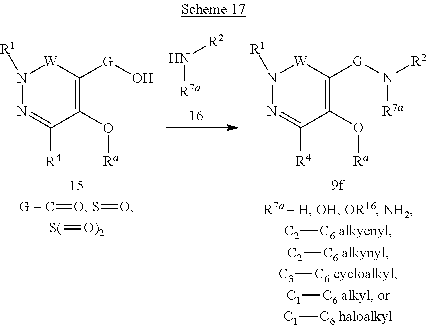

As shown in Scheme 18, compounds of Formula 9g (i.e. Formula 9 wherein A is N(R⁷⁶)S(═O)ₙ and R^{7b} is C(═O)H, S(═O)ₜR¹⁴, or C(═Z)R¹⁵) can be prepared by the reaction of compounds of Formula 9h with an acylating or sulfonating reagent. These reactions are typically run in the presence of a base such as triethylamine, potassium carbonate, cesium carbonate or sodium hydride in a solvent such as dichloromethane, N,N-dimethylformamide, acetonitrile or tetrahydrofuran at a temperature ranging from 0° C. to the boiling point of the solvent.

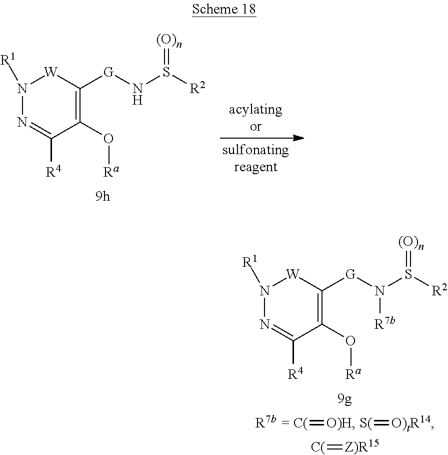

As shown in Scheme 19, compounds of Formula 9h (i.e. Formula 9 wherein A is N(R⁷⁶)S(═O)ₙ and R^{7b} is H, OH, OR¹⁶, NH₂, C₂-C₆ alkenyl, C₂-C₆ alkynyl, C₃-C₆ cycloalkyl, C₁-C₆ alkyl or C₁-C₆ haloalkyl) can be prepared by the reaction of compounds of Formula 13 with a sulfonating reagent. These reactions are typically run in the presence of a base such as triethylamine, potassium carbonate, cesium carbonate or sodium hydride in a solvent such as dichloromethane, N,N-dimethylformamide, acetonitrile or tetrahydrofuran at a temperature ranging from 0° C. to the boiling point of the solvent.

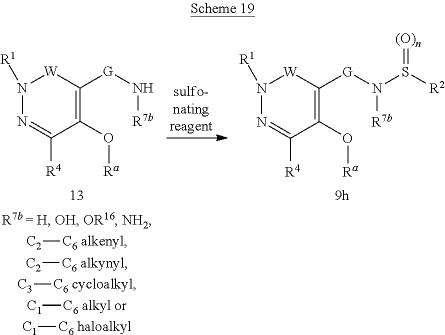

As illustrated in Scheme 20, compounds of Formula 13 can be prepared by the reaction of compounds of Formula 15 with amines of Formula 17 in the presence of a dehydrative coupling reagent such as propylphosphonic anhydride, dicyclohexylcarbodiimide, N-(3-dimethylaminoprop yl)-N′-ethylcarbodiimide, N,N′-carbonyldiimidazole, 2-chloro-1,3-dimethylimidazolium chloride or 2-chloro-1-methylpyridinium iodide. Polymer-supported reagents, such as polymer-supported cyclohexylcarbodiimide, are also suitable. These reactions are typically run at temperatures ranging from 0-60° C. in a solvent such as tetrahydrofuran, dichloromethane, acetonitrile, N,N-dimethylformamide or ethyl acetate in the presence of a base such as triethylamine, N,N-diisopropylamine, or 1,8-diazabicyclo[5.4.0]undec-7-ene. See Organic Process Research & Development 2009, 13, 900-906 for coupling conditions employing propylphosphonic anhydride.

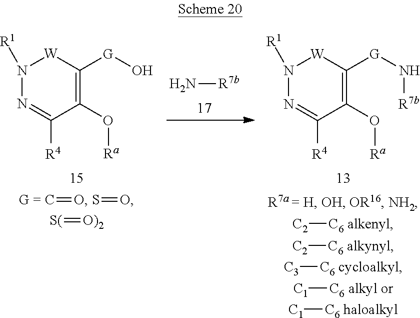

As shown in Scheme 21, compounds of Formula 15 can be prepared from the reaction of compounds of Formula 18 with a base and an electrophile. Typical bases for this reaction include n-butyl lithium, 2,2,6,6-tetramethylpiperidinylmagnesium chloride lithium chloride complex, or bis(2,2,6,6-tetramethylpiperidinyl)zinc. Electrophiles employed in this reactions can be, but are not limited to, carbon dioxide or sulfur dioxide. These reactions are usually run in a solvent such as tetrahydrofuran, diethyl ether, hexanes, or 1,4-dioxane at temperatures ranging from 78 to 25° C. For examples of this reaction in the literature, see: J. Org. Chem. 2009, 74, 9440-9445 and J. Org. Chem. 2011, 76, 6670-6677. The method of Scheme 21 utilizing 2,2,6,6-tetramethylpiperidinylmagnesium chloride lithium chloride complex and carbon dioxide is illustrated by Step A of Synthesis Example 1.

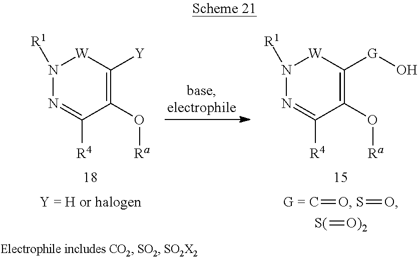

As shown in Scheme 22, amides of Formula 9i can be prepared by the reaction of compounds of Formula 18 with a base and an isocyante of Formula 19. Typical bases for this reaction include n-butyl lithium, 2,2,6,6-tetramethylpiperidinylmagnesium chloride lithium chloride complex, or bis(2,2,6,6-tetramethylpiperidinyl)zinc. These reactions are usually run in a solvent such as tetrahydrofuran, diethyl ether, hexanes, or 1,4-dioxane at temperatures ranging from 78 to 25° C.

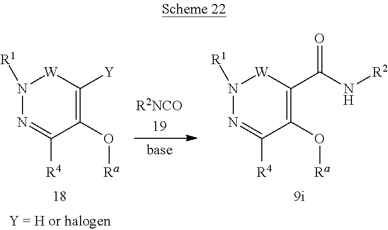

As shown in Scheme 23, compounds of Formula 18a (i.e. Formula 18 wherein W is C═S) can be prepared by reaction of compounds of Formula 18b (i.e. Formula 18 wherein W is C═O) with a thionation reagent such as Lawesson's reagent, tetraphosphorus decasulfide or diphosphorus pentasulfide in a solvent such as tetrahydrofuran or toluene. Typically, the reaction is carried out at temperatures ranging from 0 to 115° C. For a related example, see: J. Med. Chem. 2006, 49, 7826-7835.

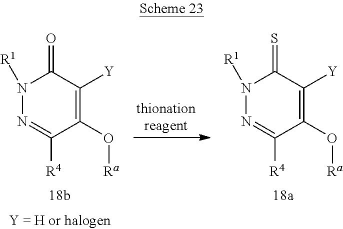

Compounds of Formula 18b are known in the literature. For examples, see: WO 2011/006910, J. Heterocyclic Chem. 1996, 33, 1579-1582, J. Heterocyclic Chem. 2000, 37, 1603-1606, US 2013/0331382, Tetrahedron Lett. 1980, 21 2939-2942 or WO 2010/009183, US 2010/0197651, J. Med. Chem. 2013, 56, 9837-9848, Synth. Commun. 2002, 32, 1675-1680.

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. For example, intermediates for the preparation of compounds of Formula 1 may contain aromatic nitro groups, which can be reduced to amino groups, and then be converted via reactions well known in the art such as the Sandmeyer reaction, to various halides, providing compounds of Formula 1. The above reactions can also in many cases be performed in alternate order.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane in CDCl₃ solution unless indicated otherwise; “s” means singlet, “d” means doublet, “t” means triplet, “q” means quartet, “m” means multiplet and “br s” means broad singlet. ¹⁹F NMR spectra are reported in ppm downfield from CFCl₃ in CDCl₃ unless indicated otherwise.

### Example 1

### Preparation of 2,3-Dihydro-5-hydroxy-2-methyl-3-oxo-N-[4-(trifluoromethyl)phenyl]-4-pyridazinecarboxamide

### Step A: Preparation of 5-methoxy-2-methyl-3-oxo-pyridazine-4-carboxylic acid

To a stirred solution of 5-methoxy-2-methyl-pyridazin-3-one (WO 2011/006910) (3.84 g, 27.4 mmol, 1.0 eq.) in tetrahydrofuran (117 mL) at 20° C. was added 2,2,6,6-tetramethylpiperidinyl-magnesium chloride lithium chloride complex (1 M in tetrahydrofuran/toluene, 41.1 mL, 41.1 mmol, 1.5 eq.). After stirring for 1 min., CO₂ was bubbled through the solution. The reaction was stirred for an additional 10 min. at 20° C., then allowed to warm to room temperature and 1 N HCl was added until the mixture was at pH 4. The aqueous solution was washed with ethyl acetate, then further acidified to pH 1. The acidic aqueous solution was extracted with ethyl acetate two times, the combined organics were dried over MgSO₄, filtered and concentrated in vacuo to provide 2.03 g (40% yield) of the title compound as a peach solid, which was 90% pure, and used as is in subsequent reactions.

¹H NMR (CDCl₃): δ 8.08 (s, 1H), 4.19 (s, 3H), 3.88 (s, 3H).

### Step B: Preparation of 5-methoxy-2-methyl-3-oxo-N-[4-(trifluoromethyl)phenyl]pyridazine-4-carboxamide

To a stirred solution of 5-methoxy-2-methyl-3-oxo-pyridazine-4-carboxylic acid (i.e. the product of Step A) (0.33 g, 1.8 mmol, 1.0 eq.) in tetrahydrofuran (4 mL) was added 4-trifluoromethyl-aniline (0.27 mL, 2.2 mmol, 1.2 eq.), triethylamine (0.75 mL, 5.4 mmol, 3.0 eq.) and propylphosphonic anydride (50% in ethyl acetate, 1.81 mL, 1.7 eq.). After stirring for 18 h, the reaction mixture was concentrated in vacuo to give the crude product, which was charged onto silica and purified by medium pressure liquid chromatography (0% to 100% ethyl acetate in hexanes as eluent) to yield 0.25 g (45% yield) of the title compound as a light yellow solid.

¹H NMR (CDCl₃): δ 11.79 (br s, 1H), 8.00 (s, 1H), 7.83 (d, 2H), 7.59 (d, 2H), 4.17 (s, 3H), 3.87 (s, 3H).

### Step C: Preparation of 2,3-Dihydro-5-hydroxy-2-methyl-3-oxo-N-[4-(trifluoromethyl)phenyl]-4-pyridazinecarboxamide

To a stirred solution of 5-methoxy-2-methyl-3-oxo-N-[4-(trifluoromethyl)phenyl]-pyridazine-4-carboxamide (i.e. the product of Step B) (0.29 g, 0.88 mmol, 1.0 eq.) in tetrahydrofuran (4.4 mL) was added NaOH (1 N in H₂O, 4.4 mL). After 18 h, the mixture was quenched with 1 N HCl to pH 1. The acidic solution was extracted with ethyl acetate, dried over MgSO₄, filtered and concentrated in vacuo. The crude material was charged onto silica and purified by medium pressure liquid chromatography (0% to 100% ethyl acetate in hexanes as eluent) to yield 0.18 g (66% yield) of the title compound as a white solid.

¹H NMR (CDCl₃): δ 14.56 (br s, 1H), 12.21 (br s, 1H), 7.80 (m, 3H), 7.64 (d, 2H), 3.81 (s, 3H).

### Example 2

### Preparation of 2,3-Dihydro-5-hydroxy-2-methyl-3-oxo-N-[4-(trifluoromethoxy)phenyl]-4-pyridazinecarboxamide

### Step A: Preparation of 5-methoxy-2-methyl-3-oxo-N-[4-(trifluoromethoxy)-phenyl]pyridazine-4-carboxamide

To a stirred solution of 5-methoxy-2-methyl-3-oxo-pyridazine-4-carboxylic acid (i.e. the product of Step A, EXAMPLE 1) (2.0 g, 10.9 mmol, 1.0 eq.) in dichloromethane (30 mL) was added oxalyl chloride (2.0 mL, 22.9 mmol, 2.1 eq.) slowly, followed by dimethylformamide (2 drops). After 18 h, the reaction mixture was concentrated in vacuo. Dichloromethane (30 mL) was added and the mixture was concentrated in vacuo again. The mixture was then redissolved in dichloromethane (30 mL) and 4-trifluoromethoxyaniline (2.95 mL, 21.8 mmol, 2.0 eq.) and triethylamine (3.2 mL, 22.9 mmol, 2.1 eq.) were added. After 24 h, the mixture was concentrated in vacuo, charged onto silica and purified by medium pressure liquid chromatography (0% to 100% ethyl acetate in hexanes as eluent) to yield 1.95 g (52% yield) of the title compound as a tan solid.

¹H NMR (CDCl₃): δ 11.58 (br s, 1H), 7.98 (s, 1H), 7.74 (m, 2H), 7.18 (m, 2H), 4.16 (s, 3H), 3.86 (s, 3H).

### Step B: Preparation of 2,3-Dihydro-5-hydroxy-2-methyl-3-oxo-N-[4-(trifluoromethoxy)phenyl]-4-pyridazinecarboxamide

To a stirred solution of 5-methoxy-2-methyl-3-oxo-N-[4-(trifluoromethoxy)phenyl]-pyridazine-4-carboxamide (i.e. the product of Step A, EXAMPLE 2) (0.2 g, 0.58 mmol, 1.0 eq.) in dichloromethane (4 mL) at 0° C. was added boron tribromide (1 M in dichloromethane, 1.2 mL, 1.2 mmol, 2.0 eq.). The reaction mixture was stirred at room temperature for 18 h, then quenched with 1 N NaOH. After stirring for 30 min., the reaction mixture was brought to pH 1 with 1 N HCl and extracted with dichloromethane. The organics were dried over MgSO₄, concentrated in vacuo, charged onto silica and purified by medium pressure liquid chromatography (0% to 100% ethyl acetate in hexanes as eluent) to yield 0.92 g (48% yield) of the title compound as a white solid.

¹H NMR (CDCl₃): δ 14.69 (s, 1H), 12.06 (br s, 1H), 7.78 (s, 1H), 7.70 (m, 2H), 7.23 (m, 2H), 3.80 (s, 3H).

### Example 3

### Preparation of 5-Chloro-2,3-dihydro-2-methyl-3-oxo-N-[4-(trifluoromethoxy)phenyl]-4-pyridazinecarboxamide

To a stirred solution of 2,3-Dihydro-5-hydroxy-2-methyl-3-oxo-N-[4-(trifluoromethoxy)phenyl]-4-pyridazinecarboxamide (i.e. the product of Step B, EXAMPLE 2) (0.15 g, 0.46 mmol, 1.0 eq.) in dichloromethane (2 mL) was added oxalyl chloride (0.085 mL, 0.97 mmol, 2.1 eq.) followed by dimethylformamide (2 drops). After stirring for 18 h, the mixture was concentrated in vacuo, charged onto silica and purified by medium pressure liquid chromatography (0% to 100% ethyl acetate in hexanes as eluent) to yield 0.051 g (32% yield) of the title compound as a yellow solid.

¹H NMR (CDCl₃): δ 11.35 (br s, 1H), 7.93 (s, 1H), 7.72 (m, 2H), 7.20 (m, 2H), 3.85 (s, 3H).

### Example 4

### Preparation of 5-Bromo-2,3-dihydro-2-methyl-3-oxo-N-[4-(trifluoromethoxy)phenyl]-4-pyridazinecarboxamide

To a stirred solution of 2,3-Dihydro-5-hydroxy-2-methyl-3-oxo-N-[4-(trifluoromethoxy)phenyl]-4-pyridazinecarboxamide (i.e. the product of Step B, EXAMPLE 2) (0.15 g, 0.46 mmol, 1.0 eq.) in dichloromethane (2 mL) was added oxalyl bromide (0.091 mL, 0.97 mmol, 2.1 eq.) followed by dimethylformamide (2 drops). After stirring for 18 h, the mixture was concentrated in vacuo, charged onto silica and purified by medium pressure liquid chromatography (0% to 100% ethyl acetate in hexanes as eluent) to yield 0.038 g (21% yield) of the title compound as a yellow solid.

¹H NMR (CDCl₃): δ 11.35 (br s, 1H), 8.10 (s, 1H), 7.73 (m, 2H), 7.21 (m, 2H), 3.86 (s, 3H).

### Example 5

### Preparation of 2,3-Dihydro-2-methyl-5-[(methylsulfonyl)oxy]-3-oxo-N-[4-trifluoromethoxy)phenyl]-4-pyridazinecarboxamide

To a stirred solution of 2,3-Dihydro-5-hydroxy-2-methyl-3-oxo-N-[4-(trifluoromethoxy)phenyl]-4-pyridazinecarboxamide (i.e. the product of Step B, EXAMPLE 2) (0.22 g, 0.67 mmol, 1.0 eq.) in dichloromethane (3 mL) was added methanesulfonyl chloride (0.077 mL, 1.0 mmol, 1.5 eq.) and triethylamine (0.19 mL, 1.34 mmol, 2 eq.). After stirring 18 h, the mixture was concentrated in vacuo, charged onto silica and purified by medium pressure liquid chromatography (0% to 100% ethyl acetate in hexanes as eluent) to yield 0.22 g (81% yield) of the title compound as a yellow solid.

¹H NMR (CDCl₃): δ 11.54 (br s, 1H), 8.08 (s, 1H), 7.71 (m, 2H), 7.22 (m, 2H), 3.92 (s, 3H), 3.55 (s, 3H).

### Example 6

### Preparation of 6-Methyl-3-[4-(trifluoromethoxy)phenyl]-2H-pyridazino[4,5-e]-1,3-oxazine-2,4,5(3H,6H)-trione

To a stirred solution of 2,3-Dihydro-5-hydroxy-2-methyl-3-oxo-N-[4-(trifluoromethoxy)phenyl]-4-pyridazinecarboxamide (i.e. the product of Step B, Example 2) (0.20 g, 0.61 mmol, 1 eq.) in tetrahydrofuran (4 mL) was added triethylamine (0.26 mL, 1.83 mmol, 3 eq.) followed by phosgene (15% in toluene, 1.31 mL, 1.83 mmol, 3 eq.). After 2.5 h, the reaction mixture was quenched with saturated aqueous sodium bicarbonate and extracted with ethyl acetate two times. The combined organics were dried over MgSO₄, filtered and concentrated in vacuo to give the crude product, which was charged onto silica and purified by medium pressure liquid chromatography (0% to 100% ethyl acetate in hexanes as eluent) to yield 0.083 g (38% yield) of the title compound as an off white solid.

¹H NMR (CDCl₃): δ 7.97 (s, 1H), 7.37 (m, 2H), 7.31 (m, 2H), 3.88 (s, 3H).

By the procedures described herein together with methods known in the art, the compounds disclosed in the Tables that follow can be prepared. The following abbreviations are used in the Tables which follow: i means iso, c means cyclo, n means normal, s means secondary, t means tertiary, Ac means acetate, Me means methyl, Et means ethyl, Bu means butyl, Pr means propyl, MeO means methoxy, CN means cyano, Ph means phenyl, and Bn means benzyl.

TABLE 1A

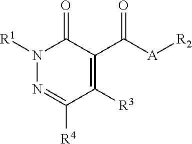

| R¹ is Me, A = NH, | R¹ is Me, A = NH, | R¹ is Me, A = NH, |
|---|---|---|
| R³ is OH and | R³ is OH and | R³ is OH and |
| R⁴ is H. | R⁴ is H. | R⁴ is H. |
| R² | R² | R² |
| 4-Cl—Ph | 2,4,5-tri-Br—Ph | 4-Cl-2-Br—Ph |
| 3-Cl—Ph | 3,4,5-tri-Br—Ph | 4-Cl-3-CF₃—Ph |
| 2-Cl—Ph | 2,3,4-tri-Me—Ph | 4-Cl-2-CF₃—Ph |
| 4-Br—Ph | 2,3,5-tri-Me—Ph | 4-Cl-3-OCF₃—Ph |
| 3-Br—Ph | 2,3,6-tri-Me—Ph | 4-Cl-2-OCF₃—Ph |
| 2-Br—Ph | 2,4,6-tri-Me—Ph | 2-Br-3-Cl—Ph |
| 4-F—Ph | 2,4,5-tri-Me—Ph | 2-Br-4-Cl—Ph |
| 3-F—Ph | 3,4,5-tri-Me—Ph | 2-Br-5-Cl—Ph |
| 2-F—Ph | 2,3,4-tri-OMe—Ph | 2-Br-6-Cl—Ph |
| 4-I—Ph | 2,3,5-tri-OMe—Ph | 2-Br-3-F—Ph |
| 3-I—Ph | 2,3,6-tri-OMe—Ph | 2-Br-4-F—Ph |
| 2-I—Ph | 2,4,6-tri-OMe—Ph | 2-Br-5-F—Ph |
| 4-Me—Ph | 2,4,5-tri-OMe—Ph | 2-Br-6-F—Ph |
| 3-Me—Ph | 3,4,5-tri-OMe—Ph | 2-Br-3-CF₃—Ph |
| 2-Me—Ph | 2,3,4-tri-CF₃—Ph | 2-Br-4-CF₃—Ph |
| 4-Et—Ph | 2,3,5-tri-CF₃—Ph | 2-Br-5-CF₃—Ph |
| 3-Et—Ph | 2,3,6-tri-CF₃—Ph | 2-Br-6-CF₃—Ph |
| 2-Et—Ph | 2,4,6-tri-CF₃—Ph | 2-Br-3-OCF₃—Ph |
| 4-Pr—Ph | 2,4,5-tri-CF₃—Ph | 2-Br-4-OCF₃—Ph |
| 3-Pr—Ph | 3,4,5-tri-CF₃—Ph | 2-Br-5-OCF₃—Ph |
| 2-Pr—Ph | 2,3,4-tri-OCF₃—Ph | 2-Br-6-OCF₃—Ph |
| 4-i-Pr—Ph | 2,3,5-tri-OCF₃—Ph | 3-Br-2-Cl—Ph |
| 3-i-Pr—Ph | 2,3,6-tri-OCF₃—Ph | 3-Br-4-Cl—Ph |
| 2-i-Pr—Ph | 2,4,6-tri-OCF₃—Ph | 3-Br-5-Cl—Ph |
| 4-c-Pr—Ph | 2,4,5-tri-OCF₃—Ph | 3-Br-6-Cl—Ph |
| 3-c-Pr—Ph | 3,4,5-tri-OCF₃—Ph | 3-Br-2-F—Ph |
| 2-c-Pr—Ph | 2,6-diF-4-CF₃—Ph | 3-Br-4-F—Ph |
| 4-CF₃—Ph | 2,6-diCl-4-CF₃—Ph | 3-Br-5-F—Ph |
| 3-CF₃—Ph | 2,6-diBr-4-CF₃—Ph | 3Br-6-F—Ph |
| 2-CF₃—Ph | 2,6-diF-4-OCF₃—Ph | 3-Br-2-CF₃—Ph |
| 4-MeO—Ph | 2,6-diCl-4-OCF₃—Ph | 3-Br-4-CF₃—Ph |
| 3-MeO—Ph | 2,6-diBr-4-OCF₃—Ph | 3-Br-5-CF₃—Ph |
| 2-MeO—Ph | 4-CHF₂O—Ph | 3-Br-6-CF₃—Ph |
| 4-EtO—Ph | 3-CHF₂O—Ph | 3-Br-2-OCF₃—Ph |
| 3-EtO—Ph | 2-CHF₂O—Ph | 3-Br-4-OCF₃—Ph |
| 2-EtO—Ph | 4-SF₅—Ph | 3-Br-5-OCF₃—Ph |
| 4-i-PrO—Ph | 3-SF₅—Ph | 3-Br-6-OCF₃—Ph |
| 3-i-PrO—Ph | 2-SF₅—Ph | 4-Br-3-Cl—Ph |
| 2-i-PrO—Ph | 4-SMe—Ph | 4-Br-2-Cl—Ph |
| 4-CF₃O—Ph | 3-SMe—Ph | 4-Br-3-F—Ph |
| 3-CF₃O—Ph | 2-SMe—Ph | 4-Br-2-F—Ph |
| 2-CF₃O—Ph | 4-SCF₃—Ph | 4-Br-3-CF₃—Ph |
| 4-CF₃CF₂O—Ph | 3-SCF₃—Ph | 4-Br-2-CF₃—Ph |
| 3-CF₃CF₂O—Ph | 2-SCF₃—Ph | 4-Br-3-OCF₃—Ph |
| 2-CF₃CF₂O—Ph | 4-SCN—Ph | 4-Br-2-OCF₃—Ph |
| 4-CN—Ph | 3-SCN—Ph | 2-pyridinyl |
| 3-CN—Ph | 2-SCN—Ph | 3-pyridinyl |
| 2-CN—Ph | 4-MeS(═O)—Ph | 4-pyridinyl |
| 4-NO₂—Ph | 3-MeS(═O)—Ph | 3-Me-2-pyridinyl |
| 3-NO₂—Ph | 2-MeS(═O)—Ph | 4-Me-2-pyridinyl |
| 2-NO₂—Ph | 4-CF₃S(═O)—Ph | 5-Me-2-pyridinyl |
| 4-Ac—Ph | 3-CF₃S(═O)—Ph | 6-Me-2-pyridinyl |
| 3-Ac—Ph | 2-CF₃S(═O)—Ph | 2-Me-3-pyridinyl |
| 2-Ac—Ph | 4-MeS(═O)₂—Ph | 4-Me-3-pyridinyl |
| 2-CF₃C(═O)—Ph | 3-MeS(═O)₂—Ph | 5-Me-3-pyridinyl |
| 3-CF₃C(═O)—Ph | 2-MeS(═O)₂—Ph | 6-Me-3-pyridinyl |
| 4-CF₃C(═O)—Ph | 4-CF₃S(═O)₂—Ph | 2-Me-4-pyridinyl |
| 4-MeC(═O)O—Ph | 3-CF₃S(═O)₂—Ph | 3-Me-4-pyridinyl |
| 3-MeC(═O)O—Ph | 2-CF₃S(═O)₂—Ph | 3-Cl-2-pyridinyl |
| 2-MeC(═O)O—Ph | 2-F-3-Cl—Ph | 4-Cl-2-pyridinyl |
| 4-MeC(═NH)—Ph | 2-F-4-Cl—Ph | 5-Cl-2-pyridinyl |
| 3-MeC(═NH)—Ph | 2-F-5-Cl—Ph | 6-Cl-2-pyridinyl |
| 2-MeC(═NH)—Ph | 2-F-6-Cl—Ph | 2-Cl-3-pyridinyl |
| 4-MeC(═NMe)—Ph | 2-F-3-Br—Ph | 4-Cl-3-pyridinyl |
| 3-MeC(═NMe)—Ph | 2-F-4-Br—Ph | 5-Cl-3-pyridinyl |
| 2-MeC(═NMe)—Ph | 2-F-5-Br—Ph | 6-Cl-3-pyridinyl |
| 4-MeC═(NOMe)—Ph | 2-F-6-Br—Ph | 2-Cl-4-pyridinyl |
| 3-MeC═(NOMe)—Ph | 2-F-3-CF₃—Ph | 3-Cl-4-pyridinyl |
| 2-MeC═(NOMe)—Ph | 2-F-4-CF₃—Ph | 3-F-2-pyridinyl |
| 4-MeC(═NOEt)—Ph | 2-F-5-CF₃—Ph | 4-F-2-pyridinyl |
| 3-MeC(═NOEt)—Ph | 2-F-6-CF₃—Ph | 5-F-2-pyridinyl |
| 2-MeC(═NOEt)—Ph | 2-F-3-OCF₃—Ph | 6-F-2-pyridinyl |
| 4-MeNHC(═O)—Ph | 2-F-4-OCF₃—Ph | 2-F-3-pyridinyl |
| 3-MeNHC(═O)—Ph | 2-F-5-OCF₃—Ph | 4-F-3-pyridinyl |
| 2-MeNHC(═O)—Ph | 2-F-6-OCF₃—Ph | 5-F-3-pyridinyl |
| 2,6-di-Cl—-Ph | 3-F-2-Cl—Ph | 6-F-3-pyridinyl |
| 2,5-di-Cl—Ph | 3-F-4-Cl—Ph | 2-F-4-pyridinyl |
| 2,4-di-Cl—Ph | 3-F-5-Cl—Ph | 3-F-4-pyridinyl |
| 3,3-di-Cl—Ph | 3-F-6-Cl—Ph | 3-Br-2-pyridinyl |
| 3,4-di-Cl—Ph | 3-F-2-Br—Ph | 4-Br-2-pyridinyl |
| 2,6-di-F—Ph | 3-F-4-Br—Ph | 5-Br-2-pyridinyl |
| 2,5-di-F—Ph | 3-F-5-Br—Ph | 6-Br-2-pyridinyl |
| 2,4-di-F—Ph | 3-F-6-Br—Ph | 2-Br-3-pyridinyl |
| 3,3-di-F—Ph | 3-F-2-CF₃—Ph | 4-Br-3-pyridinyl |
| 3,4-di-F—Ph | 3-F-4-CF₃—Ph | 5-Br-3-pyridinyl |
| 2,6-di-Br—Ph | 3-F-5-CF₃—Ph | 6-Br-3-pyridinyl |
| 2,5-di-Br—Ph | 3-F-6-CF₃—Ph | 2-Br-4-pyridinyl |
| 2,4-di-Br—Ph | 3-F-2-OCF₃—Ph | 3-Br-4-pyridinyl |
| 3,3-di-Br—Ph | 3-F-4-OCF₃—Ph | 3-OCF₃-2-pyridinyl |
| 3,4-di-Br—Ph | 3-F-5-OCF₃—Ph | 4-OCF₃-2-pyridinyl |
| 2,6-di-Me—Ph | 3-F-6-OCF₃—Ph | 5-OCF₃-2-pyridinyl |
| 2,5-di-Me—Ph | 4-F-3-Cl—Ph | 6-OCF₃-2-pyridinyl |
| 2,4-di-Me—Ph | 4-F-2-Cl—Ph | 2-OCF₃-3-pyridinyl |
| 3,3-di-Me—Ph | 4-F-3-Br—Ph | 4-OCF₃-3-pyridinyl |
| 3,4-di-Me—Ph | 4-F-2-Br—Ph | 5-OCF₃-3-pyridinyl |
| 2,6-di-Et—Ph | 4-F-3-CF₃—Ph | 6-OCF₃-3-pyridinyl |
| 2,5-di-Et—Ph | 4-F-2-CF₃—Ph | 2-OCF₃-4-pyridinyl |
| 2,4-di-Et—Ph | 4-F-3-OCF₃—Ph | 3-OCF₃-4-pyridinyl |
| 3,3-di-Et—Ph | 4-F-2-OCF₃—Ph | 3-CF₃-2-pyridinyl |
| 3,4-di-Et—Ph | 2-Cl-3-F—Ph | 4-CF₃-2-pyridinyl |
| 2,6-di-CF₃—Ph | 2-Cl-4-F—Ph | 5-CF₃-2-pyridinyl |
| 2,5-di-CF₃—Ph | 2-Cl-5-F—Ph | 6-CF₃-2-pyridinyl |
| 2,4-di-CF₃—Ph | 2-Cl-6-F—Ph | 2-CF₃-3-pyridinyl |
| 3,3-di-CF₃—Ph | 2-Cl-3-Br—Ph | 4-CF₃-3-pyridinyl |
| 3,4-di-CF₃—Ph | 2-Cl-4-Br—Ph | 5-CF₃-3-pyridinyl |
| 4-CHF₂—Ph | 2-Cl-5-Br—Ph | 6-CF₃-3-pyridinyl |
| 3-CHF₂—Ph | 2-Cl-6-Br—Ph | 2-CF₃-4-pyridinyl |
| 2-CHF₂—Ph | 2-Cl-3-CF₃—Ph | 3-CF₃-4-pyridinyl |
| 2,6-di-MeO—Ph | 2-Cl-4-CF₃—Ph | 2-thienyl |
| 2,5-di-MeO—Ph | 2-Cl-5-CF₃—Ph | 3-thienyl |
| 2,4-di-MeO—Ph | 2-Cl-6-CF₃—Ph | benzyl |
| 3,3-di-MeO—Ph | 2-Cl-3-OCF₃—Ph | 4-Cl-benzyl |
| 3,4-di-MeO—Ph | 2-Cl-4-OCF₃—Ph | 3-Cl-benzyl |
| 2,6-di-CF₃O—Ph | 2-Cl-5-OCF₃—Ph | 2-Cl-benzyl |
| 2,5-di-CF₃O—Ph | 2-Cl-6-OCF₃—Ph | 4-Br-benzyl |
| 2,4-di-CF₃O—Ph | 3-Cl-2-F—Ph | 3-Br-benzyl |
| 3,3-di-CF₃O—Ph | 3-Cl-4-F—Ph | 2-Br-benzyl |
| 3,4-di-CF₃O—Ph | 3-Cl-5-F—Ph | 4-F-benzyl |
| 2,3,4-tri-F—Ph | 3-Cl-6-F—Ph | 3-F-benzyl |
| 2,3,5-tri-F—Ph | 3-Cl-2-Br—Ph | 2-F-benzyl |
| 2,3,6-tri-F—Ph | 3-Cl-4-Br—Ph | 4-Me-benzyl |
| 2,4,6-tri-F—Ph | 3-Cl-5-Br—Ph | 3-Me-benzyl |
| 2,4,5-tri-F—Ph | 3-Cl-6-Br—Ph | 2-Me-benzyl |
| 3,4,5-tri-F—Ph | 3-Cl-2-CF₃—Ph | 4-CF₃-benzyl |
| 2,3,4-tri-Cl—Ph | 3-Cl-4-CF₃—Ph | 3-CF₃-benzyl |
| 2,3,5-tri-Cl—Ph | 3-Cl-5-CF₃—Ph | 2-CF₃-benzyl |
| 2,3,6-tri-Cl—Ph | 3-Cl-6-CF₃—Ph | 4-MeO-benzyl |
| 2,4,6-tri-Cl—Ph | 3-Cl-2-OCF₃—Ph | 3-MeO-benzyl |
| 2,4,5-tri-Cl—Ph | 3-Cl-4-OCF₃—Ph | 2-MeO-benzyl |
| 3,4,5-tri-Cl—Ph | 3-Cl-5-OCF₃—Ph | 4-CN-benzyl |
| 2,3,4-tri-Br—Ph | 3-Cl-6-OCF₃—Ph | 3-CN-benzyl |
| 2,3,5-tri-Br—Ph | 4-Cl-3-F—Ph | 2-CN-benzyl |
| 2,3,6-tri-Br—Ph | 4-Cl-2-F—Ph | 1-naphthalenyl |
| 2,4,6-tri-Br—Ph | 4-Cl-3-Br—Ph | 2-naphthalenyl |

The present disclosure also includes Tables 1B through 738B, each of which is constructed the same as Table 1A above, except that the row heading in Table 1A (i.e. “R¹ is Me, A=NH, R³ is OH and R⁴ is H”) is replaced with the respective row heading shown below. For Example, in Table 1B the row heading is “R¹ is Me, A=NH, R³ is Cl and R⁴ is H” and R² is as defined in Table 1A above.

| Table | Row Heading |
|---|---|
| 1B | R¹ is Me, A = NH, R³ is Cl and R⁴ is H. |
| 2B | R¹ is Me, A = NH, R³ is Br and R⁴ is H. |
| 3B | R¹ is Me, A = NH, R³ is OS(═O)₂Me |
| | and R⁴ is H. |
| 4B | R¹ is Me, A = NH, R³ is OS(═O)₂Et and |
| | R⁴ is H. |
| 5B | R¹ is Me, A = NH, R³ is OS(═O)₂Pr and |
| | R⁴ is H. |
| 6B | R¹ is Me, A = NH, R³ is OS(═O)₂i-Pr |
| | and R⁴ is H. |
| 7B | R¹ is Me, A = NH, R³ is OS(═O)₂Bu |
| | and R⁴ is H. |
| 8B | R¹ is Me, A = NH, R³ is OS(═O)₂t-Bu |
| | and R⁴ is H. |
| 9B | R¹ is Me, A = NH, R³ is OS(═O)₂c-Pr |
| | and R⁴ is H. |
| 10B | R¹ is Me, A = NH, R³ is OS(═O)₂OMe |
| | and R⁴ is H. |
| 11B | R¹ is Me, A = NH, R³ is OS(═O)₂Ph |
| | and R⁴ is H. |
| 12B | R¹ is Me, A = NH, R³ is OS(═O)₂Bn |
| | and R⁴ is H. |
| 13B | R¹ is Me, A = NH, R³ is OC(═O)Me and |
| | R⁴ is H. |
| 14B | R¹ is Me, A = NH, R³ is OC(═O)Et and |
| | R⁴ is H. |
| 15B | R¹ is Me, A = NH, R³ is OC(═O)Pr and |
| | R⁴ is H. |
| 16B | R¹ is Me, A = NH, R³ is OC(═O)i-Pr |
| | and R⁴ is H. |
| 17B | R¹ is Me, A = NH, R³ is OC(═O)Bu and |
| | R⁴ is H. |
| 18B | R¹ is Me, A = NH, R³ is OC(═O)t-Bu |
| | and R⁴ is H. |
| 19B | R¹ is Me, A = NH, R³ is OC(═O)c-Pr |
| | and R⁴ is H. |
| 20B | R¹ is Me, A = NH, R³ is OC(═O)Ph and |
| | R⁴ is H. |
| 21B | R¹ is Me, A = NH, R³ is OC(═O)Bn and |
| | R⁴ is H. |
| 22B | R¹ is Me, A = NH, R³ is OC(═O)OMe |
| | and R⁴ is H. |
| 23B | R¹ is Me, A = NH, R³ is OC(═O)OEt |
| | and R⁴ is H. |
| 24B | R¹ is Me, A = NH, R³ is OC(═O)OPr |
| | and R⁴ is H. |
| 25B | R¹ is Me, A = NH, R³ is OC(═O)Oi-Pr |
| | and R⁴ is H. |
| 26B | R¹ is Me, A = NH, R³ is OC(═O)OBu |
| | and R⁴ is H. |
| 27B | R¹ is Me, A = NH, R³ is OC(═O)Ot-Bu |
| | and R⁴ is H. |
| 28B | R¹ is Me, A = NH, R³ is OC(═O)Oc-Pr |
| | and R⁴ is H. |
| 29B | R¹ is Me, A = NH, R³ is OC(═O)OPh |
| | and R⁴ is H. |
| 30B | R¹ is Me, A = NH, R³ is OC(═O)OBn |
| | and R⁴ is H. |
| 31B | R¹ is Me, A and R³ are taken together to |
| | form NC(═O)O and R⁴ is H. |
| 32B | R¹ is Me, A and R³ are taken together to |
| | form NCH₂O and R⁴ is H. |
| 33B | R¹ is Me, A and R³ are taken together to |
| | form NS(═O)₂O and R⁴ is H. |
| 34B | R¹ is Me, A = NH, R³ is SMe and R⁴ is |
| | H. |
| 35B | R¹ is Me, A = NH, R³ is S(═O)Me and |
| | R⁴ is H. |
| 36B | R¹ is Me, A = NH, R³ is S(═O)₂Me and |
| | R⁴ is H. |
| 37B | R¹ is Me, A = NC(═O)Me, R³ is |
| | OC(═O)Me and R⁴ is H. |
| 38B | R¹ is Me, A = NC(═O)Et, R³ is |
| | OC(═O)Et and R⁴ is H. |
| 39B | R¹ is Me, A = NC(═O)i-Pr, R³ is |
| | OC(═O)i-Pr and R⁴ is H. |
| 40B | R¹ is Me, A = NC(═O)Ph, R³ is |
| | OC(═O)Ph and R⁴ is H. |
| 41B | R¹ is Me, A = NC(═O)OMe, R³ is |
| | OC(═O)OMe and R⁴ is H. |
| 42B | R¹ is Me, A = NH, R³ is Cl and R⁴ is Cl. |
| 43B | R¹ is Me, A = NH, R³ is Br and R⁴ is |
| | Cl. |
| 44B | R¹ is Me, A = NH, R³ is OS(═O)₂Me |
| | and R⁴ is Cl. |
| 45B | R¹ is Me, A = NH, R³ is OS(═O)₂Et and |
| | R⁴ is Cl. |
| 46B | R¹ is Me, A = NH, R³ is OS(═O)₂Pr and |
| | R⁴ is Cl. |
| 47B | R¹ is Me, A = NH, R³ is OS(═O)₂i-Pr |
| | and R⁴ is Cl. |
| 48B | R¹ is Me, A = NH, R³ is OS(═O)₂Bu |
| | and R⁴ is Cl. |
| 49B | R¹ is Me, A = NH, R³ is OS(═O)₂t-Bu |
| | and R⁴ is Cl. |
| 50B | R¹ is Me, A = NH, R³ is OS(═O)₂c-Pr |
| | and R⁴ is Cl. |
| 51B | R¹ is Me, A = NH, R³ is OS(═O)₂OMe |
| | and R⁴ is Cl. |
| 52B | R¹ is Me, A = NH, R³ is OS(═O)₂Ph |
| | and R⁴ is Cl. |
| 53B | R¹ is Me, A = NH, R³ is OS(═O)₂Bn |
| | and R⁴ is Cl. |
| 54B | R¹ is Me, A = NH, R³ is OC(═O)Me and |
| | R⁴ is Cl. |
| 55B | R¹ is Me, A = NH, R³ is OC(═O)Et and |
| | R⁴ is Cl. |
| 56B | R¹ is Me, A = NH, R³ is OC(═O)Pr and |
| | R⁴ is Cl. |
| 57B | R¹ is Me, A = NH, R³ is OC(═O)i-Pr |
| | and R⁴ is Cl. |
| 58B | R¹ is Me, A = NH, R³ is OC(═O)Bu and |
| | R⁴ is Cl. |
| 59B | R¹ is Me, A = NH, R³ is OC(═O)t-Bu |
| | and R⁴ is Cl. |
| 60B | R¹ is Me, A = NH, R³ is OC(═O)c-Pr |
| | and R⁴ is Cl. |
| 61B | R¹ is Me, A = NH, R³ is OC(═O)Ph and |
| | R⁴ is Cl. |
| 62B | R¹ is Me, A = NH, R³ is OC(═O)Bn and |
| | R⁴ is Cl. |
| 63B | R¹ is Me, A = NH, R³ is OC(═O)OMe |
| | and R⁴ is Cl. |
| 64B | R¹ is Me, A = NH, R³ is OC(═O)OEt |
| | and R⁴ is Cl. |
| 65B | R¹ is Me, A = NH, R³ is OC(═O)OPr |
| | and R⁴ is Cl. |
| 66B | R¹ is Me, A = NH, R³ is OC(═O)Oi-Pr |
| | and R⁴ is Cl. |
| 67B | R¹ is Me, A = NH, R³ is OC(═O)OBu |
| | and R⁴ is Cl. |
| 68B | R¹ is Me, A = NH, R³ is OC(═O)Ot-Bu |
| | and R⁴ is Cl. |
| 69B | R¹ is Me, A = NH, R³ is OC(═O)Oc-Pr |
| | and R⁴ is Cl. |
| 70B | R¹ is Me, A = NH, R³ is OC(═O)OPh |
| | and R⁴ is Cl. |
| 71B | R¹ is Me, A = NH, R³ is OC(═O)OBn |
| | and R⁴ is Cl. |
| 72B | R¹ is Me, A and R³ are taken together to |
| | form NC(═O)O and R⁴ is Cl. |
| 73B | R¹ is Me, A and R³ are taken together to |
| | form NCH₂O and R⁴ is Cl. |
| 74B | R¹ is Me, A and R³ are taken together to |
| | form NS(═O)₂O and R⁴ is Cl. |
| 75B | R¹ is Me, A = NH, R³ is SMe and R⁴ is |
| | Cl. |
| 76B | R¹ is Me, A = NH, R³ is S(═O)Me and |
| | R⁴ is Cl. |
| 77B | R¹ is Me, A = NH, R³ is S(═O)₂Me and |
| | R⁴ is Cl. |
| 78B | R¹ is Me, A = NC(═O)Me, R³ is |
| | OC(═O)Me and R⁴ is Cl. |
| 79B | R¹ is Me, A = NC(═O)Et, R³ is |
| | OC(═O)Et and R⁴ is Cl. |
| 80B | R¹ is Me, A = NC(═O)i-Pr, R³ is |
| | OC(═O)i-Pr and R⁴ is Cl. |
| 81B | R¹ is Me, A = NC(═O)Ph, R³ is |
| | OC(═O)Ph and R⁴ is Cl. |
| 82B | R¹ is Me, A = NC(═O)OMe, R³ is |
| | OC(═O)OMe and R⁴ is Cl. |
| 83B | R¹ is Me, A = NH, R³ is Cl and R⁴ is |
| | OMe. |
| 84B | R¹ is Me, A = NH, R³ is Br and R⁴ is |
| | OMe. |
| 85B | R¹ is Me, A = NH, R³ is OS(═O)₂Me |
| | and R⁴ is OMe. |
| 86B | R¹ is Me, A = NH, R³ is OS(═O)₂Et and |
| | R⁴ is OMe. |
| 87B | R¹ is Me, A = NH, R³ is OS(═O)₂Pr and |
| | R⁴ is OMe. |
| 88B | R¹ is Me, A = NH, R³ is OS(═O)₂i-Pr |
| | and R⁴ is OMe. |
| 89B | R¹ is Me, A = NH, R³ is OS(═O)₂Bu |
| | and R⁴ is OMe. |
| 90B | R¹ is Me, A = NH, R³ is OS(═O)₂t-Bu |
| | and R⁴ is OMe. |
| 91B | R¹ is Me, A = NH, R³ is OS(═O)₂c-Pr |
| | and R⁴ is OMe. |
| 92B | R¹ is Me, A = NH, R³ is OS(═O)₂OMe |
| | and R⁴ is OMe. |
| 93B | R¹ is Me, A = NH, R³ is OS(═O)₂Ph |
| | and R⁴ is OMe. |
| 94B | R¹ is Me, A = NH, R³ is OS(═O)₂Bn |
| | and R⁴ is OMe. |
| 95B | R¹ is Me, A = NH, R³ is OC(═O)Me and |
| | R⁴ is OMe. |
| 96B | R¹ is Me, A = NH, R³ is OC(═O)Et and |
| | R⁴ is OMe. |
| 97B | R¹ is Me, A = NH, R³ is OC(═O)Pr and |
| | R⁴ is OMe. |
| 98B | R¹ is Me, A = NH, R³ is OC(═O)i-Pr |
| | and R⁴ is OMe. |
| 99B | R¹ is Me, A = NH, R³ is OC(═O)Bu and |
| | R⁴ is OMe. |
| 100B | R¹ is Me, A = NH, R³ is OC(═O)t-Bu |
| | and R⁴ is OMe. |
| 101B | R¹ is Me, A = NH, R³ is OC(═O)c-Pr |
| | and R⁴ is OMe. |
| 102B | R¹ is Me, A = NH, R³ is OC(═O)Ph and |
| | R⁴ is OMe. |
| 103B | R¹ is Me, A = NH, R³ is OC(═O)Bn and |
| | R⁴ is OMe. |
| 104B | R¹ is Me, A = NH, R³ is OC(═O)OMe |
| | and R⁴ is OMe. |
| 105B | R¹ is Me, A = NH, R³ is OC(═O)OEt |
| | and R⁴ is OMe. |
| 106B | R¹ is Me, A = NH, R³ is OC(═O)OPr |
| | and R⁴ is OMe. |
| 107B | R¹ is Me, A = NH, R³ is OC(═O)Oi-Pr |
| | and R⁴ is OMe. |
| 108B | R¹ is Me, A = NH, R³ is OC(═O)OBu |
| | and R⁴ is OMe. |
| 109B | R¹ is Me, A = NH, R³ is OC(═O)Ot-Bu |
| | and R⁴ is OMe. |
| 110B | R¹ is Me, A = NH, R³ is 0C(═O)Oc-Pr |
| | and R⁴ is OMe. |
| 111B | R¹ is Me, A = NH, R³ is OC(═O)OPh |
| | and R⁴ is OMe. |
| 112B | R¹ is Me, A = NH, R³ is OC(═O)OBn |
| | and R⁴ is OMe. |
| 113B | R¹ is Me, A and R³ are taken together to |
| | form NC(═O)O and R⁴ is OMe. |
| 114B | R¹ is Me, A and R³ are taken together to |
| | form NCH₂O and R⁴ is OMe. |
| 115B | Ri is Me, A and R³ are taken together to |
| | form NS(═O)₂O and R⁴ is OMe. |
| 116B | R¹ is Me, A = NH, R³ is SMe and R⁴ is |
| | OMe. |
| 117B | R¹ is Me, A = NH, R³ is S(═O)Me and |
| | R⁴ is OMe. |
| 118B | R¹ is Me, A = NH, R³ is S(═O)₂Me and |
| | R⁴ is OMe. |
| 119B | R¹ is Me, A = NC(═O)Me, R³ is |
| | OC(═O)Me and R⁴ is OMe. |
| 120B | R¹ is Me, A = NC(═O)Et, R³ is |
| | OC(═O)Et and R⁴ is OMe. |
| 121B | R¹ is Me, A = NC(═O)i-Pr, R³ is |
| | OC(═O)i-Pr and R⁴ is OMe. |
| 122B | R¹ is Me, A = NC(═O)Ph, R³ is |
| | OC(═O)Ph and R⁴ is OMe. |
| 123B | R¹ is Me, A = NC(═O)OMe, R³ is |
| | OC(═O)OMe and R⁴ is OMe. |
| 124B | R¹ is Et, A = NH, R³ is Cl and R⁴ is H. |
| 125B | R¹ is Et, A = NH, R³ is Br and R⁴ is H. |
| 126B | R¹ is Et, A = NH, R³ is OS(═O)₂Me and |
| | R⁴ is H. |
| 127B | R¹ is Et, A = NH, R³ is OS(═O)₂Et and |
| | R⁴ is H. |
| 128B | R¹ is Et, A = NH, R³ is OS(═O)₂Pr and |
| | R⁴ is H. |
| 129B | R¹ is Et, A = NH, R³ is OS(═O)₂i-Pr |
| | and R⁴ is H. |
| 130B | R¹ is Et, A = NH, R³ is OS(═O)₂Bu and |
| | R⁴ is H. |
| 131B | R¹ is Et, A = NH, R³ is OS(═O)₂t-Bu |
| | and R⁴ is H. |
| 132B | R¹ is Et, A = NH, R³ is OS(═O)₂c-Pr |
| | and R⁴ is H. |
| 133B | R¹ is Et, A = NH, R³ is OS(═O)₂OMe |
| | and R⁴ is H. |
| 134B | R¹ is Et, A = NH, R³ is OS(═O)₂Ph and |
| | R⁴ is H. |
| 135B | R¹ is Et, A = NH, R³ is OS(═O)₂Bn and |
| | R⁴ is H. |
| 136B | R¹ is Et, A = NH, R³ is OC(═O)Me and |
| | R⁴ is H. |
| 137B | R¹ is Et, A = NH, R³ is OC(═O)Et and |
| | R⁴ is H. |
| 138B | R¹ is Et, A = NH, R³ is OC(═O)Pr and |
| | R⁴ is H. |
| 139B | R¹ is Et, A = NH, R³ is OC(═O)i-Pr and |
| | R⁴ is H. |
| 140B | R¹ is Et, A = NH, R³ is OC(═O)Bu and |
| | R⁴ is H. |
| 141B | R¹ is Et, A = NH, R³ is OC(═O)t-Bu and |
| | R⁴ is H. |
| 142B | R¹ is Et, A = NH, R³ is OC(═O)c-Pr and |
| | R⁴ is H. |
| 143B | R¹ is Et, A = NH, R³ is OC(═O)Ph and |
| | R⁴ is H. |
| 144B | R¹ is Et, A = NH, R³ is OC(═O)Bn and |
| | R⁴ is H. |
| 145B | R¹ is Et, A = NH, R³ is OC(═O)OMe |
| | and R⁴ is H. |
| 146B | R¹ is Et, A = NH, R³ is OC(═O)OEt and |
| | R⁴ is H. |
| 147B | R¹ is Et, A = NH, R³ is OC(═O)OPr and |
| | R⁴ is H. |
| 148B | R¹ is Et, A = NH, R³ is OC(═O)Oi-Pr |
| | and R⁴ is H. |
| 149B | R¹ is Et, A = NH, R³ is OC(═O)OBu |
| | and R⁴ is H. |
| 150B | R¹ is Et, A = NH, R³ is OC(═O)Ot-Bu |
| | and R⁴ is H. |
| 151B | R¹ is Et, A = NH, R³ is OC(═O)Oc-Pr |
| | and R⁴ is H. |
| 152B | R¹ is Et, A = NH, R³ is OC(═O)OPh and |
| | R⁴ is H. |
| 153B | R¹ is Et, A = NH, R³ is OC(═O)OBn |
| | and R⁴ is H. |
| 154B | R¹ is Et, A and R³ are taken together to |
| | form NC(═O)O and R⁴ is H. |
| 155B | R¹ is Et, A and R³ are taken together to |
| | form NCH₂O and R⁴ is H. |
| 156B | R¹ is Et, A and R³ are taken together to |
| | form NS(═O)₂O and R⁴ is H. |
| 157B | R¹ is Et, A = NH, R³ is SMe and R⁴ is |
| | H. |
| 158B | R¹ is Et, A = NH, R³ is S(═O)Me and |
| | R⁴ is H. |
| 159B | R¹ is Et, A = NH, R³ is S(═O)₂Me and |
| | R⁴ is H. |
| 160B | R¹ is Et, A = NC(═O)Me, R³ is |
| | OC(═O)Me and R⁴ is H. |
| 161B | R¹ is Et, A = NC(═O)Et, R³ is |
| | OC(═O)Et and R⁴ is H. |
| 162B | R¹ is Et, A = NC(═O)i-Pr, R³ is |
| | OC(═O)i-Pr and R⁴ is H. |
| 163B | R¹ is Et, A = NC(═O)Ph, R³ is |
| | OC(═O)Ph and R⁴ is H. |
| 164B | R¹ is Et, A = NC(═O)OMe, R³ is |
| | OC(═O)OMe and R⁴ is H. |
| 165B | R¹ is Et, A = NH, R³ is Cl and R⁴ is Cl. |
| 166B | R¹ is Et, A = NH, R³ is Br and R⁴ is Cl. |
| 167B | R¹ is Et, A = NH, R³ is OS(═O)₂Me and |
| | R⁴ is Cl. |
| 168B | R¹ is Et, A = NH, R³ is OS(═O)₂Et and |
| | R⁴ is Cl. |
| 169B | R¹ is Et, A = NH, R³ is OS(═O)₂Pr and |
| | R⁴ is Cl. |
| 170B | R¹ is Et, A = NH, R³ is OS(═O)₂i-Pr |
| | and R⁴ is Cl. |
| 171B | R¹ is Et, A = NH, R³ is OS(═O)₂Bu and |
| | R⁴ is Cl. |
| 172B | R¹ is Et, A = NH, R³ is OS(═O)₂t-Bu |
| | and R⁴ is Cl. |
| 173B | R¹ is Et, A = NH, R³ is OS(═O)₂c-Pr |
| | and R⁴ is Cl. |
| 174B | R¹ is Et, A = NH, R³ is OS(═O)₂OMe |
| | and R⁴ is Cl. |
| 175B | R¹ is Et, A = NH, R³ is OS(═O)₂Ph and |
| | R⁴ is Cl. |
| 176B | R¹ is Et, A = NH, R³ is OS(═O)₂Bn and |
| | R⁴ is Cl. |
| 177B | R¹ is Et, A = NH, R³ is OC(═O)Me and |
| | R⁴ is Cl. |
| 178B | R¹ is Et, A = NH, R³ is OC(═O)Et and |
| | R⁴ is Cl. |
| 179B | R¹ is Et, A = NH, R³ is OC(═O)Pr and |
| | R⁴ is Cl. |
| 180B | R¹ is Et, A = NH, R³ is OC(═O)i-Pr and |
| | R⁴ is Cl. |
| 181B | R¹ is Et, A = NH, R³ is OC(═O)Bu and |
| | R⁴ is Cl. |
| 182B | R¹ is Et, A = NH, R³ is OC(═O)t-Bu and |
| | R⁴ is Cl. |
| 183B | R¹ is Et, A = NH, R³ is OC(═O)c-Pr and |
| | R⁴ is Cl. |
| 184B | R¹ is Et, A = NH, R³ is OC(═O)Ph and |
| | R⁴ is Cl. |
| 185B | R¹ is Et, A = NH, R³ is OC(═O)Bn and |
| | R⁴ is Cl. |
| 186B | R¹ is Et, A = NH, R³ is OC(═O)OMe |
| | and R⁴ is Cl. |
| 187B | R¹ is Et, A = NH, R³ is OC(═O)OEt and |
| | R⁴ is Cl. |
| 188B | R¹ is Et, A = NH, R³ is OC(═O)OPr and |
| | R⁴ is Cl. |
| 189B | R¹ is Et, A = NH, R³ is OC(═O)Oi-Pr |
| | and R⁴ is Cl. |
| 190B | R¹ is Et, A = NH, R³ is OC(═O)OBu |
| | and R⁴ is Cl. |
| 191B | R¹ is Et, A = NH, R³ is OC(═O)Ot-Bu |
| | and R⁴ is Cl. |
| 192B | R¹ is Et, A = NH, R³ is OC(═O)Oc-Pr |
| | and R⁴ is Cl. |
| 193B | R¹ is Et, A = NH, R³ is OC(═O)OPh and |
| | R⁴ is Cl. |
| 194B | R¹ is Et, A = NH, R³ is OC(═O)OBn |
| | and R⁴ is Cl. |
| 195B | R¹ is Et, A and R³ are taken together to |
| | form NC(═O)O and R⁴ is Cl. |
| 196B | R¹ is Et, A and R³ are taken together to |
| | form NCH₂O and R⁴ is Cl. |
| 197B | R¹ is Et, A and R³ are taken together to |
| | form NS(═O)₂O and R⁴ is Cl. |
| 198B | R¹ is Et, A = NH, R³ is SMe and R⁴ is |
| | Cl. |
| 199B | R¹ is Et, A = NH, R³ is S(═O)Me and |
| | R⁴ is Cl. |
| 200B | R¹ is Et, A = NH, R³ is S(═O)₂Me and |
| | R⁴ is Cl. |
| 201B | R¹ is Et, A = NC(═O)Me, R³ is |
| | OC(═O)Me and R⁴ is Cl. |
| 202B | R¹ is Et, A = NC(═O)Et, R³ is |
| | OC(═O)Et and R⁴ is Cl. |
| 203B | R¹ is Et, A = NC(═O)i-Pr, R³ is |
| | OC(═O)i-Pr and R⁴ is Cl. |
| 204B | R¹ is Et, A = NC(═O)Ph, R³ is |
| | OC(═O)Ph and R⁴ is Cl. |
| 205B | R¹ is Et, A = NC(═O)OMe, R³ is |
| | OC(═O)OMe and R⁴ is Cl. |
| 206B | R¹ is Et, A = NH, R³ is Cl and R⁴ is |
| | OMe. |
| 207B | R¹ is Et, A = NH, R³ is Br and R⁴ is |
| | OMe. |
| 208B | R¹ is Et, A = NH, R³ is OS(═O)₂Me and |
| | R⁴ is OMe. |
| 209B | R¹ is Et, A = NH, R³ is OS(═O)₂Et and |
| | R⁴ is OMe. |
| 210B | R¹ is Et, A = NH, R³ is OS(═O)₂Pr and |
| | R⁴ is OMe. |
| 211B | R¹ is Et, A = NH, R³ is OS(═O)₂i-Pr |
| | and R⁴ is OMe. |
| 212B | R¹ is Et, A = NH, R³ is OS(═O)₂Bu and |
| | R⁴ is OMe. |
| 213B | R¹ is Et, A = NH, R³ is OS(═O)₂t-Bu |
| | and R⁴ is OMe. |
| 214B | R¹ is Et, A = NH, R³ is OS(═O)₂c-Pr |
| | and R⁴ is OMe. |
| 215B | R¹ is Et, A = NH, R³ is OS(═O)₂OMe |
| | and R⁴ is OMe. |
| 216B | R¹ is Et, A = NH, R³ is OS(═O)₂Ph and |
| | R⁴ is OMe. |
| 217B | R¹ is Et, A = NH, R³ is OS(═O)₂Bn and |
| | R⁴ is OMe. |
| 218B | R¹ is Et, A = NH, R³ is OC(═O)Me and |
| | R⁴ is OMe. |
| 219B | R¹ is Et, A = NH, R³ is OC(═O)Et and |
| | R⁴ is OMe. |
| 220B | R¹ is Et, A = NH, R³ is OC(═O)Pr and |
| | R⁴ is OMe. |
| 221B | R¹ is Et, A = NH, R³ is OC(═O)i-Pr and |
| | R⁴ is OMe. |
| 222B | R¹ is Et, A = NH, R³ is OC(═O)Bu and |
| | R⁴ is OMe. |
| 223B | R¹ is Et, A = NH, R³ is OC(═O)t-Bu and |
| | R⁴ is OMe. |
| 224B | R¹ is Et, A = NH, R³ is OC(═O)c-Pr and |
| | R⁴ is OMe. |
| 225B | R¹ is Et, A = NH, R³ is OC(═O)Ph and |
| | R⁴ is OMe. |
| 226B | R¹ is Et, A = NH, R³ is OC(═O)Bn and |
| | R⁴ is OMe. |
| 227B | R¹ is Et, A = NH, R³ is OC(═O)OMe |
| | and R⁴ is OMe. |
| 228B | R¹ is Et, A = NH, R³ is OC(═O)OEt and |
| | R⁴ is OMe. |
| 229B | R¹ is Et, A = NH, R³ is OC(═O)OPr and |
| | R⁴ is OMe. |
| 230B | R¹ is Et, A = NH, R³ is OC(═O)Oi-Pr |
| | and R⁴ is OMe. |
| 231B | R¹ is Et, A = NH, R³ is OC(═O)OBu |
| | and R⁴ is OMe. |
| 232B | R¹ is Et, A = NH, R³ is OC(═O)Ot-Bu |
| | and R⁴ is OMe. |
| 233B | R¹ is Et, A = NH, R³ is OC(═O)Oc-Pr |
| | and R⁴ is OMe. |
| 234B | R¹ is Et, A = NH, R³ is OC(═O)OPh and |
| | R⁴ is OMe. |
| 235B | R¹ is Et, A = NH, R³ is OC(═O)OBn |
| | and R⁴ is OMe. |
| 236B | R¹ is Et, A and R³ are taken together to |
| | form NC(═O)O and R⁴ is OMe. |
| 237B | R¹ is Et, A and R³ are taken together to |
| | form NCH₂O and R⁴ is OMe. |
| 238B | R¹ is Et, A and R³ are taken together to |
| | form NS(═O)₂O and R⁴ is OMe. |
| 239B | R¹ is Et, A = NH, R³ is SMe and R⁴ is |
| | OMe. |
| 240B | R¹ is Et, A = NH, R³ is S(═O)Me and |
| | R⁴ is OMe. |
| 241B | R¹ is Et, A = NH, R³ is S(═O)₂Me and |
| | R⁴ is OMe. |
| 242B | R¹ is Et, A = NC(═O)Me, R³ is |
| | OC(═O)Me and R⁴ is OMe. |
| 243B | R¹ is Et, A = NC(═O)Et, R³ is |
| | OC(═O)Et and R⁴ is OMe. |
| 244B | R¹ is Et, A = NC(═O)i-Pr, R³ is |
| | OC(═O)i-Pr and R⁴ is OMe. |
| 245B | R¹ is Et, A = NC(═O)Ph, R³ is |
| | OC(═O)Ph and R⁴ is OMe. |
| 246B | R¹ is Et, A = NC(═O)OMe, R³ is |
| | OC(═O)OMe and R⁴ is OMe. |
| 247B | R¹ is Pr, A = NH, R³ is Cl and R⁴ is H. |
| 248B | R¹ is Pr, A = NH, R³ is Br and R⁴ is H. |
| 249B | R¹ is Pr, A = NH, R³ is OS(═O)₂Me and |
| | R⁴ is H. |
| 250B | R¹ is Pr, A = NH, R³ is OS(═O)₂Et and |
| | R⁴ is H. |
| 251B | R¹ is Pr, A = NH, R³ is OS(═O)₂Pr and |
| | R⁴ is H. |
| 252B | R¹ is Pr, A = NH, R³ is OS(═O)₂i-Pr |
| | and R⁴ is H. |
| 253B | R¹ is Pr, A = NH, R³ is OS(═O)₂Bu and |
| | R⁴ is H. |
| 254B | R¹ is Pr, A = NH, R³ is OS(═O)₂t-Bu |
| | and R⁴ is H. |
| 255B | R¹ is Pr, A = NH, R³ is OS(═O)₂c-Pr |
| | and R⁴ is H. |
| 256B | R¹ is Pr, A = NH, R³ is OS(═O)₂OMe |
| | and R⁴ is H. |
| 257B | R¹ is Pr, A = NH, R³ is OS(═O)₂Ph and |
| | R⁴ is H. |
| 258B | R¹ is Pr, A = NH, R³ is OS(═O)₂Bn and |
| | R⁴ is H. |
| 259B | R¹ is Pr, A = NH, R³ is OC(═O)Me and |
| | R⁴ is H. |
| 260B | R¹ is Pr, A = NH, R³ is OC(═O)Et and |
| | R⁴ is H. |
| 261B | R¹ is Pr, A = NH, R³ is OC(═O)Pr and |
| | R⁴ is H. |
| 262B | R¹ is Pr, A = NH, R³ is OC(═O)i-Pr and |
| | R⁴ is H. |
| 263B | R¹ is Pr, A = NH, R³ is OC(═O)Bu and |
| | R⁴ is H. |
| 264B | R¹ is Pr, A = NH, R³ is OC(═O)t-Bu and |
| | R⁴ is H. |
| 265B | R¹ is Pr, A = NH, R³ is OC(═O)c-Pr and |
| | R⁴ is H. |
| 266B | R¹ is Pr, A = NH, R³ is OC(═O)Ph and |
| | R⁴ is H. |
| 267B | R¹ is Pr, A = NH, R³ is OC(═O)Bn and |
| | R⁴ is H. |
| 268B | R¹ is Pr, A = NH, R³ is OC(═O)OMe |
| | and R⁴ is H. |
| 269B | R¹ is Pr, A = NH, R³ is OC(═O)OEt and |
| | R⁴ is H. |
| 270B | R¹ is Pr, A = NH, R³ is OC(═O)OPr and |
| | R⁴ is H. |
| 271B | R¹ is Pr, A = NH, R³ is OC(═O)Oi-Pr |
| | and R⁴ is H. |
| 272B | R¹ is Pr, A = NH, R³ is OC(═O)OBu |
| | and R⁴ is H. |
| 273B | R¹ is Pr, A = NH, R³ is OC(═O)Ot-Bu |
| | and R⁴ is H. |
| 274B | R¹ is Pr, A = NH, R³ is OC(═O)Oc-Pr |
| | and R⁴ is H. |
| 275B | R¹ is Pr, A = NH, R³ is OC(═O)OPh and |
| | R⁴ is H. |
| 276B | R¹ is Pr, A = NH, R³ is OC(═O)OBn |
| | and R⁴ is H. |
| 277B | R¹ is Pr, A and R³ are taken together to |
| | form NC(═O)O and R⁴ is H. |
| 278B | R¹ is Pr, A and R³ are taken together to |
| | form NCH₂O and R⁴ is H. |
| 279B | R¹ is Pr, A and R³ are taken together to |
| | form NS(═O)₂O and R⁴ is H. |
| 280B | R¹ is Pr, A = NH, R³ is SMe and R⁴ is |
| | H. |
| 281B | R¹ is Pr, A = NH, R³ is S(═O)Me and |
| | R⁴ is H. |
| 282B | R¹ is Pr, A = NH, R³ is S(═O)₂Me and |
| | R⁴ is H. |
| 283B | R¹ is Pr, A = NC(═O)Me, R³ is |
| | OC(═O)Me and R⁴ is H. |
| 284B | R¹ is Pr, A = NC(═O)Et, R³ is |
| | OC(═O)Et and R⁴ is H. |
| 285B | R¹ is Pr, A = NC(═O)i-Pr, R³ is |
| | OC(═O)i-Pr and R⁴ is H. |
| 286B | R¹ is Pr, A = NC(═O)Ph, R³ is |
| | OC(═O)Ph and R⁴ is H. |
| 287B | R¹ is Pr, A = NC(═O)OMe, R³ is |
| | OC(═O)OMe and R⁴ is H. |
| 288B | R¹ is Pr, A = NH, R³ is Cl and R⁴ is Cl. |
| 289B | R¹ is Pr, A = NH, R³ is Br and R⁴ is Cl. |
| 290B | R¹ is Pr, A = NH, R³ is OS(═O)₂Me and |
| | R⁴ is Cl. |
| 291B | R¹ is Pr, A = NH, R³ is OS(═O)₂Et and |
| | R⁴ is Cl. |
| 292B | R¹ is Pr, A = NH, R³ is OS(═O)₂Pr and |
| | R⁴ is Cl. |
| 293B | R¹ is Pr, A = NH, R³ is OS(═O)₂i-Pr |
| | and R⁴ is Cl. |
| 294B | R¹ is Pr, A = NH, R³ is OS(═O)₂Bu and |
| | R⁴ is Cl. |
| 295B | R¹ is Pr, A = NH, R³ is OS(═O)₂t-Bu |
| | and R⁴ is Cl. |
| 296B | R¹ is Pr, A = NH, R³ is OS(═O)₂c-Pr |
| | and R⁴ is Cl. |
| 297B | R¹ is Pr, A = NH, R³ is OS(═O)₂OMe |
| | and R⁴ is Cl. |
| 298B | R¹ is Pr, A = NH, R³ is OS(═O)₂Ph and |
| | R⁴ is Cl. |
| 299B | R¹ is Pr, A = NH, R³ is OS(═O)₂Bn and |
| | R⁴ is Cl. |
| 300B | R¹ is Pr, A = NH, R³ is OC(═O)Me and |
| | R⁴ is Cl. |
| 301B | R¹ is Pr, A = NH, R³ is OC(═O)Et and |
| | R⁴ is Cl. |
| 302B | R¹ is Pr, A = NH, R³ is OC(═O)Pr and |
| | R⁴ is Cl. |
| 303B | R¹ is Pr, A = NH, R³ is OC(═O)i-Pr and |
| | R⁴ is Cl. |
| 304B | R¹ is Pr, A = NH, R³ is OC(═O)Bu and |
| | R⁴ is Cl. |
| 305B | R¹ is Pr, A = NH, R³ is OC(═O)t-Bu and |
| | R⁴ is Cl. |
| 306B | R¹ is Pr, A = NH, R³ is OC(═O)c-Pr and |
| | R⁴ is Cl. |
| 307B | R¹ is Pr, A = NH, R³ is OC(═O)Ph and |
| | R⁴ is Cl. |
| 308B | R¹ is Pr, A = NH, R³ is OC(═O)Bn and |
| | R⁴ is Cl. |
| 309B | R¹ is Pr, A = NH, R³ is OC(═O)OMe |
| | and R⁴ is Cl. |
| 310B | R¹ is Pr, A = NH, R³ is OC(═O)OEt and |
| | R⁴ is Cl. |
| 311B | R¹ is Pr, A = NH, R³ is OC(═O)OPr and |
| | R⁴ is Cl. |
| 312B | R¹ is Pr, A = NH, R³ is OC(═O)Oi-Pr |
| | and R⁴ is Cl. |
| 313B | R¹ is Pr, A = NH, R³ is OC(═O)OBu |
| | and R⁴ is Cl. |
| 314B | R¹ is Pr, A = NH, R³ is OC(═O)Ot-Bu |
| | and R⁴ is Cl. |
| 315B | R¹ is Pr, A = NH, R³ is OC(═O)Oc-Pr |
| | and R⁴ is Cl. |
| 316B | R¹ is Pr, A = NH, R³ is OC(═O)OPh and |
| | R⁴ is Cl. |
| 317B | R¹ is Pr, A = NH, R³ is OC(═O)OBn |
| | and R⁴ is Cl. |
| 318B | R¹ is Pr, A and R³ are taken together to |
| | form NC(═O)O and R⁴ is Cl. |
| 319B | R¹ is Pr, A and R³ are taken together to |
| | form NCH₂O and R⁴ is Cl. |
| 320B | R¹ is Pr, A and R³ are taken together to |
| | form NS(═O)₂O and R⁴ is Cl. |
| 321B | R¹ is Pr, A = NH, R³ is SMe and R⁴ is |
| | Cl. |
| 322B | R¹ is Pr, A = NH, R³ is S(═O)Me and |
| | R⁴ is Cl. |
| 323B | R¹ is Pr, A = NH, R³ is S(═O)₂Me and |
| | R⁴ is Cl. |
| 324B | R¹ is Pr, A = NC(═O)Me, R³ is |
| | OC(═O)Me and R⁴ is Cl. |
| 325B | R¹ is Pr, A = NC(═O)Et, R³ is |
| | OC(═O)Et and R⁴ is Cl. |
| 326B | R¹ is Pr, A = NC(═O)i-Pr, R³ is |
| | OC(═O)i-Pr and R⁴ is Cl. |
| 327B | R¹ is Pr, A = NC(═O)Ph, R³ is |
| | OC(═O)Ph and R⁴ is Cl. |
| 328B | R¹ is Pr, A = NC(═O)OMe, R³ is |
| | OC(═O)OMe and R⁴ is Cl. |
| 329B | R¹ is Pr, A = NH, R³ is Cl and R⁴ is |
| | OMe. |
| 330B | R¹ is Pr, A = NH, R³ is Br and R⁴ is |
| | OMe. |
| 331B | R¹ is Pr, A = NH, R³ is OS(═O)₂Me and |
| | R⁴ is OMe. |
| 332B | R¹ is Pr, A = NH, R³ is OS(═O)₂Et and |
| | R⁴ is OMe. |
| 333B | R¹ is Pr, A = NH, R³ is OS(═O)₂Pr and |
| | R⁴ is OMe. |
| 334B | R¹ is Pr, A = NH, R³ is OS(═O)₂i-Pr |
| | and R⁴ is OMe. |
| 335B | R¹ is Pr, A = NH, R³ is OS(═O)₂Bu and |
| | R⁴ is OMe. |
| 336B | R¹ is Pr, A = NH, R³ is OS(═O)₂t-Bu |
| | and R⁴ is OMe. |
| 337B | R¹ is Pr, A = NH, R³ is OS(═O)₂c-Pr |
| | and R⁴ is OMe. |
| 338B | R¹ is Pr, A = NH, R³ is OS(═O)₂OMe |
| | and R⁴ is OMe. |
| 339B | R¹ is Pr, A = NH, R³ is OS(═O)₂Ph and |
| | R⁴ is OMe. |
| 340B | R¹ is Pr, A = NH, R³ is OS(═O)₂Bn and |
| | R⁴ is OMe. |
| 341B | R¹ is Pr, A = NH, R³ is OC(═O)Me and |
| | R⁴ is OMe. |
| 342B | R¹ is Pr, A = NH, R³ is OC(═O)Et and |
| | R⁴ is OMe. |
| 343B | R¹ is Pr, A = NH, R³ is OC(═O)Pr and |
| | R⁴ is OMe. |
| 344B | R¹ is Pr, A = NH, R³ is OC(═O)i-Pr and |
| | R⁴ is OMe. |
| 345B | R¹ is Pr, A = NH, R³ is OC(═O)Bu and |
| | R⁴ is OMe. |
| 346B | R¹ is Pr, A = NH, R³ is OC(═O)t-Bu and |
| | R⁴ is OMe. |
| 347B | R¹ is Pr, A = NH, R³ is OC(═O)c-Pr and |
| | R⁴ is OMe. |
| 348B | R¹ is Pr, A = NH, R³ is OC(═O)Ph and |
| | R⁴ is OMe. |
| 349B | R¹ is Pr, A = NH, R³ is OC(═O)Bn and |
| | R⁴ is OMe. |
| 350B | R¹ is Pr, A = NH, R³ is OC(═O)OMe |
| | and R⁴ is OMe. |
| 351B | R¹ is Pr, A = NH, R³ is OC(═O)OEt and |
| | R⁴ is OMe. |
| 352B | R¹ is Pr, A = NH, R³ is OC(═O)OPr and |
| | R⁴ is OMe. |
| 353B | R¹ is Pr, A = NH, R³ is OC(═O)Oi-Pr |
| | and R⁴ is OMe. |
| 354B | R¹ is Pr, A = NH, R³ is OC(═O)OBu |
| | and R⁴ is OMe. |
| 355B | R¹ is Pr, A = NH, R³ is OC(═O)Ot-Bu |
| | and R⁴ is OMe. |
| 356B | R¹ is Pr, A = NH, R³ is OC(═O)Oc-Pr |
| | and R⁴ is OMe. |
| 357B | R¹ is Pr, A = NH, R³ is OC(═O)OPh and |
| | R⁴ is OMe. |
| 358B | R¹ is Pr, A = NH, R³ is OC(═O)OBn |
| | and R⁴ is OMe. |
| 359B | R¹ is Pr, A and R³ are taken together to |
| | form NC(═O)O and R⁴ is OMe. |
| 360B | R¹ is Pr, A and R³ are taken together to |
| | form NCH₂O and R⁴ is OMe. |
| 361B | R¹ is Pr, A and R³ are taken together to |
| | form NS(═O)₂O and R⁴ is OMe. |
| 362B | R¹ is Pr, A = NH, R³ is SMe and R⁴ is |
| | OMe. |
| 363B | R¹ is Pr, A = NH, R³ is S(═O)Me and |
| | R⁴ is OMe. |
| 364B | R¹ is Pr, A = NH, R³ is S(═O)₂Me and |
| | R⁴ is OMe. |
| 365B | R¹ is Pr, A = NC(═O)Me, R³ is |
| | OC(═O)Me and R⁴ is OMe. |
| 366B | R¹ is Pr, A = NC(═O)Et, R³ is |
| | OC(═O)Et and R⁴ is OMe. |
| 367B | R¹ is Pr, A = NC(═O)i-Pr, R³ is |
| | OC(═O)i-Pr and R⁴ is OMe. |
| 368B | R¹ is Pr, A = NC(═O)Ph, R³ is |
| | OC(═O)Ph and R⁴ is OMe. |
| 369B | R¹ is Pr, A = NC(═O)OMe, R³ is |
| | OC(═O)OMe and R⁴ is OMe. |
| 370B | R¹ is Bu, A = NH, R³ is Cl and R⁴ is H. |
| 371B | R¹ is Bu, A = NH, R³ is Br and R⁴ is H. |
| 372B | R¹ is Bu, A = NH, R³ is OS(═O)₂Me and R⁴ is |
| | H. |
| 373B | R¹ is Bu, A = NH, R³ is OS(═O)₂Et and R⁴ is |
| | H. |
| 374B | R¹ is Bu, A = NH, R³ is OS(═O)₂Pr and R⁴ is |
| | H. |
| 375B | R¹ is Bu, A = NH, R³ is OS(═O)₂i-Pr and R⁴ is |
| | H. |
| 376B | R¹ is Bu, A = NH, R³ is OS(═O)₂Bu and R⁴ is |
| | H. |
| 377B | R¹ is Bu, A = NH, R³ is OS(═O)₂t-Bu and R⁴ |
| | is H. |
| 378B | R¹ is Bu, A = NH, R³ is OS(═O)₂c-Pr and R⁴ is |
| | H. |
| 379B | R¹ is Bu, A = NH, R³ is OS(═O)₂OMe and R⁴ |
| | is H. |
| 380B | R¹ is Bu, A = NH, R³ is OS(═O)₂Ph and R⁴ is |
| | H. |
| 381B | R¹ is Bu, A = NH, R³ is OS(═O)₂Bn and R⁴ is |
| | H. |
| 382B | R¹ is Bu, A = NH, R³ is OC(═O)Me and R⁴ is |
| | H. |
| 383B | R¹ is Bu, A = NH, R³ is OC(═O)Et and R⁴ is |
| | H. |
| 384B | R¹ is Bu, A = NH, R³ is OC(═O)Pr and R⁴ is |
| | H. |
| 385B | R¹ is Bu, A = NH, R³ is OC(═O)i-Pr and R⁴ is |
| | H. |
| 386B | R¹ is Bu, A = NH, R³ is OC(═O)Bu and R⁴ is |
| | H. |
| 387B | R¹ is Bu, A = NH, R³ is OC(═O)t-Bu and R⁴ is |
| | H. |
| 388B | R¹ is Bu, A = NH, R³ is OC(═O)c-Pr and R⁴ is |
| | H. |
| 389B | R¹ is Bu, A = NH, R³ is OC(═O)Ph and R⁴ is |
| | H. |
| 390B | R¹ is Bu, A = NH, R³ is OC(═O)Bn and R⁴ is |
| | H. |
| 391B | R¹ is Bu, A = NH, R³ is OC(═O)OMe and R⁴ is |
| | H. |
| 392B | R¹ is Bu, A = NH, R³ is OC(═O)OEt and R⁴ is |
| | H. |
| 393B | R¹ is Bu, A = NH, R³ is OC(═O)OPr and R⁴ is |
| | H. |
| 394B | R¹ is Bu, A = NH, R³ is OC(═O)Oi-Pr and R⁴ |
| | is H. |
| 395B | R¹ is Bu, A = NH, R³ is OC(═O)OBu and R⁴ is |
| | H. |
| 396B | R¹ is Bu, A = NH, R³ is OC(═O)Ot-Bu and R⁴ |
| | is H. |
| 397B | R¹ is Bu, A = NH, R³ is OC(═O)Oc-Pr and R⁴ |
| | is H. |
| 398B | R¹ is Bu, A = NH, R³ is OC(═O)OPh and R⁴ is |
| | H. |
| 399B | R¹ is Bu, A = NH, R³ is OC(═O)OBn and R⁴ is |
| | H. |
| 400B | R¹ is Bu, A and R³ are taken together to form |
| | NC(═O)O and R⁴ is H. |
| 401B | R¹ is Bu, A and R³ are taken together to form |
| | NCH₂O and R⁴ is H. |
| 402B | R¹ is Bu, A and R³ are taken together to form |
| | NS(═O)₂O and R⁴ is H. |
| 403B | R¹ is Bu, A = NH, R³ is SMe and R⁴ is H. |
| 404B | R¹ is Bu, A = NH, R³ is S(═O)Me and R⁴ is H. |
| 405B | R¹ is Bu, A = NH, R³ is S(═O)₂Me and R⁴ is |
| | H. |
| 406B | R¹ is Bu, A = NC(═O)Me, R³ is OC(═O)Me |
| | and R⁴ is H. |
| 407B | R¹ is Bu, A = NC(═O)Et, R³ is OC(═O)Et and |
| | R⁴ is H. |
| 408B | R¹ is Bu, A = NC(═O)i-Pr, R³ is OC(═O)i-Pr |
| | and R⁴ is H. |
| 409B | R¹ is Bu, A = NC(═O)Ph, R³ is OC(═O)Ph and |
| | R⁴ is H. |
| 410B | R¹ is Bu, A = NC(═O)OMe, R³ is OC(═O)OMe |
| | and R⁴ is H. |
| 411B | R¹ is Bu, A = NH, R³ is Cl and R⁴ is Cl. |
| 412B | R¹ is Bu, A = NH, R³ is Br and R⁴ is Cl. |
| 413B | R¹ is Bu, A = NH, R³ is OS(═O)₂Me and R⁴ is |
| | Cl. |
| 414B | R¹ is Bu, A = NH, R³ is OS(═O)₂Et and R⁴ is |
| | Cl. |
| 415B | R¹ is Bu, A = NH, R³ is OS(═O)₂Pr and R⁴ is |
| | Cl. |
| 416B | R¹ is Bu, A = NH, R³ is OS(═O)₂i-Pr and R⁴ is |
| | Cl. |
| 417B | R¹ is Bu, A = NH, R³ is OS(═O)₂Bu and R⁴ is |
| | Cl. |
| 418B | R¹ is Bu, A = NH, R³ is OS(═O)₂t-Bu and R⁴ |
| | is Cl. |
| 419B | R¹ is Bu, A = NH, R³ is OS(═O)₂c-Pr and R⁴ is |
| | Cl. |
| 420B | R¹ is Bu, A = NH, R³ is OS(═O)₂OMe and R⁴ |
| | is Cl. |
| 421B | R¹ is Bu, A = NH, R³ is OS(═O)₂Ph and R⁴ is |
| | Cl. |
| 422B | R¹ is Bu, A = NH, R³ is OS(═O)₂Bn and R⁴ is |
| | Cl. |
| 423B | R¹ is Bu, A = NH, R³ is OC(═O)Me and R⁴ is |
| | Cl. |
| 424B | R¹ is Bu, A = NH, R³ is OC(═O)Et and R⁴ is |
| | Cl. |
| 425B | R¹ is Bu, A = NH, R³ is OC(═O)Pr and R⁴ is |
| | Cl. |
| 426B | R¹ is Bu, A = NH, R³ is OC(═O)i-Pr and R⁴ is |
| | Cl. |
| 427B | R¹ is Bu, A = NH, R³ is OC(═O)Bu and R⁴ is |
| | Cl. |
| 428B | R¹ is Bu, A = NH, R³ is OC(═O)t-Bu and R⁴ is |
| | Cl. |
| 429B | R¹ is Bu, A = NH, R³ is OC(═O)c-Pr and R⁴ is |
| | Cl. |
| 430B | R¹ is Bu, A = NH, R³ is OC(═O)Ph and R⁴ is |
| | Cl. |
| 431B | R¹ is Bu, A = NH, R³ is OC(═O)Bn and R⁴ is |
| | Cl. |
| 432B | R¹ is Bu, A = NH, R³ is OC(═O)OMe and R⁴ is |
| | Cl. |
| 433B | R¹ is Bu, A = NH, R³ is OC(═O)OEt and R⁴ is |
| | Cl. |
| 434B | R¹ is Bu, A = NH, R³ is OC(═O)OPr and R⁴ is |
| | Cl. |
| 435B | R¹ is Bu, A = NH, R³ is OC(═O)Oi-Pr and R⁴ |
| | is Cl. |
| 436B | R¹ is Bu, A = NH, R³ is OC(═O)OBu and R⁴ is |
| | Cl. |
| 437B | R¹ is Bu, A = NH, R³ is OC(═O)Ot-Bu and R⁴ |
| | is Cl. |
| 438B | R¹ is Bu, A = NH, R³ is OC(═O)Oc-Pr and R⁴ |
| | is Cl. |
| 439B | R¹ is Bu, A = NH, R³ is OC(═O)OPh and R⁴ is |
| | Cl. |
| 440B | R¹ is Bu, A = NH, R³ is OC(═O)OBn and R⁴ is |
| | Cl. |
| 441B | R¹ is Bu, A and R³ are taken together to form |
| | NC(═O)O and R⁴ is Cl. |
| 442B | R¹ is Bu, A and R³ are taken together to form |
| | NCH₂O and R⁴ is Cl. |
| 443B | R¹ is Bu, A and R³ are taken together to form |
| | NS(═O)₂O and R⁴ is Cl. |
| 444B | R¹ is Bu, A = NH, R³ is SMe and R⁴ is Cl. |
| 445B | R¹ is Bu, A = NH, R³ is S(═O)Me and R⁴ is Cl. |
| 446B | R¹ is Bu, A = NH, R³ is S(═O)₂Me and R⁴ is |
| | Cl. |
| 447B | R¹ is Bu, A = NC(═O)Me, R³ is OC(═O)Me |
| | and R⁴ is Cl. |
| 448B | R¹ is Bu, A = NC(═O)Et, R³ is OC(═O)Et and |
| | R⁴ is Cl. |
| 449B | R¹ is Bu, A = NC(═O)i-Pr, R³ is OC(═O)i-Pr |
| | and R⁴ is Cl. |
| 450B | R¹ is Bu, A = NC(═O)Ph, R³ is OC(═O)Ph and |
| | R⁴ is Cl. |
| 451B | R¹ is Bu, A = NC(═O)OMe, R³ is OC(═O)OMe |
| | and R⁴ is Cl. |
| 452B | R¹ is Bu, A = NH, R³ is Cl and R⁴ is OMe. |
| 453B | R¹ is Bu, A = NH, R³ is Br and R⁴ is OMe. |
| 454B | R¹ is Bu, A = NH, R³ is OS(═O)₂Me and R⁴ is |
| | OMe. |
| 455B | R¹ is Bu, A = NH, R³ is OS(═O)₂Et and R⁴ is |
| | OMe. |
| 456B | R¹ is Bu, A = NH, R³ is OS(═O)₂Pr and R⁴ is |
| | OMe. |
| 457B | R¹ is Bu, A = NH, R³ is OS(═O)₂i-Pr and R⁴ is |
| | OMe. |
| 458B | R¹ is Bu, A = NH, R³ is OS(═O)₂Bu and R⁴ is |
| | OMe. |
| 459B | R¹ is Bu, A = NH, R³ is OS(═O)₂t-Bu and R⁴ |
| | is OMe. |
| 460B | R¹ is Bu, A = NH, R³ is OS(═O)₂c-Pr and R⁴ is |
| | OMe. |
| 461B | R¹ is Bu, A = NH, R³ is OS(═O)₂OMe and R⁴ |
| | is OMe. |
| 462B | R¹ is Bu, A = NH, R³ is OS(═O)₂Ph and R⁴ is |
| | OMe. |
| 463B | R¹ is Bu, A = NH, R³ is OS(═O)₂Bn and R⁴ is |
| | OMe. |
| 464B | R¹ is Bu, A = NH, R³ is OC(═O)Me and R⁴ is |
| | OMe. |
| 465B | R¹ is Bu, A = NH, R³ is OC(═O)Et and R⁴ is |
| | OMe. |
| 466B | R¹ is Bu, A = NH, R³ is OC(═O)Pr and R⁴ is |
| | OMe. |
| 467B | R¹ is Bu, A = NH, R³ is OC(═O)i-Pr and R⁴ is |
| | OMe. |
| 468B | R¹ is Bu, A = NH, R³ is OC(═O)Bu and R⁴ is |
| | OMe. |
| 469B | R¹ is Bu, A = NH, R³ is OC(═O)t-Bu and R⁴ is |
| | OMe. |
| 470B | R¹ is Bu, A = NH, R³ is OC(═O)c-Pr and R⁴ is |
| | OMe. |
| 471B | R¹ is Bu, A = NH, R³ is OC(═O)Ph and R⁴ is |
| | OMe. |
| 472B | R¹ is Bu, A = NH, R³ is OC(═O)Bn and R⁴ is |
| | OMe. |
| 473B | R¹ is Bu, A = NH, R³ is OC(═O)OMe and R⁴ is |
| | OMe. |
| 474B | R¹ is Bu, A = NH, R³ is OC(═O)OEt and R⁴ is |
| | OMe. |
| 475B | R¹ is Bu, A = NH, R³ is OC(═O)OPr and R⁴ is |
| | OMe. |
| 476B | R¹ is Bu, A = NH, R³ is OC(═O)Oi-Pr and R⁴ |
| | is OMe. |
| 477B | R¹ is Bu, A = NH, R³ is OC(═O)OBu and R⁴ is |
| | OMe. |
| 478B | R¹ is Bu, A = NH, R³ is OC(═O)Ot-Bu and R⁴ |
| | is OMe. |
| 479B | R¹ is Bu, A = NH, R³ is OC(═O)Oc-Pr and R⁴ |
| | is OMe. |
| 480B | R¹ is Bu, A = NH, R³ is OC(═O)OPh and R⁴ is |
| | OMe. |
| 481B | R¹ is Bu, A = NH, R³ is OC(═O)OBn and R⁴ is |
| | OMe. |
| 482B | R¹ is Bu, A and R³ are taken together to form |
| | NC(═O)O and R⁴ is OMe. |
| 483B | R¹ is Bu, A and R³ are taken together to form |
| | NCH₂O and R⁴ is OMe. |
| 484B | R¹ is Bu, A and R³ are taken together to form |
| | NS(═O)₂O and R⁴ is OMe. |
| 485B | R¹ is Bu, A = NH, R³ is SMe and R⁴ is OMe. |
| 486B | R¹ is Bu, A = NH, R³ is S(═O)Me and R⁴ is |
| | OMe. |
| 487B | R¹ is Bu, A = NH, R³ is S(═O)₂Me and R⁴ is |
| | OMe. |
| 488B | R¹ is Bu, A = NC(═O)Me, R³ is OC(═O)Me |
| | and R⁴ is OMe. |
| 489B | R¹ is Bu, A = NC(═O)Et, R³ is OC(═O)Et and |
| | R⁴ is OMe. |
| 490B | R¹ is Bu, A = NC(═O)i-Pr, R³ is OC(═O)i-Pr |
| | and R⁴ is OMe. |
| 491B | R¹ is Bu, A = NC(═O)Ph, R³ is OC(═O)Ph and |
| | R⁴ is OMe. |
| 492B | R¹ is Bu, A = NC(═O)OMe, R³ is OC(═O)OMe |
| | and R⁴ is OMe. |
| 493B | R¹ is Ph, A = NH, R³ is Cl and R⁴ is H. |
| 494B | R¹ is Ph, A = NH, R³ is Br and R⁴ is H. |
| 495B | R¹ is Ph, A = NH, R³ is OS(═O)₂Me and R⁴ is |
| | H. |
| 496B | R¹ is Ph, A = NH, R³ is OS(═O)₂Et and R⁴ is |
| | H. |
| 497B | R¹ is Ph, A = NH, R³ is OS(═O)₂Pr and R⁴ is |
| | H. |
| 498B | R¹ is Ph, A = NH, R³ is OS(═O)₂i-Pr and R⁴ is |
| | H. |
| 499B | R¹ is Ph, A = NH, R³ is OS(═O)₂Bu and R⁴ is |
| | H. |
| 500B | R¹ is Ph, A = NH, R³ is OS(═O)₂t-Bu and R⁴ |
| | is H. |
| 501B | R¹ is Ph, A = NH, R³ is OS(═O)₂c-Pr and R⁴ is |
| | H. |
| 502B | R¹ is Ph, A = NH, R³ is OS(═O)₂OMe and R⁴ |
| | is H. |
| 503B | R¹ is Ph, A = NH, R³ is OS(═O)₂Ph and R⁴ is |
| | H. |
| 504B | R¹ is Ph, A = NH, R³ is OS(═O)₂Bn and R⁴ is |
| | H. |
| 505B | R¹ is Ph, A = NH, R³ is OC(═O)Me and R⁴ is |
| | H. |
| 506B | R¹ is Ph, A = NH, R³ is OC(═O)Et and R⁴ is H. |
| 507B | R¹ is Ph, A = NH, R³ is OC(═O)Pr and R⁴ is H. |
| 508B | R¹ is Ph, A = NH, R³ is OC(═O)i-Pr and R⁴ is |
| | H. |
| 509B | R¹ is Ph, A = NH, R³ is OC(═O)Bu and R⁴ is |
| | H. |
| 510B | R¹ is Ph, A = NH, R³ is OC(═O)t-Bu and R⁴ is |
| | H. |
| 511B | R¹ is Ph, A = NH, R³ is OC(═O)c-Pr and R⁴ is |
| | H. |
| 512B | R¹ is Ph, A = NH, R³ is OC(═O)Ph and R⁴ is |
| | H. |
| 513B | R¹ is Ph, A = NH, R³ is OC(═O)Bn and R⁴ is |
| | H. |
| 514B | R¹ is Ph, A = NH, R³ is OC(═O)OMe and R⁴ is |
| | H. |
| 515B | R¹ is Ph, A = NH, R³ is OC(═O)OEt and R⁴ is |
| | H. |
| 516B | R¹ is Ph, A = NH, R³ is OC(═O)OPr and R⁴ is |
| | H. |
| 517B | R¹ is Ph, A = NH, R³ is OC(═O)Oi-Pr and R⁴ |
| | is H. |
| 518B | R¹ is Ph, A = NH, R³ is OC(═O)OBu and R⁴ is |
| | H. |
| 519B | R¹ is Ph, A = NH, R³ is OC(═O)Ot-Bu and R⁴ |
| | is H. |
| 520B | R¹ is Ph, A = NH, R³ is OC(═O)Oc-Pr and R⁴ |
| | is H. |
| 521B | R¹ is Ph, A = NH, R³ is OC(═O)OPh and R⁴ is |
| | H. |
| 522B | R¹ is Ph, A = NH, R³ is OC(═O)OBn and R⁴ is |
| | H. |
| 523B | R¹ is Ph, A and R³ are taken together to form |
| | NC(═O)O and R⁴ is H. |
| 524B | R¹ is Ph, A and R³ are taken together to form |
| | NCH₂O and R⁴ is H. |
| 525B | R¹ is Ph, A and R³ are taken together to form |
| | NS(═O)₂O and R⁴ is H. |
| 526B | R¹ is Ph, A = NH, R³ is SMe and R⁴ is H. |
| 527B | R¹ is Ph, A = NH, R³ is S(═O)Me and R⁴ is H. |
| 528B | R¹ is Ph, A = NH, R³ is S(═O)₂Me and R⁴ is |
| | H. |
| 529B | R¹ is Ph, A = NC(═O)Me, R³ is OC(═O)Me |
| | and R⁴ is H. |
| 530B | R¹ is Ph, A = NC(═O)Et, R³ is OC(═O)Et and |
| | R⁴ is H. |
| 531B | R¹ is Ph, A = NC(═O)i-Pr, R³ is OC(═O)i-Pr |
| | and R⁴ is H. |
| 532B | R¹ is Ph, A = NC(═O)Ph, R³ is OC(═O)Ph and |
| | R⁴ is H. |
| 533B | R¹ is Ph, A = NC(═O)OMe, R³ is OC(═O)OMe |
| | and R⁴ is H. |
| 534B | R¹ is Ph, A = NH, R³ is Cl and R⁴ is Cl. |
| 535B | R¹ is Ph, A = NH, R³ is Br and R⁴ is Cl. |
| 536B | R¹ is Ph, A = NH, R³ is OS(═O)₂Me and R⁴ is |
| | Cl. |
| 537B | R¹ is Ph, A = NH, R³ is OS(═O)₂Et and R⁴ is |
| | Cl. |
| 538B | R¹ is Ph, A = NH, R³ is OS(═O)₂Pr and R⁴ is |
| | Cl. |
| 539B | R¹ is Ph, A = NH, R³ is OS(═O)₂i-Pr and R⁴ is |
| | Cl. |
| 540B | R¹ is Ph, A = NH, R³ is OS(═O)₂Bu and R⁴ is |
| | Cl. |
| 541B | R¹ is Ph, A = NH, R³ is OS(═O)₂t-Bu and R⁴ |
| | is Cl. |
| 542B | R¹ is Ph, A = NH, R³ is OS(═O)₂c-Pr and R⁴ is |
| | Cl. |
| 543B | R¹ is Ph, A = NH, R³ is OS(═O)₂OMe and R⁴ |
| | is Cl. |
| 544B | R¹ is Ph, A = NH, R³ is OS(═O)₂Ph and R⁴ is |
| | Cl. |
| 545B | R¹ is Ph, A = NH, R³ is OS(═O)₂Bn and R⁴ is |
| | Cl. |
| 546B | R¹ is Ph, A = NH, R³ is OC(═O)Me and R⁴ is |
| | Cl. |
| 547B | R¹ is Ph, A = NH, R³ is OC(═O)Et and R⁴ is |
| | Cl. |
| 548B | R¹ is Ph, A = NH, R³ is OC(═O)Pr and R⁴ is |
| | Cl. |
| 549B | R¹ is Ph, A = NH, R³ is OC(═O)i-Pr and R⁴ is |
| | Cl. |
| 550B | R¹ is Ph, A = NH, R³ is OC(═O)Bu and R⁴ is |
| | Cl. |
| 551B | R¹ is Ph, A = NH, R³ is OC(═O)t-Bu and R⁴ is |
| | Cl. |
| 552B | R¹ is Ph, A = NH, R³ is OC(═O)c-Pr and R⁴ is |
| | Cl. |
| 553B | R¹ is Ph, A = NH, R³ is OC(═O)Ph and R⁴ is |
| | Cl. |
| 554B | R¹ is Ph, A = NH, R³ is OC(═O)Bn and R⁴ is |
| | Cl. |
| 555B | R¹ is Ph, A = NH, R³ is OC(═O)OMe and R⁴ is |
| | Cl. |
| 556B | R¹ is Ph, A = NH, R³ is OC(═O)OEt and R⁴ is |
| | Cl. |
| 557B | R¹ is Ph, A = NH, R³ is OC(═O)OPr and R⁴ is |
| | Cl. |
| 558B | R¹ is Ph, A = NH, R³ is OC(═O)Oi-Pr and R⁴ |
| | is Cl. |
| 559B | R¹ is Ph, A = NH, R³ is OC(═O)OBu and R⁴ is |
| | Cl. |
| 560B | R¹ is Ph, A = NH, R³ is OC(═O)Ot-Bu and R⁴ |
| | is Cl. |
| 561B | R¹ is Ph, A = NH, R³ is OC(═O)Oc-Pr and R⁴ |
| | is Cl. |
| 562B | R¹ is Ph, A = NH, R³ is OC(═O)OPh and R⁴ is |
| | Cl. |
| 563B | R¹ is Ph, A = NH, R³ is OC(═O)OBn and R⁴ is |
| | Cl. |
| 564B | R¹ is Ph, A and R³ are taken together to form |
| | NC(═O)O and R⁴ is Cl. |
| 565B | R¹ is Ph, A and R³ are taken together to form |
| | NCH₂O and R⁴ is Cl. |
| 566B | R¹ is Ph, A and R³ are taken together to form |
| | NS(═O)₂O and R⁴ is Cl. |
| 567B | R¹ is Ph, A = NH, R³ is SMe and R⁴ is Cl. |
| 568B | R¹ is Ph, A = NH, R³ is S(═O)Me and R⁴ is Cl. |
| 569B | R¹ is Ph, A = NH, R³ is S(═O)₂Me and R⁴ is |
| | Cl. |
| 570B | R¹ is Ph, A = NC(═O)Me, R³ is OC(═O)Me |
| | and R⁴ is Cl. |
| 571B | R¹ is Ph, A = NC(═O)Et, R³ is OC(═O)Et and |
| | R⁴ is Cl. |
| 572B | R¹ is Ph, A = NC(═O)i-Pr, R³ is OC(═O)i-Pr |
| | and R⁴ is Cl. |
| 573B | R¹ is Ph, A = NC(═O)Ph, R³ is OC(═O)Ph and |
| | R⁴ is Cl. |
| 574B | R¹ is Ph, A = NC(═O)OMe, R³ is OC(═O)OMe |
| | and R⁴ is Cl. |
| 575B | R¹ is Ph, A = NH, R³ is Cl and R⁴ is OMe. |
| 576B | R¹ is Ph, A = NH, R³ is Br and R⁴ is OMe. |
| 577B | R¹ is Ph, A = NH, R³ is OS(═O)₂Me and R⁴ is |
| | OMe. |
| 578B | R¹ is Ph, A = NH, R³ is OS(═O)₂Et and R⁴ is |
| | OMe. |
| 579B | R¹ is Ph, A = NH, R³ is OS(═O)₂Pr and R⁴ is |
| | OMe. |
| 580B | R¹ is Ph, A = NH, R³ is OS(═O)₂i-Pr and R⁴ is |
| | OMe. |
| 581B | R¹ is Ph, A = NH, R³ is OS(═O)₂Bu and R⁴ is |
| | OMe. |
| 582B | R¹ is Ph, A = NH, R³ is OS(═O)₂t-Bu and R⁴ |
| | is OMe. |
| 583B | R¹ is Ph, A = NH, R³ is OS(═O)₂c-Pr and R⁴ is |
| | OMe. |
| 584B | R¹ is Ph, A = NH, R³ is OS(═O)₂OMe and R⁴ |
| | is OMe. |
| 585B | R¹ is Ph, A = NH, R³ is OS(═O)₂Ph and R⁴ is |
| | OMe. |
| 586B | R¹ is Ph, A = NH, R³ is OS(═O)₂Bn and R⁴ is |
| | OMe. |
| 587B | R¹ is Ph, A = NH, R³ is OC(═O)Me and R⁴ is |
| | OMe. |
| 588B | R¹ is Ph, A = NH, R³ is OC(═O)Et and R⁴ is |
| | OMe. |
| 589B | R¹ is Ph, A = NH, R³ is OC(═O)Pr and R⁴ is |
| | OMe. |
| 590B | R¹ is Ph, A = NH, R³ is OC(═O)i-Pr and R⁴ is |
| | OMe. |
| 591B | R¹ is Ph, A = NH, R³ is OC(═O)Bu and R⁴ is |
| | OMe. |
| 592B | R¹ is Ph, A = NH, R³ is OC(═O)t-Bu and R⁴ is |
| | OMe. |
| 593B | R¹ is Ph, A = NH, R³ is OC(═O)c-Pr and R⁴ is |
| | OMe. |
| 594B | R¹ is Ph, A = NH, R³ is OC(═O)Ph and R⁴ is |
| | OMe. |
| 595B | R¹ is Ph, A = NH, R³ is OC(═O)Bn and R⁴ is |
| | OMe. |
| 596B | R¹ is Ph, A = NH, R³ is OC(═O)OMe and R⁴ is |
| | OMe. |
| 597B | R¹ is Ph, A = NH, R³ is OC(═O)OEt and R⁴ is |
| | OMe. |
| 598B | R¹ is Ph, A = NH, R³ is OC(═O)OPr and R⁴ is |
| | OMe. |
| 599B | R¹ is Ph, A = NH, R³ is OC(═O)Oi-Pr and R⁴ |
| | is OMe. |
| 600B | R¹ is Ph, A = NH, R³ is OC(═O)OBu and R⁴ is |
| | OMe. |
| 601B | R¹ is Ph, A = NH, R³ is OC(═O)Ot-Bu and R⁴ |
| | is OMe. |
| 602B | R¹ is Ph, A = NH, R³ is OC(═O)Oc-Pr and R⁴ |
| | is OMe. |
| 603B | R¹ is Ph, A = NH, R³ is OC(═O)OPh and R⁴ is |
| | OMe. |
| 604B | R¹ is Ph, A = NH, R³ is OC(═O)OBn and R⁴ is |
| | OMe. |
| 605B | R¹ is Ph, A and R³ are taken together to form |
| | NC(═O)O and R⁴ is OMe. |
| 606B | R¹ is Ph, A and R³ are taken together to form |
| | NCH₂O and R⁴ is OMe. |
| 607B | R¹ is Ph, A and R³ are taken together to form |
| | NS(═O)₂O and R⁴ is OMe. |
| 608B | R¹ is Ph, A = NH, R³ is SMe and R⁴ is OMe. |
| 609B | R¹ is Ph, A = NH, R³ is S(═O)Me and R⁴ is |
| | OMe. |
| 610B | R¹ is Ph, A = NH, R³ is S(═O)₂Me and R⁴ is |
| | OMe. |
| 611B | R¹ is Ph, A = NC(═O)Me, R³ is OC(═O)Me |
| | and R⁴ is OMe. |
| 612B | R¹ is Ph, A = NC(═O)Et, R³ is OC(═O)Et and |
| | R⁴ is OMe. |
| 613B | R¹ is Ph, A = NC(═O)i-Pr, R³ is OC(═O)i-Pr |
| | and R⁴ is OMe. |
| 614B | R¹ is Ph, A = NC(═O)Ph, R³ is OC(═O)Ph and |
| | R⁴ is OMe. |
| 615B | R¹ is Ph, A = NC(═O)OMe, R³ is OC(═O)OMe |
| | and R⁴ is OMe. |
| 616B | R¹ is i-Pr, A = NH, R³ is Cl and R⁴ is H. |
| 617B | R¹ is i-Pr, A = NH, R³ is Br and R⁴ is H. |
| 618B | R¹ is i-Pr, A = NH, R³ is OS(═O)₂Me and R⁴ is |
| | H. |
| 619B | R¹ is i-Pr, A = NH, R³ is OS(═O)₂Et and R⁴ is |
| | H. |
| 620B | R¹ is i-Pr, A = NH, R³ is OS(═O)₂Pr and R⁴ is |
| | H. |
| 621B | R¹ is i-Pr, A = NH, R³ is OS(═O)₂i-Pr and R⁴ |
| | is H. |
| 622B | R¹ is i-Pr, A = NH, R³ is OS(═O)₂Bu and R⁴ is |
| | H. |
| 623B | R¹ is i-Pr, A = NH, R³ is OS(═O)₂t-Bu and R⁴ |
| | is H. |
| 624B | R¹ is i-Pr, A = NH, R³ is OS(═O)₂c-Pr and R⁴ |
| | is H. |
| 625B | R¹ is i-Pr, A = NH, R³ is OS(═O)₂OMe and R⁴ |
| | is H. |
| 626B | R¹ is i-Pr, A = NH, R³ is OS(═O)₂Ph and R⁴ is |
| | H. |
| 627B | R¹ is i-Pr, A = NH, R³ is OS(═O)₂Bn and R⁴ is |
| | H. |
| 628B | R¹ is i-Pr, A = NH, R³ is OC(═O)Me and R⁴ is |
| | H. |
| 629B | R¹ is i-Pr, A = NH, R³ is OC(═O)Et and R⁴ is |
| | H. |
| 630B | R¹ is i-Pr, A = NH, R³ is OC(═O)Pr and R⁴ is |
| | H. |
| 631B | R¹ is i-Pr, A = NH, R³ is OC(═O)i-Pr and R⁴ is |
| | H. |
| 632B | R¹ is i-Pr, A = NH, R³ is OC(═O)Bu and R⁴ is |
| | H. |
| 633B | R¹ is i-Pr, A = NH, R³ is OC(═O)t-Bu and R⁴ |
| | is H. |
| 634B | R¹ is i-Pr, A = NH, R³ is OC(═O)c-Pr and R⁴ is |
| | H. |
| 635B | R¹ is i-Pr, A = NH, R³ is OC(═O)Ph and R⁴ is |
| | H. |
| 636B | R¹ is i-Pr, A = NH, R³ is OC(═O)Bn and R⁴ is |
| | H. |
| 637B | R¹ is i-Pr, A = NH, R³ is OC(═O)OMe and R⁴ |
| | is H. |
| 638B | R¹ is i-Pr, A = NH, R³ is OC(═O)OEt and R⁴ is |
| | H. |
| 639B | R¹ is i-Pr, A = NH, R³ is OC(═O)OPr and R⁴ is |
| | H. |
| 640B | R¹ is i-Pr, A = NH, R³ is OC(═O)Oi-Pr and R⁴ |
| | is H. |
| 641B | R¹ is i-Pr, A = NH, R³ is OC(═O)OBu and R⁴ |
| | is H. |
| 642B | R¹ is i-Pr, A = NH, R³ is OC(═O)Ot-Bu and R⁴ |
| | is H. |
| 643B | R¹ is i-Pr, A = NH, R³ is OC(═O)Oc-Pr and R⁴ |
| | is H. |
| 644B | R¹ is i-Pr, A = NH, R³ is OC(═O)OPh and R⁴ |
| | is H. |
| 645B | R¹ is i-Pr, A = NH, R³ is OC(═O)OBn and R⁴ |
| | is H. |
| 646B | R¹ is i-Pr, A and R³ are taken together to form |
| | NC(═O)O and R⁴ is H. |
| 647B | R¹ is i-Pr, A and R³ are taken together to form |
| | NCH₂O and R⁴ is H. |
| 648B | R¹ is i-Pr, A and R³ are taken together to form |
| | NS(═O)₂O and R⁴ is H. |
| 649B | R¹ is i-Pr, A = NH, R³ is SMe and R⁴ is H. |
| 650B | R¹ is i-Pr, A = NH, R³ is S(═O)Me and R⁴ is H. |
| 651B | R¹ is i-Pr, A = NH, R³ is S(═O)₂Me and R⁴ is |
| | H. |
| 652B | R¹ is i-Pr, A = NC(═O)Me, R³ is OC(═O)Me |
| | and R⁴ is H. |
| 653B | R¹ is i-Pr, A = NC(═O)Et, R³ is OC(═O)Et and |
| | R⁴ is H. |
| 654B | R¹ is i-Pr, A = NC(═O)i-Pr, R³ is OC(═O)i-Pr |
| | and R⁴ is H. |
| 655B | R¹ is i-Pr, A = NC(═O)Ph, R³ is OC(═O)Ph and |
| | R⁴ is H. |
| 656B | R¹ is i-Pr, A = NC(═O)OMe, R³ is |
| | OC(═O)OMe and R⁴ is H. |
| 657B | R¹ is i-Pr, A = NH, R³ is Cl and R⁴ is Cl. |
| 658B | R¹ is i-Pr, A = NH, R³ is Br and R⁴ is Cl. |
| 659B | R¹ is i-Pr, A = NH, R³ is OS(═O)₂Me and R⁴ is |
| | Cl. |
| 660B | R¹ is i-Pr, A = NH, R³ is OS(═O)₂Et and R⁴ is |
| | Cl. |
| 661B | R¹ is i-Pr, A = NH, R³ is OS(═O)₂Pr and R⁴ is |
| | Cl. |
| 662B | R¹ is i-Pr, A = NH, R³ is OS(═O)₂i-Pr and R⁴ |
| | is Cl. |
| 663B | R¹ is i-Pr, A = NH, R³ is OS(═O)₂Bu and R⁴ is |
| | Cl. |
| 664B | R¹ is i-Pr, A = NH, R³ is OS(═O)₂t-Bu and R⁴ |
| | is Cl. |
| 665B | R¹ is i-Pr, A = NH, R³ is OS(═O)₂c-Pr and R⁴ |
| | is Cl. |
| 666B | R¹ is i-Pr, A = NH, R³ is OS(═O)₂OMe and R⁴ |
| | is Cl. |
| 667B | R¹ is i-Pr, A = NH, R³ is OS(═O)₂Ph and R⁴ is |
| | Cl. |
| 668B | R¹ is i-Pr, A = NH, R³ is OS(═O)₂Bn and R⁴ is |
| | Cl. |
| 669B | R¹ is i-Pr, A = NH, R³ is OC(═O)Me and R⁴ is |
| | Cl. |
| 670B | R¹ is i-Pr, A = NH, R³ is OC(═O)Et and R⁴ is |
| | Cl. |
| 671B | R¹ is i-Pr, A = NH, R³ is OC(═O)Pr and R⁴ is |
| | Cl. |
| 672B | R¹ is i-Pr, A = NH, R³ is OC(═O)i-Pr and R⁴ is |
| | Cl. |
| 673B | R¹ is i-Pr, A = NH, R³ is OC(═O)Bu and R⁴ is |
| | Cl. |
| 674B | R¹ is i-Pr, A = NH, R³ is OC(═O)t-Bu and R⁴ |
| | is Cl. |
| 675B | R¹ is i-Pr, A = NH, R³ is OC(═O)c-Pr and R⁴ is |
| | Cl. |
| 676B | R¹ is i-Pr, A = NH, R³ is OC(═O)Ph and R⁴ is |
| | Cl. |
| 677B | R¹ is i-Pr, A = NH, R³ is OC(═O)Bn and R⁴ is |
| | Cl. |
| 678B | R¹ is i-Pr, A = NH, R³ is OC(═O)OMe and R⁴ |
| | is Cl. |
| 679B | R¹ is i-Pr, A = NH, R³ is OC(═O)OEt and R⁴ is |
| | Cl. |
| 680B | R¹ is i-Pr, A = NH, R³ is OC(═O)OPr and R⁴ is |
| | Cl. |
| 681B | R¹ is i-Pr, A = NH, R³ is OC(═O)Oi-Pr and R⁴ |
| | is Cl. |
| 682B | R¹ is i-Pr, A = NH, R³ is OC(═O)OBu and R⁴ |
| | is Cl. |
| 683B | R¹ is i-Pr, A = NH, R³ is OC(═O)Ot-Bu and R⁴ |
| | is Cl. |
| 684B | R¹ is i-Pr, A = NH, R³ is OC(═O)Oc-Pr and R⁴ |
| | is Cl. |
| 685B | R¹ is i-Pr, A = NH, R³ is OC(═O)OPh and R⁴ |
| | is Cl. |
| 686B | R¹ is i-Pr, A = NH, R³ is OC(═O)OBn and R⁴ |
| | is Cl. |
| 687B | R¹ is i-Pr, A and R³ are taken together to form |
| | NC(═O)O and R⁴ is Cl. |
| 688B | R¹ is i-Pr, A and R³ are taken together to form |
| | NCH₂O and R⁴ is Cl. |
| 689B | R¹ is i-Pr, A and R³ are taken together to form |
| | NS(═O)₂O and R⁴ is Cl. |
| 690B | R¹ is i-Pr, A = NH, R³ is SMe and R⁴ is Cl. |
| 691B | R¹ is i-Pr, A = NH, R³ is S(═O)Me and R⁴ is |
| | Cl. |
| 692B | R¹ is i-Pr, A = NH, R³ is S(═O)₂Me and R⁴ is |
| | Cl. |
| 693B | R¹ is i-Pr, A = NC(═O)Me, R³ is OC(═O)Me |
| | and R⁴ is Cl. |
| 694B | R¹ is i-Pr, A = NC(═O)Et, R³ is OC(═O)Et and |
| | R⁴ is Cl. |
| 695B | R¹ is i-Pr, A = NC(═O)i-Pr, R³ is OC(═O)i-Pr |
| | and R⁴ is Cl. |
| 696B | R¹ is i-Pr, A = NC(═O)Ph, R³ is OC(═O)Ph and |
| | R⁴ is Cl. |
| 697B | R¹ is i-Pr, A = NC(═O)OMe, R³ is |
| | OC(═O)OMe and R⁴ is Cl. |
| 698B | R¹ is i-Pr, A = NH, R³ is Cl and R⁴ is OMe. |
| 699B | R¹ is i-Pr, A = NH, R³ is Br and R⁴ is OMe. |
| 700B | R¹ is i-Pr, A = NH, R³ is OS(═O)₂Me and R⁴ is |
| | OMe. |
| 701B | R¹ is i-Pr, A = NH, R³ is OS(═O)₂Et and R⁴ is |
| | OMe. |
| 702B | R¹ is i-Pr, A = NH, R³ is OS(═O)₂Pr and R⁴ is |
| | OMe. |
| 703B | R¹ is i-Pr, A = NH, R³ is OS(═O)₂i-Pr and R⁴ |
| | is OMe. |
| 704B | R¹ is i-Pr, A = NH, R³ is OS(═O)₂Bu and R⁴ is |
| | OMe. |
| 705B | R¹ is i-Pr, A = NH, R³ is OS(═O)₂t-Bu and R⁴ |
| | is OMe. |
| 706B | R¹ is i-Pr, A = NH, R³ is OS(═O)₂c-Pr and R⁴ |
| | is OMe. |
| 707B | R¹ is i-Pr, A = NH, R³ is OS(═O)₂OMe and R⁴ |
| | is OMe. |
| 708B | R¹ is i-Pr, A = NH, R³ is OS(═O)₂Ph and R⁴ is |
| | OMe. |
| 709B | R¹ is i-Pr, A = NH, R³ is OS(═O)₂Bn and R⁴ is |
| | OMe. |
| 710B | R¹ is i-Pr, A = NH, R³ is OC(═O)Me and R⁴ is |
| | OMe. |
| 711B | R¹ is i-Pr, A = NH, R³ is OC(═O)Et and R⁴ is |
| | OMe. |
| 712B | R¹ is i-Pr, A = NH, R³ is OC(═O)Pr and R⁴ is |
| | OMe. |
| 713B | R¹ is i-Pr, A = NH, R³ is OC(═O)i-Pr and R⁴ is |
| | OMe. |
| 714B | R¹ is i-Pr, A = NH, R³ is OC(═O)Bu and R⁴ is |
| | OMe. |
| 715B | R¹ is i-Pr, A = NH, R³ is OC(═O)t-Bu and R⁴ is |
| | OMe. |
| 716B | R¹ is i-Pr, A = NH, R³ is OC(═O)c-Pr and R⁴ is |
| | OMe. |
| 717B | R¹ is i-Pr, A = NH, R³ is OC(═O)Ph and R⁴ is |
| | OMe. |
| 718B | R¹ is i-Pr, A = NH, R³ is OC(═O)Bn and R⁴ is |
| | OMe. |
| 719B | R¹ is i-Pr, A = NH, R³ is OC(═O)OMe and R⁴ |
| | is OMe. |
| 720B | R¹ is i-Pr, A = NH, R³ is OC(═O)OEt and R⁴ is |
| | OMe. |
| 721B | R¹ is i-Pr, A = NH, R³ is OC(═O)OPr and R⁴ is |
| | OMe. |
| 722B | R¹ is i-Pr, A = NH, R³ is OC(═O)Oi-Pr and R⁴ |
| | is OMe. |
| 723B | R¹ is i-Pr, A = NH, R³ is OC(═O)OBu and R⁴ |
| | is OMe. |
| 724B | R¹ is i-Pr, A = NH, R³ is OC(═O)Ot-Bu and R⁴ |
| | is OMe. |
| 725B | R¹ is i-Pr, A = NH, R³ is OC(═O)Oc-Pr and R⁴ |
| | is OMe. |
| 726B | R¹ is i-Pr, A = NH, R³ is OC(═O)OPh and R⁴ |
| | is OMe. |
| 727B | R¹ is i-Pr, A = NH, R³ is OC(═O)OBn and R⁴ |
| | is OMe. |
| 728B | R¹ is i-Pr, A and R³ are taken together to form |
| | NC(═O)O and R⁴ is OMe. |
| 729B | R¹ is i-Pr, A and R³ are taken together to form |
| | NCH₂O and R⁴ is OMe. |
| 730B | R¹ is i-Pr, A and R³ are taken together to form |
| | NS(═O)₂O and R⁴ is OMe. |
| 731B | R¹ is i-Pr, A = NH, R³ is SMe and R⁴ is OMe. |
| 732B | R¹ is i-Pr, A = NH, R³ is S(═O)Me and R⁴ is |
| | OMe. |
| 733B | R¹ is i-Pr, A = NH, R³ is S(═O)₂Me and R⁴ is |
| | OMe. |
| 734B | R¹ is i-Pr, A = NC(═O)Me, R³ is OC(═O)Me |
| | and R⁴ is OMe. |
| 735B | R¹ is i-Pr, A = NC(═O)Et, R³ is OC(═O)Et and |
| | R⁴ is OMe. |
| 736B | R¹ is i-Pr, A = NC(═O)i-Pr, R³ is OC(═O)i-Pr |
| | and R⁴ is OMe. |
| 737B | R¹ is i-Pr, A = NC(═O)Ph, R³ is OC(═O)Ph and |
| | R⁴ is OMe. |
| 738B | R¹ is i-Pr, A = NC(═O)OMe, R³ is |
| | OC(═O)OMe and R⁴ is OMe. |

### Formulation/Utility

A compound of this invention will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively, the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight except where otherwise indicated and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be constructed as merely illustrative, and not limiting of the disclosure in any way whatsoever.

### Example A

| High Strength Concentrate | |
|---|---|
| Compound 2 | 98.5% |
| silica aerogel | 0.5% |
| synthetic amorphous fine silica | 1.0% |

### Example B

| Wettable Powder | |
|---|---|
| Compound 2 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

| Granule | |
|---|---|
| Compound 2 | 10.0% |
| attapulgite granules (low volatile matter, | 90.0% |
| 0.71/0.30 mm; U.S.S. No. 25-50 sieves) | |

### Example D

| Extruded Pellet | |
|---|---|
| Compound 2 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

| Emulsifiable Concentrate | |
|---|---|
| Compound 2 | 10.0% |
| polyoxyethylene sorbitol hexoleate | 20.0% |
| C₆-C₁₀ fatty acid methyl ester | 70.0% |

### Example F

| Microemulsion | |
|---|---|
| Compound 2 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

| Seed Treatment | |
|---|---|
| Compound 2 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

The present disclosure also includes Examples A through G above except “Compound 2” is replaced with “Compound 21”, “Compound 47”, “Compound 50”, “Compound 75”, “Compound 90”, “Compound 91”, “Compound 96”, “Compound 97”, “Compound 98” and “Compound 99”.

Water-soluble and water-dispersible formulations are typically diluted with water to form aqueous compositions before application. Aqueous compositions for direct applications to the plant or portion thereof (e.g., spray tank compositions) typically at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuliginea, Podosphaera leucotricha and Pseudocercosporella herpotrichoides, Botrytis diseases such as Botrytis cinerea, Monilinia fructicola, Sclerotinia diseases such as Sclerotinia sclerotiorum, Sclerotinia minor, Magnaporthe grisea, and Phomopsis viticola, Helminthosporium diseases such as Helminthosporium tritici repentis and Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondita, Puccinia striiformis, Puccinia hordei, Puccinia graminis and Puccinia arachidis), Hemileia vastatrix and Phakopsora pachyrhizi; other pathogens including Rutstroemia floccosum (also known as Sclerotinia homoeocarpa); Rhizoctonia spp. (such as Rhizoctonia solani); Fusarium diseases such as Fusarium roseum, Fusarium graminearum and Fusarium oxysporum Verticillium dahliae; Sclerotium rolfsii; Rynchosporium secalis; Cercosporidium personatum, Cercospora arachidicola and Cercospora beticola; Rhizopus spp. (such as Rhizopus stolonifer); Aspergillus spp. (such as Aspergillus flavus and Aspergillus parasiticus); and other genera and species closely related to these pathogens. In addition to their fungicidal activity, the compositions or combinations also have activity against bacteria such as Erwinia amylovora, Xanthomonas campestris, Pseudomonas syringae, and other related species. Furthermore, the compounds of this invention are useful in treating postharvest diseases of fruits and vegetables caused by fungi and bacteria. These infections can occur before, during and after harvest. For example, infections can occur before harvest and then remain dormant until some point during ripening (e.g., host begins tissue changes in such a way that infection can progress); also infections can arise from surface wounds created by mechanical or insect injury. In this respect, the compounds of this invention can reduce losses (i.e. losses resulting from quantity and quality) due to postharvest diseases which may occur at any time from harvest to consumption. Treatment of postharvest diseases with compounds of the invention can increase the period of time during which perishable edible plant parts (e.g, fruits, seeds, foliage, stems, bulbs, tubers) can be stored refrigerated or un-refrigerated after harvest, and remain edible and free from noticeable or harmful degradation or contamination by fungi or other microorganisms. Treatment of edible plant parts before or after harvest with compounds of the invention can also decrease the formation of toxic metabolites of fungi or other microorganisms, for example, mycotoxins such as aflatoxins.

Plant disease control is ordinarily accomplished by applying an effective amount of a compound of this invention either pre- or post-infection, to the portion of the plant to be protected such as the roots, stems, foliage, fruits, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants. Control of postharvest pathogens which infect the produce before harvest is typically accomplished by field application of a compound of this invention, and in cases where infection occurs after harvest the compounds can be applied to the harvested crop as dips, sprays, fumigants, treated wraps and box liners.

Rates of application for these compounds (i.e. a fungicidally effective amount) can be influenced by factors such as the plant diseases to be controlled, the plant species to be protected, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10)N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) hetero aromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazoles and thiophanates. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. Demethylation fungicides include piperazines, pyridines, pyrimidines, imidazoles and triazoles. The piperazines include triforine. The pyridines include buthiobate and pyrifenox. The pyrimidines include fenarimol, nuarimol and triarimol. The imidazoles include clotrimazole, imazalil, oxpoconazole, prochloraz, pefurazoate and triflumizole. The triazoles include azaconazole, bitertanol, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, 1-[[(2S,3R)-3-(2-chlorophenyl)-2-(2,4-difluorophenyl)-2-oxiranyl]methyl]-1H-1,2,4-triazole, 2-[[(2S,3R)-3-(2-chlorophenyl)-2-(2,4-difluorophenyl)-2-oxiranyl]methyl]-1,2-dihydro-3H-1,2,4-triazole-3-thione and 1-[[(2S,3R)-3-(2-chlorophenyl)-2-(2,4-difluorophenyl)-2-oxiranyl]methyl]-5-(2-propen-1-ylthio)-1H-1,2,4-triazole. The imidazoles include clotrimazole, imazalil, oxpoconazole, prochloraz, pefurazoate and triflumizole. Biochemical investigations have shown that all of the above mentioned fungicides are DMI fungicides as described by K. H. Kuck et al. in Modern Selective Fungicides-Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanines, oxazolidinones and butyrolactones. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholines, piperidines and spiroketal-amines. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phophorothiolates and dithiolanes. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include phenyl benzamides, pyridinyl ethyl benzamides, furan carboxamides, oxathiin carboxamides, thiazole carboxamides, pyrazole carboxamides and pyridine carboxamides. The phenyl benzamides include benodanil, flutolanil and mepronil. The pyridinyl ethyl benzamides include fluopyram. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, isopyrazam, benzovindiflupyr, N-[2-(1S,2R)-[1,1′-bicyclopropyl]-2-ylphenyl]-3-(difluoromethyl)-1-methyl-1H-pyrazole-4-carboxamide, penflufen, (N-[2-(1,3-dimethylbutyl)phenyl]-5-fluoro-1,3-dimethyl-1H-pyrazole-4-carboxamide), N-[2-(2,4-dichlorophenyl)-2-methoxy-1-methylethyl]-3-(difluoromethyl)-1-methyl-1H-pyrazole-4-carboxamide and N-cyclopropyl-3-(difluoromethyl)-5-fluoro-1-methyl-N-[[2-(1-methylethyl)phenyl]methyl]-1H-pyrazole-4-carboxamide. The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Qₒ) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylates, methoxycarbamates, oximinoacetates, oximinoacetamides, oxazolidinediones, dihydrodioxazines, imidazolinones and benzylcarbamates. The methoxyacrylates include azoxystrobin, coumoxystrobin, enestroburin, flufenoxystrobin, picoxystrobin and pyraoxystrobin. The methoxycarbamates include pyraclostrobin, pyrametostrobin and triclopyricarb. The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]methyl]benzeneacetamide and 2-[[[3-(2,6-dichlorophenyl)-1-methyl-2-propen-1-ylidene]amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb. Class (11) also includes 2-[(2,5-dimethylphenoxy)methyl]-α-methoxy-N-benzeneacetamide.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Azanaphthalene fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Azanaphthalene fungicides include aryloxyquinolines and quinazolinone. The aryloxyquinolines include quinoxyfen and tebufloquin. The quinazolinones include proquinazid.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbons and 1,2,4-thiadiazoles. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranones, pyrroloquinolinones and triazolobenzothiazoles. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamides, carboxamides and propionamides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamates and allylaminess. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazoles and sulfamoyltriazoles. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly. Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability.

Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazoles and isothiazolones. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amides, valinamide carbamates, carbamates and mandelic acid amides. The cinnamic acid amides include dimethomorph and flumorph. The valinamide carbamates include benthiavalicarb, benthiavalicarb-isopropyl, iprovalicarb, valifenalate and valiphenal. The carbamates include tolprocarb. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide.

(44) “Host plant defense induction fungicides” (Fungicide Resistance Action Committee (FRAC) code P) induce host plant defense mechanisms. Host plant defense induction fungicides include benzothiadiazoles, benzisothiazoles and thiadiazolecarboxamides. The benzothiadiazoles include acibenzolar-S-methyl. The benzisothiazoles include probenazole. The thiadiazolecarboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenylacetamide fungicides” (Fungicide Resistance Action Committee (FRAC) code U6), (46.3) “arylphenylketone fungicides” (Fungicide Resistance Action Committee (FRAC) code U8) and (46.4) “triazolopyrimidine fungicides”. The thiazole carboxamides include ethaboxam. The phenylacetamides include cyflufenamid and N-[[(cyclopropylmethoxy)amino][6-(difluoromethoxy)-2,3-difluorophenyl]-methylene]benzeneacetamide. The arylphenylketones include benzophenones such as metrafenone and benzoylpyridines such as pyriofenone. The triazolopyrimidines include ametoctradin. Class (46) (i.e. “Fungicides other than classes (1) through (45)”) also includes bethoxazin, fluxapyroxad, neo-asozin (ferric methanearsonate), pyrrolnitrin, quinomethionate, tebufloquin, isofetamid, N-[2-[4-[[3-(4-chlorophenyl)-2-propyn-1-yl]oxy]-3-methoxyphenyl]ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)-amino]butanamide, 2-[[2-fluoro-5-(trifluoromethyl)phenyl]thio]-2-[3-(2-methoxyphenyl)-2-thiazolidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl) [1,2,4]triazolo[1,5-a]pyrimidine, N-(4-chloro-2-nitrophenyl)-N-ethyl-4-methylbenzenesulfonamide, N-[[(cyclopropylmethoxy)amino][6-(difluoromethoxy)-2,3-difluorophenyl]methylene]benzeneacetamide, AP-[4-[4-chloro-3-(trifluoromethyl)phenoxy]-2,5-dimethylphenyl]-N-ethyl-N-methylmethanimidamide, 1-[(2-propenylthio)carbonyl]-2-(1-methylethyl)-4-(2-methylphenyl)-5-amino-1H-pyrazol-3-one, N′-[4-[[3-[(4-chlorophenyl)methyl]-1,2,4-thiadiazol-5-yl]oxy]-2,5-dimethylphenyl]-N-ethyl-N-methyl-methanimidamide, 1,1-dimethylethyl N-[6-[[[[(1-methyl-1H-tetrazol-5-yl)phenylmethylene]amino]oxy]methyl]-2-pyridinyl]carbamate, 3-butyn-1-yl N-[6-[[[[(1-methyl-1H-tetrazol-5-yl)phenylmethylene]amino]oxy]methyl]-2-pyridinyl]carbamate, 2,6-dimethyl-1H,5H-[1,4]dithiino[2,3-c:5,6-c′]dipyrrole-1,3,5,7(2H,6H)-tetrone, 5-fluoro-2-[(4-methylphenyl)-methoxy]-4-pyrimidinamine, 5-fluoro-2-[(4-fluorophenyl)methoxy]-4-pyrimidinamine, α-[3-(4-chloro-2-fluorophenyl)-5-(2,4-difluorophenyl)isoxazol-4-yl]pyrid-3-ylmethanol, (αS)-[3-(4-chloro-2-fluorophenyl)-5-(2,4-difluorophenyl)isoxazol-4-yl]pyrid-3-ylmethanol and (αR)-[3-(4-chloro-2-fluorophenyl)-5-(2,4-difluorophenyl)isoxazol-4-yl]pyrid-3-ylmethanol.

Therefore, of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, acrinathrin, amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifo s-methyl, chromafenozide, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, meperfluthrin, metaflumizone, metaldehyde, methamidophos, methidathion, methomyl, methoprene, methoxychlor, methoxyfenozide, metofluthrin, milbemycin oxime, monocrotophos, nicotine, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulfoxaflor, sulprofos, tebufenozide, teflubenzuron, tefluthrin, terbufos, tetrachlorvinphos, tetramethylfluthrin, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to a compound of Formula 1 include at least one compound selected from the group consisting of (1) alkylenebis(dithiocarbamate) fungicides; (2) cymoxanil; (3) phenylamide fungicides; (4) proquinazid (6-iodo-3-propyl-2-propyloxy-4(3H)-quinazolinone); (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin, pyrametostrobin, pyraoxystrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin.

The control efficacy of compounds of this invention on specific pathogens is demonstrated in TABLE A below. The pathogen control protection afforded by the compounds is not limited, however, to the species described in Tests A-G below.

Descriptions of the compounds are provided in INDEX TABLE A below. The following abbreviations are used in the index table: “Cmpd. No.” means compound number and “Ex.” stands for “Example” and is followed by a number indicating in which example the compound is prepared. In INDEX TABLE A, the numerical value reported in the column “AP⁺ (M+1)”, is the molecular weight of the observed molecular ion formed by addition of H⁺ (molecular weight of 1) to the molecule having the greatest isotopic abundance (i.e. M); the numerical value reported in the column “AP⁻ (M−1)”, is the molecular weight of the observed molecular ion formed by loss of H⁺ (molecular weight of 1) from the molecule having the greatest isotopic abundance (i.e. M). The presence of molecular ions containing one or more higher atomic weight isotopes of lower abundance (e.g., ³⁷Cl, ⁸¹Br) is not reported. The reported M+1 and M−1 peaks were observed by mass spectrometry using atmospheric pressure chemical ionization (AP+).

INDEX TABLE A

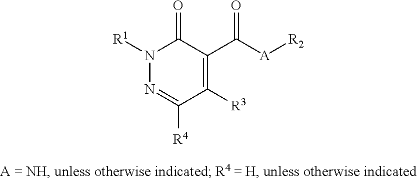

| Cmpd | | | | AP⁻ | AP⁺ |
|---|---|---|---|---|---|
| No. | R¹ (and A) | R² | R³ (and R⁴) | (M − 1) | (M + 1) |
| 1 | Me | 2,2-difluoro-1,3- | OH | * | |
| | | benzodioxol-5-yl | | | |
| 2 | Me | Ph(4-CF₃) | OH | 312.1 | |
| (Ex. 1) | | | | | |
| 3 | Me | Ph(4-OCF₃) | R³ = OH | * | |
| | | | R⁴ = Me | | |
| 4 | CH₂CH₂Me | Ph(4-OCF₃) | OH | * | |
| 5 | CH₂Me | Ph(4-OCF₃) | OH | * | |
| 6 | Me | pyridin-3-yl(6-Cl) | OH | * | |
| 7 | Me | pyridin-3-yl(6-Cl) | OMe | * | |
| 8 | Me | Ph(4-Cl) | OMe | 292.1 | |
| 9 | Me | Ph(4-Cl) | OH | * | |
| 10 | Me | Ph(2-Cl) | OH | * | |
| 11 | Me | Ph(3-Cl) | OH | * | |
| 12 | CH₂Me | pyridin-3-yl(6-Cl) | OH | * | |
| 13 | Me | 5-Me-1,3,4-thiadiazol-2-yl | OH | * | |
| 14 | Ph | pyridin-3-yl(6-Cl) | OH | * | |
| 15 | Me | Ph(3-CN) | OH | * | |
| 16 | Me | Ph(2-Me,4-Cl) | OH | * | |
| 17 | Me | Ph(3,4-di-Cl) | OH | * | |
| 18 | Me | Ph(3-CF₃,4-Cl) | OH | * | |
| 19 | Me | 8-quinolyl | OH | * | |
| 20 | Me | Ph(2,6-di-F) | OH | * | |
| 21 | Me | Ph(4-OCF₃) | OH | 328.3 | 330.3 |
| (Ex. 2) | | | | | |
| 22 | Me | Bn(4-Cl) | OH | * | |
| 23 | R¹ = Me | Ph(4-Cl) | OH | * | |
| | A = NMe | | | | |
| 24 | Me | Ph(2-CN,4-Cl) | OH | * | |
| 25 | Me | Ph(3-F) | OH | * | |
| 26 | Me | Ph(2-CF_{₃}) | R³ = OH | 388.2 | 390.2 |
| | | | R⁴ = Ph | | |
| 27 | Me | Ph-2-[Ph(3,4,5-tri-F)] | OH | 374.5 | 376.4 |
| 28 | Me | Ph-2-[Ph(4-Cl)] | OH | 354.5 | 356.5 |
| 29 | Me | pyrazol-2-yl(1-Me,4-CF₃) | OH | 316.5 | 318.4 |
| 30 | Me | Ph(4-OMe) | Cl | 292.5 | 294.4 |
| 31 | Me | Ph(4-Me) | Cl | 276.4 | 278.3 |
| 32 | Me | Ph-2-[Ph(2,3,4-tri-F)] | Cl | 392.5 | 394.4 |
| 33 | Me | Ph-2-[Ph(4-Cl)] | Cl | 372.5 | 374.4 |
| 34 | Me | Ph(2-OMe) | OH | 274.5 | 276.4 |
| 35 | Me | Ph(2-Br) | OH | 322.4 | 326.3 |
| 36 | Me | Ph(2-F) | OH | 262.4 | 264.3 |
| 37 | Me | Ph(2,4-di-Cl) | OH | | 312.3 |
| 38 | Me | pyrimidin-2-yl(5-Cl) | OH | 280.2 | 282.2 |
| 39 | Me | cyclohexane | OH | 250.3 | 252.2 |
| 40 | Me | Ph(4-F) | OH | 262.3 | |
| 41 | Me | Ph(4-OCF₃) | Cl | 346.4 | 348.3 |
| (Ex. 3) | | | | | |
| 42 | Me | Ph(4-OCF₃) | Br | 392.4 | 394.3 |
| (Ex. 4) | | | | | |
| 43 | Me | Ph(4-F) | Cl | 280.4 | 282.4 |
| 44 | Me | Ph-3-[Ph(4-Cl)] | OH | 354.5 | 356.4 |
| 45 | Me | pyridin-2-yl-(3-Cl) | OH | 279.2 | 281.2 |
| 46 | Me | Ph-4-[Ph(4-F)] | OH | 338.5 | 340.4 |
| 47 | Me | Ph(4-OCHF₂) | OH | 310.5 | 312.4 |
| 48 | Me | Ph(2-F,4-OCF₃) | OH | 346.4 | 348.3 |
| 49 | Me | Ph(4-S(═O)₂Me) | OH | 322.4 | 324.3 |
| 50 | Me | Ph(2F,4-CF₃) | OH | 330.4 | 332.3 |
| 51 | Me | Ph(2-F,4-OCF₃) | Cl | 364.1 | 366.1 |
| 52 | Me | Ph(2-F,4-CF₃) | Cl | 348.1 | 350.1 |
| 53 | Me | Ph(4-OCF₃) | OC(═O)C(Me)₃ | 412.2 | |
| 54 | Me | Ph(3-Me) | OH | 258.5 | 260.4 |
| 55 | Me | Ph(3-OMe) | OH | 274.5 | 276.4 |
| 56 | Me | Ph(3-CF₃) | OH | 312.5 | 314.4 |
| 57 | Me | Ph(3-OCF₃) | OH | 328.4 | 330.4 |
| 58 | Me | Ph(4-Br) | Cl | 374.5 | 376.4 |
| 59 | Me | Ph(4-Br) | OMe | 354.5 | 356.5 |
| 60 | Ph | Ph(4-OCF₃) | OH | | 392.2 |
| 61 | Me | pyridin-3-yl(6-CF₃) | OH | 292.5 | 294.4 |
| 62 | Me | Ph(4-CN) | OH | 276.4 | 278.3 |
| 63 | Me | Ph(3-OCF₃) | Cl | 392.5 | 394.4 |
| 64 | Me | Ph(3-CF3) | Cl | 372.5 | 374.4 |
| 65 | Me | Ph(2,4,6-tri-F) | OH | 274.5 | 276.4 |
| 66 | R¹ = Me | Ph(4-OCF₃) | OC(═O)CH₂C(Me)₃ | 322.4 | 326.3 |
| | A is | | | | |
| | NC(═O)CH₂C(Me)₃ | | | | |
| 67 | Me | Ph(4-SCF₃) | OH | 262.4 | 264.3 |
| 68 | Me | Ph(4-OCF₃) | R³ = OH | | 312.3 |
| | | | R⁴ = Cl | | |
| 69 | Me | Ph(4-OCF₃) | R³ = OH | 280.2 | 282.2 |
| | | | R⁴ = OMe | | |
| 70 | Me | Ph(4-CF₃) | R³ = OH | 250.3 | 252.2 |
| | | | R⁴ = OMe | | |
| 71 | Ph-(3-CF₃) | Ph(4-OCF₃) | OH | 262.3 | |
| 72 | Me | Ph(4-OCF₃) | H | 346.4 | 348.3 |
| 73 | Me | Ph(4-CF₃) | H | 392.4 | 394.3 |
| 74 | Me | Ph-2-[Ph(3,4,5-tri-F)] | H | 280.4 | 282.4 |
| 75 | Me | Ph(4-Br) | OH | 354.5 | 356.4 |
| 76 | Me | Ph(3,5-di-Br) | OH | 279.2 | 281.2 |
| 77 | Me | Ph(4-OCF₂CHCF₂) | OH | 338.5 | 340.4 |
| 78 | Me | CH(CH₃)CH₂-Ph(2,4-di-Cl) | OH | 310.5 | 312.4 |
| 79 | Me | Ph(4-OPh) | OH | 346.4 | 348.3 |
| 80 | Me | Ph(2-OPh) | OH | 322.4 | 324.3 |
| 81 | R¹ = Me | Ph(4-OCF₃) | OC(═O)—Ph | 330.4 | 332.3 |
| | A is NC(═O)—Ph | | | | |
| 82 | Me | Ph(2-OPh) | Cl | 364.1 | 366.1 |
| 83 | Me | Ph(4-OCF₃) | OC(═O)Me | 348.1 | 350.1 |
| 84 | R¹ = Me | Ph(4-OCF₃) | OC(═O)Me | | 414.0 |
| | A is NC(═O)Me | | | | |
| 85 | Me | Ph(4-OCF₃) | OC(═O)CH(Me)₂ | 258.5 | 260.4 |
| 86 | R¹ = Me | Ph(4-OCH₃) | OC(═O)CH(Me)₂ | 274.5 | 276.4 |
| | A is NC(═O)CH(Me)₂ | | | | |
| 87 | Me | Ph(4-OCF₃) | OC(═O)CH₂CH₂Me | * | |
| 88 | R¹ = Me | Ph(4-OCF₃) | OC(═O)Me | * | |
| | A is NC(═O)OMe | | | | |
| 89 | Me | Ph(4-OCF₃) | A and R³ are taken together | * | 356 |
| (Ex. 6) | (see R³ column for | | as NC(═O)O | | |
| | definition of A) | | | | |
| 90 | Me | Ph(4-OCF₃) | OS(═O)₂Me | * | |
| (Ex. 5) | | | | | |
| 91 | Me | Ph(4-SF₅) | OH | 370.3 | 372.3 |
| 92 | Me | Ph(4-OMe) | OH | 274.3 | 276.2 |
| 93 | Me | Ph(4-Me) | OH | 258.3 | 260.3 |
| 94 | Me | pyridin-3-yl(6-CF₃) | OS(═O)₂Me | | 393.3 |
| 95 | Me | pyrimidin-2-yl(5-Cl) | OS(═O)₂Me | * | |
| 96 | Me | Ph(4-CF₃) | OS(═O)₂Me | | 392.1 |
| 97 | Me | Ph(4-OCHF₂) | OS(═O)₂Me | | 390.1 |
| 98 | Me | Ph(4-CN) | OS(═O)₂Me | | 349.1 |
| 99 | Me | Ph(4-SF₅) | OS(═O)₂Me | | 450.1 |
| 100 | Me | Ph(2-F,4-OCF₃) | OS(═O)₂Me | | 426.1 |
| 101 | Me | Ph(4-F) | OS(═O)₂Me | | 342.1 |
| 102 | Me | Ph(2-F,4-CF₃₎ | OS(═O)₂Me | | 410.1 |
| 103 | Me | Ph-2-[Ph(3,4,5-tri-F)] | OS(═O)₂Me | | 454.2 |
| 104 | Me | Ph(2-F) | OS(═O)₂Me | | |
| 105 | Me | Ph(3-Me) | OS(═O)₂Me | | 338.2 |
| 106 | Me | Ph(4-OPh) | OS(═O)₂Me | | 416.2 |
| 107 | Me | Ph(4-OCF₃) | OS(═O)₂—Ph | 468 | 470.2 |
| 108 | Me | Ph(3-OMe) | OS(═O)₂Me | * | |
| 109 | Me | Ph(4-OCF₃) | SMe | | 360.1 |
| 110 | Me | Ph(4-OCF₃) | S(═O)Me | | 376.1 |
| 111 | Me | Ph(4-OCF₃) | S(═O)₂Me | | 392 |
| 112 | Me | Ph(4-OCF₃) | OS(═O)₂Ph(4-Me) | 482.1 | 484.1 |
| 113 | Me | Ph(4-SF₅) | O⁻Na⁺ | * | |
| 114 | Me | pyrazin-2-yl(5-Cl) | OH | 280.1 | 282.1 |
| 115 | CH₂Ph(4-OCH₃) | Ph(4-OCF₃) | OH | 434.3 | |
| 116 | Me | Ph(4-OCF₃) | OS(═O)₂CH₂Ph | | 484.3 |
| 117 | Me | Ph(2-Me,4-CF₃) | OH | 326.1 | 328.2 |
| 118 | Me | Ph(2,4,6-tri-Cl) | OH | 348.2 | |
| 119 | CH₂CF₃ | Ph(4-OCF₃) | OH | 396.4 | |
| 120 | cyclopropyl | Ph(4-OCF₃) | OH | | 356.2 |
| 121 | Me | Ph(4-C(═O)OCH₃) | OH | 302.3 | 304.3 |
| 122 | Me | Ph(2-CF₃) | OH | 312.3 | 314.2 |
| 123 | Me | Ph(2-F,4-Cl) | OH | 296.1 | |
| 124 | Me | Ph(2-Me,4-OCF₃) | OH | 342.3 | 344.3 |
| 125 | Me | pyrimidin-3-yl(2-Cl) | OH | 280.1 | 282.1 |
| 126 | Me | Ph(2-OCF₃) | OH | 328.3 | 330.3 |
| 127 | Me | Ph(4-OCF₃) | NHOMe | 357.3 | |
| 128 | Me | Ph(2-F,4-CF₃) | NMe₂ | 357.4 | 359.4 |
| 129 | Me | Ph(4-S(═O)₂CF₃) | OH | 376.2 | 378 |
| 130 | Me | Ph(2,3-F2,4-CF₃) | OH | 380.3 | 382.3 |
| 131 | (CH₃)₂CH | Ph(4-OCF₃) | OH | | 358.2 |
| 132 | Me | Ph(4-OCF₃) | E-OCH₂CH═CHPh | 444.5 | |
| 133 | Me | Ph(2,6-di-Cl,4-OCF₃) | OH | 396.3 | |
| 134 | Me | Ph(2-Cl,4-F) | OH | 296.2 | 298.2 |
| 135 | Me | Ph(4-OCF₃) | N(Me)(OMe) | 371.4 | |
| 136 | H | Ph(4-OCF₃) | OH | 314.3 | |
| 137 | Me | Ph(2-Cl,4-CF₃) | OH | 346.3 | 348 |
| 138 | Me | Ph(4-CF₂CF₃) | OH | 362.3 | 364.4 |
| 139 | R¹ = Me | Ph(4-OCF₃) | OC(═O)CH₂CH₂CH₃ | * | |
| | A is | | | | |
| | NC(═O)CH₂CH₂CH₃ | | | | |
| 140 | Me | Ph(4-CH₂CF₃) | OH | 326.3 | 328.3 |
| 141 | Me | Ph(2,6-di-Cl) | OH | 296.1 | |
| 142 | Me | Ph(2-Cl,4-OCF₃) | OH | 362.3 | 364.3 |
| 143 | Me | Ph(4-SF₅) | O⁻N(Bu)₄⁺ | * | |
| 144 | Me | Ph(2,4-di-F) | OH | 280.1 | 282.1 |
| 145 | Me | Ph(2-CF₃,4-F) | OH | 330.3 | |
| 146 | Me | Ph(4-OCF₃) | NHMe | 341.3 | |
| 147 | Me | Ph(2,4-(CF₃)₂) | OH | 380.3 | 382.3 |
| 148 | Me | Ph(2-CF₃,4-OCF₃) | OH | 396.2 | |
| 149 | Me | Ph(4-Br) | Cl | * | |
| 150 | Me | Ph(4-OCF₃) | OP(═O)(OEt)₂ | 464.4 | 466.4 |
| 151 | Me | Ph(3-phenoxy) | OH | 336.4 | |

*See INDEX TABLE B for ¹H NMR data
E indicates E geometry of double bond

INDEX TABLE B

| No. | ¹H NMR Data (CDCl₃ solution unless indicated otherwise)^{a} |
|---|---|
| 1 | δ 14.62 (s, 1H), 12.08 (br s, 1H), 7.79 (s, 1H), 7.72 (s, 1H), 7.16- |
| | 7.20 (m, 1H), 7.02-7.06 (m, 1H), 3.81 (s, 3H) |
| 3 | δ 15.02 (s, 1H), 12.25 (br s, 1H), 7.68-7.73 (m, 2H), 7.21-7.25 |
| | (m, 2H), 3.77 (s, 3H), 2.34 (s, 3H) |
| 4 | δ 14.73 (s, 1H), 12.14 (br s, 1H), 7.80 (s, 1H), 7.67-7.73 (m, 2H), |
| | 7.20-7.25 (m, 2H), 4.11-4.17 (m, 2H), 1.78-1.90 (m, 2H), 0.95- |
| | 1.02 (m, 3H) |
| 5 | δ 14.71 (s, 1H), 12.14 (br s, 1H), 7.81 (s, 1H), 7.68-7.73 (m, 2H), |
| | 7.21-7.25 (m, 2H), 4.20-4.27 (m, 2H), 1.37-1.43 (m, 3H) |
| 6 | δ 14.34 (s, 1H), 12.20 (br s, 1H), 8.60-8.63 (m, 1H), 8.14-8.18 |
| | (m, 1H), 7.81 (s, 1H), 7.33-7.37 (m, 1H), 3.82 (s, 3H) |
| 7 | δ 11.91 (br s, 1H), 8.57-8.62 (m, 1H), 8.24-8.30 (m, 1H), 8.02 |
| | (s, 1H), 7.27-7.32 (m, 1H), 4.18 (s, 3H), 3.88 (s, 3H) |
| 9 | δ 14.74 (s, 1H), 12.02 (br s, 1H), 7.78 (s, 1H), 7.60-7.64 (m, 2H), |
| | 7.32-7.37 (m, 2H), 3.80 (s, 3H) |
| 10 | δ 14.71 (s, 1H), 12.34 (br s, 1H), 8.38-8.42 (m, 1H), 7.77-7.79 |
| | (m, 1H), 7.44-7.48 (m, 1H), 7.28-7.34 (m, 1H), 7.10-7.17 (m, 1H), |
| | 3.84 (s, 3H) |
| 11 | δ 14.67 (s, 1H), 12.06 (br s, 1H), 7.81-7.85 (m, 1H), 7.78 (s, 1H), |
| | 7.45-7.50 (m, 1H), 7.27-7.34 (m, 1H), 7.14-7.19 (m, 1H), 3.81 |
| | (s, 3H) |
| 12 | δ 14.36 (s, 1H), 12.28 (br s, 1H), 8.62 (d, J = 1.0 Hz, 1H), 8.14- |
| | 8.19 (m, 1H), 7.83 (s, 1H), 7.35 (d, J = 1.0 Hz, 1H), 4.21-4.28 |
| | (m, 2H), 1.38-1.43 (m, 3H) |
| 13 | δ 7.98 (s, 1H), 3.91 (s, 3H), 2.74 (s, 3H) |
| 14 | δ 14.70 (s, 1H), 12.12 (s, 1H), 8.59 (s, 1H), 8.06-8.24 (m, 1H), |
| | 7.98 (s, 1H), 7.44-7.59 (m, 5H), 7.34 (d, J = 8.4 Hz, 2H) |
| 15 | δ 14.40-14.44 (m, 1H), 12.20-12.26 (m, 1H), 8.16 (s, 1H), 7.75- |
| | 7.82 (m, 2H), 7.45-7.49 (m, 2H), 3.81 (s, 3H) |
| 16 | δ 14.84 (s, 1H), 11.87 (s, 1H), 8.05-8.11 (m, 1H), 7.77-7.80 |
| | (m, 1H), 7.25-7.27 (m, 1H), 7.19-7.21 (m, 1H), 3.82 (s, 3H), |
| | 2.40 (s, 3H) |
| 17 | δ 14.49 (s, 1H), 12.12 (br s, 1H), 7.95 (s, 1H), 7.78 (s, 1H), 7.36- |
| | 7.52 (m, 2H), 3.80 (s, 3H) |
| 18 | δ 14.41 (s, 1H), 12.24 (br s, 1H), 8.04 (d, J = 1.0 Hz, 1H), 7.79- |
| | 7.85 (m, 2H), 7.50 (d, J = 1.0 Hz, 1H), 3.82 (s, 3H) |
| 19 | δ 15.13 (s, 1H), 13.59 (br s, 1H), 9.05-9.08 (m, 1H), 8.84-8.87 |
| | (m, 1H), 8.18-8.22 (m, 1H), 7.79 (s, 1H), 7.55-7.66 (m, 2H), 7.48- |
| | 7.53 (m, 1H), 3.90 (s, 3H) |
| 20 | δ 14.33 (s, 1H), 11.43 (br s, 1H), 7.79 (s, 1H), 7.27-7.33 (m, 1H), |
| | 6.98-7.05 (m, 2H), 3.82 (s, 3H) |
| 22 | δ 15.04 (s, 1H), 9.93 (br s, 1H), 7.67 (s, 1H), 7.33-7.38 (m, 2H), |
| | 7.21-7.25 (m, 2H), 4.58-4.62 (m, 2H), 3.73-3.76 (m, 3H) |
| 23 | δ 7.62-7.64 (m, 1H), 7.26-7.30 (m, 2H), 7.07-7.12 (m, 2H), 3.42- |
| | 3.46 (m, 6H) |
| 24 | δ 14.08 (s, 1H), 12.81 (s, 1H), 8.37-8.43 (m, 1H), 7.80 (s, 1H), |
| | 7.62-7.65 (m, 1H), 7.56-7.61 (m, 1H), 3.84 (s, 3H) |
| 25 | δ 14.69 (s, 1H), 12.07 (br s, 1H), 7.78 (s, 1H), 7.59-7.65 (m, 1H), |
| | 7.28-7.36 (m, 2H), 6.86-6.92 (m, 1H), 3.81 (s, 3H) |
| 87 | δ 11.71 (brs, 1H), 7.77 (s, 1H), 7.67-7.69 (m, 2H), 7.18-7.20 (m, |
| | 2H), 3.90 (s, 3H), 2.71 (t, 2H), 1.78-1.85 (m, 2H), 1.07 (t, 3H) |
| 88 | δ 7.91 (s, 1H), 7.34-7.36 (m, 2H), 7.28-7.30 (m, 2H), 3.94 (s, 3H), |
| | 3.82 (s, 3H), 3.70 (s, 3H) |
| 89 | δ 7.97 (s, 1H), 7.37 (m, 2H), 7.31 (m, 2H), 3.88 (s, 3H) |
| 90 | δ 11.54 (brs, 1H), 8.08 (s, 3H), 7.70-7.72 (m, 2H), 7.21-7.23 |
| | (m, 2H), 3.92 (s, 3H), 3.55 (s, 3H) |
| 95 | δ 12.17 (brs, 1H), 8.62 (s, 2H), 8.13 (s, 1H), 3.92 (s, 3H), 3.55 |
| | (s, 3H) |
| 108 | δ 11.37 (brs, 1H), 8.07 (s, 1H), 7.36-7.37 (m, 1H), 7.24-7.28 |
| | (m, 1H), 7.19-7.21 (m, 1H), 6.71-6.73 (m, 1H), 3.91 (s, 3H), 3.83 |
| | (s, 3H), 3.55 (s, 3H) |
| 113 | (DMSO-d₆) δ 13.66 (s, 1H), 7.80 (m, 2H), 7.75 (m, 2H), 7.20 |
| | (s, 1H), 3.45 (s, 3H) |
| 139 | δ 7.78 (s, 1H), 7.37 (m, 2H), 7.31 (m, 2H), 2.98 (s, 3H), 2.54 |
| | (m, 2H), 2.30 (m, 2H), 1.74 (m, 2H), 1.60 (m, 2H), 1.01 (m, 3H), |
| | 0.87 (m, 3H) |
| 143 | δ 13.37 (brs, 1H), 7.81 (m, 2H), 7.59 (m, 2H), 7.37 (s, 1H), 3.57 |
| | (s, 3H), 3.22 (m, 8H), 1.56 (m, 8H), 1.36 (m, 8H), 0.95 (m, 12H) |
| 149 | 7.96 (s, 1H), 7.67 (m, 2H), 7.14 (m, 2H), 3.87 (s, 3H) |

^{a1}H NMR data are in ppm downfield from tetramethylsilane at 500 MHz. Couplings are designated by (s)-singlet, (d)-doublet, (t)-triplet, (m)-multiplet and (dd)-doublet of doublets.

### Biological Examples of the Invention

General protocol for preparing test solutions for Tests AG: the test compounds were first dissolved in acetone in an amount equal to 3% of the final volume and then suspended at the desired concentration (in ppm) in acetone and purified water (50/50 mix by volume) containing the surfactant PEG400 (polyhydric alcohol esters). The resulting test suspensions were then used in Tests AG. Compounds were sprayed at a concentration of 250 ppm or other indicated rate to the point of run-off on the test plants.

### TEST A

The test solution was sprayed to the point of run-off on soybean seedlings. The following day the seedlings were inoculated with a spore suspension of Phakopsora pachyrhizi (the causal agent of Asian soybean rust) and incubated in a saturated atmosphere at 22° C. for 24 h and incubated in a growth chamber at 22° C. for 8 days, after which time visual disease ratings were made.

### TEST B

The test solution was sprayed to the point of run-off on grape seedlings. The following day the seedlings were inoculated with a spore suspension of Plasmopara viticola (the causal agent of grape downy mildew) and incubated in a saturated atmosphere at 20° C. for 24 h, moved to a growth chamber at 20° C. for 6 days, and then incubated in a saturated atmosphere at 20° C. for 24 h, after which time disease ratings were made.

### TEST C

The test solution was sprayed to the point of run-off on tomato seedlings. The following day the seedlings were inoculated with a spore suspension of Botrytis cinerea (the causal agent of gray mold on many crops) and incubated in a saturated atmosphere at 20° C. for 48 h, and moved to a growth chamber at 27° C. for 3 days, after which time visual disease ratings were made.

### TEST D

The test solution was sprayed to the point of run-off on tomato seedlings. The following day the seedlings were inoculated with a spore suspension of Phytophthora infestans (the causal agent of tomato late blight) and incubated in a saturated atmosphere at 20° C. for 24 h, and then moved to a growth chamber at 20° C. for 5 days, after which time disease ratings were visually made.

### TEST E

The test solution was sprayed to the point of run-off on wheat seedlings. The following day the seedlings were inoculated with a spore suspension of Zymoseptoria tritici (the causal agent of wheat leaf blotch) and incubated in a saturated atmosphere at 24° C. for 48 h, and then moved to a growth chamber at 20° C. for 17 days, after which time visual disease ratings were made.

### TEST F

The test solution was sprayed to the point of run-off on wheat seedlings. The following day the seedlings were inoculated with a spore suspension of Puccinia recondita f. sp. tritici; (the causal agent of wheat leaf rust) and incubated in a saturated atmosphere at 20° C. for 24 h, and then moved to a growth chamber at 20° C. for 6 days, after which time visual disease ratings were made.

### TEST G

The test solution was sprayed to the point of run-off on wheat seedlings. The following day the seedlings were inoculated with a spore dust of Erysiphe graminis f. sp. tritici, (the causal agent of wheat powdery mildew) and incubated in a growth chamber at 20° C. for 8 days, after which time visual disease ratings were made.

Results for Tests AG are given in TABLE A. In the Table, a rating of 100 indicates 100% disease control and a rating of 0 indicates no disease control (relative to the controls). A dash (-) indicates no test results. Compounds in test solutions were applied at 250 ppm, unless otherwise indicated in the table.

TABLE A

| Cmpd | Rate | Test | Test | Test | Test | Test | Test | Test |
|---|---|---|---|---|---|---|---|---|
| No. | (ppm) | A | B | C | D | E | F | G |
| 1 | 200 | — | — | 0 | 0 | | 98 | 40 |
| 2 | 250 | — | 97 | 0 | 99 | 97 | 100 | 97 |
| 3 | 200 | — | — | 0 | 0 | 0 | 0 | 37 |
| 4 | 200 | — | — | 0 | 99 | 0 | 95 | 97 |
| 5 | 200 | — | — | 10 | 50 | 0 | 84 | 32 |
| 6 | 250 | | 85 | 99 | 88 | 57 | 99 | 0 |
| 7 | — | — | — | — | — | — | — | — |
| 8 | 200 | — | — | 0 | 0 | — | 0 | 0 |
| 9 | 200 | — | — | 0 | 0 | — | 99 | 96 |
| 10 | 200 | — | — | 99 | 0 | — | 97 | 0 |
| 11 | 200 | — | — | 48 | 0 | — | 95 | 31 |
| 12 | 200 | — | — | — | 0 | — | 91 | 93 |
| 13 | 250 | — | 65 | 0 | 0 | 0 | 0 | 0 |
| 14 | 200 | — | — | 90 | 0 | — | 98 | 0 |
| 15 | 200 | — | — | 16 | 79 | — | 91 | 0 |
| 16 | 200 | — | — | 0 | 91 | — | 99 | 45 |
| 17 | 200 | — | — | 0 | 57 | — | 99 | 0 |
| 18 | 200 | — | — | 0 | 0 | — | 83 | 46 |
| 19 | 200 | — | — | 0 | 0 | — | 96 | 0 |
| 20 | 200 | — | — | 0 | 0 | — | 0 | 0 |
| 21 | 250 | | 93 | 0 | 97 | 98 | 99 | 98 |
| 22 | 40 | — | — | 0 | 0 | — | 41 | 0 |
| 23 | 40 | — | — | 0 | 0 | — | 0 | 0 |
| 24 | 200 | — | — | 99 | 0 | — | 91 | 0 |
| 25 | 200 | — | — | 0 | 0* | — | 89 | 0 |
| 26 | 250 | — | 49 | 0 | 58 | 0 | 68 | 84 |
| 27 | 250 | 0 | — | 0 | 0 | 0 | 98 | 0 |
| 28 | 250 | 0 | — | 0 | 0 | 0 | 97 | 27 |
| 29 | 250 | 0 | — | 0 | 0 | 0 | 68 | 27 |
| 30 | 250 | 60 | — | 0 | 0 | 0 | 96 | 0 |
| 31 | 250 | 65 | — | 92 | 0 | 0 | 95 | 0 |
| 32 | 250 | 94 | — | 0 | 0 | 0 | 96 | 0 |
| 33 | 250 | 48 | — | 0 | 0 | 0 | 97 | 0 |
| 34 | 250 | 0 | — | 0 | 0 | 0 | 80 | 0 |
| 35 | 250 | 0 | — | 0 | 71 | 0 | 98 | 0 |
| 36 | 250 | 31 | — | 0 | 94 | 0 | 98 | 78 |
| 37 | 250 | — | 86 | 0 | 95 | 51 | 94 | 76 |
| 38 | 250 | — | 20 | 0 | 0 | 0 | 68 | 0 |
| 39 | 250 | — | 70 | 0 | 0 | 0 | 0 | 69 |
| 40 | 250 | 56 | — | 96 | 73 | 96 | 95 | 92 |
| 41 | 250 | 87 | — | 0 | 0 | 97 | 89 | 0 |
| 42 | 250 | 79 | — | 0 | 0 | 99 | 86 | 0 |
| 43 | 250 | 78 | — | 79 | 68 | 4 | 97 | 0 |
| 44 | 250 | 0 | — | 0 | 0 | 0 | 86 | 0 |
| 45 | 250 | — | 88 | 99 | 94 | 0 | 98 | 0 |
| 46 | 250 | 77 | — | 16 | 40 | 0 | 80 | 0 |
| 47 | 250 | 38 | — | 0 | 88 | 77 | 99 | 0 |
| 48 | 250 | — | — | 62 | 97 | 96 | 99 | 97 |
| 49 | 250 | 0 | — | 0 | 0 | 0 | 68 | 0 |
| 50 | 250 | 0 | — | 85 | 99 | 98 | 100 | 98 |
| 51 | 125 | 99 | — | 97 | 57 | 89 | 96 | 0 |
| 52 | 160 | 97 | — | 59 | 90 | 99 | 99 | 39 |
| 53 | 250 | 79 | — | 39 | 99 | 79 | 100 | 98 |
| 54 | 250 | 0 | — | 0 | 0 | 0 | 95 | 0 |
| 55 | 250 | 0 | — | 0 | 0 | 0 | 89 | 0 |
| 56 | 250 | 0 | — | 0 | 61 | 0 | 92 | 35 |
| 57 | 250 | 0 | — | 0 | 0 | 8 | 84 | 98 |
| 58 | 250 | 92 | — | 0 | 68 | 0 | 99 | 0 |
| 59 | 250 | 0 | — | 0 | 0 | 0 | 0 | 0 |
| 60 | 250 | 0 | — | 0 | 88 | 92 | 88 | 57 |
| 61 | 250 | 0 | — | 0 | 0 | 75 | 100 | 61 |
| 62 | 250 | 0 | — | 0 | 57 | 0 | 74 | 0 |
| 63 | 270 | 59 | — | 53 | 53 | 98 | 86 | 48 |
| 64 | 265 | 92 | — | 76 | 53 | 94 | 91 | 0 |
| 65 | 250 | 79 | — | 0 | 0 | 18 | 28 | 87 |
| 66 | 250 | 58 | — | 0 | 58 | 95 | 100 | 96 |
| 67 | 250 | 12 | — | 0 | 66 | 89 | 97 | 56 |
| 68 | 250 | — | 86 | 17 | 50 | 0 | 68 | 0 |
| 69 | 250 | — | 96 | 0 | 94 | 53 | 68 | 0 |
| 70 | 250 | — | 74 | 0 | 88 | 0 | 28 | 0 |
| 71 | 250 | 12 | — | 0 | 0 | 0 | 0 | 0 |
| 72 | 250 | 0 | — | 0 | 0 | 0 | 0 | 0 |
| 73 | 250 | 0 | — | 0 | 0 | 0 | 0 | 0 |
| 74 | 250 | 0 | — | 0 | 0 | 0 | 0 | 0 |
| 75 | 250 | — | 86 | 0 | 93 | 83 | 98 | 13 |
| 76 | 250 | — | 24 | 0 | 53 | 0 | 86 | 0 |
| 77 | 250 | — | 76 | 0 | 91 | 4 | 86 | 0 |
| 78 | 250 | 60 | — | 0 | 53 | 86 | 98 | 99 |
| 79 | 250 | 13 | — | 0 | 0 | 0 | 74 | 0 |
| 80 | 250 | 0 | — | 0 | 0 | 0 | 79 | 0 |
| 81 | 250 | 0 | — | 0 | 0 | 67 | 98 | 0 |
| 82 | 250 | 25 | — | 0 | 0 | 30 | 88 | 0 |
| 83 | 250 | — | 98 | 15 | 98 | 100 | 100 | 99 |
| 84 | 280 | — | 99 | 0 | 100 | 100 | 100 | 99 |
| 85 | 250 | — | 95 | 9 | | 70 | 100 | 98 |
| 86 | 250 | — | 95 | 33 | 95 | 64 | 100 | 98 |
| 87 | 250 | — | 100 | 0 | | 100 | 100 | 99 |
| 88 | 50 | — | 91 | 0 | 57 | 32 | 83 | 89 |
| 89 | 250 | — | 99 | 0 | 67 | 97 | 99 | 61 |
| 90 | 250 | 98 | — | 60 | 78 | 100 | 98 | 0 |
| 91 | 250 | 46 | — | 0 | 97 | 100 | 99 | 89 |
| 92 | 250 | — | 92 | 99 | 68 | 59 | 89 | 35 |
| 93 | 250 | — | 94 | 15 | 93 | 65 | 91 | 87 |
| 94 | 250 | 77 | — | 0 | 0 | — | 99 | 52 |
| 95 | 250 | 73 | — | 0 | 0 | — | 68 | 0 |
| 96 | 250 | 98 | — | 30 | 0 | 97 | 99 | 84 |
| 97 | 250 | 95 | — | 40 | 95 | 100 | 99 | 91 |
| 98 | 250 | 99 | — | 98 | 9 | 95 | 97 | 0 |
| 99 | 250 | 99 | — | 50 | 92 | 100 | 98 | 83 |
| 100 | 250 | 94 | — | 98 | 0 | 92 | 95 | 0 |
| 101 | 250 | 78 | — | 0 | 9 | 78 | 99 | 0 |
| 102 | 250 | 99 | — | 41 | 26 | 95 | 99 | 0 |
| 103 | 250 | 84 | — | 0 | 0 | 96 | 90 | 73 |
| 104 | 250 | 87 | — | 0 | 0 | 94 | 89 | 65 |
| 105 | 250 | 86 | — | 0 | 33 | 86 | 86 | 0 |
| 106 | 250 | 98 | — | 0 | 85 | 83 | 86 | 81 |
| 107 | 250 | — | — | 0 | 96 | 71 | 100 | 98 |
| 108 | 250 | 0 | — | 0 | 0 | 0 | 68 | 0 |
| 109 | 250 | 0 | 0 | 0 | 0 | 0 | 0 | 55 |
| 110 | 50 | 92 | 0 | 0 | 0 | 88 | 74 | 0 |
| 111 | 250 | 98 | 0 | 0 | 9 | 93 | 96 | 0 |
| 112 | 250 | 25 | — | 0 | 46 | 43 | 95 | 0 |
| 113 | 250 | 73 | 96 | 0 | 81 | 79 | 94 | 90 |
| 114 | 250 | 39 | 90 | 0 | 80 | 55 | 95 | 0 |
| 115 | 250 | 0 | 0 | 0 | 0 | 26 | 68 | 0 |
| 116 | 250 | 25 | 99 | 0 | 0 | 60 | 97 | 0 |
| 117 | 250 | 0 | 100 | 0 | 97 | — | 99 | 90 |
| 118 | 250 | — | 71 | 0 | 100 | — | 86 | 97 |
| 119 | 250 | — | 100 | 0 | 87 | 73 | 97 | 99 |
| 120 | 250 | 13 | 98 | 0 | 97 | — | 96 | 89 |
| 121 | 250 | 0 | 86 | 0 | 99 | 14 | 80 | 0 |
| 122 | 250 | 0 | 98 | 0 | 95 | 96 | 86 | 96 |
| 123 | 250 | 81 | 90 | 63 | 90 | 91 | 100 | 92 |
| 124 | 250 | — | 99 | 0 | 99 | — | 99 | 99 |
| 125 | 250 | 81 | 83 | 0 | 76 | 11 | 86 | 0 |
| 126 | 250 | | 95 | 0 | | 82 | 89 | 93 |
| 127 | 250 | 0 | 23 | 0 | 0 | 27 | 0 | 0 |
| 128 | 250 | 79 | 100 | 0 | 67 | — | 68 | 0 |
| 129 | 250 | 0 | 98 | 0 | 82 | — | 89 | 81 |
| 130 | 250 | 77 | 98 | 0 | — | 99 | 97 | 99 |
| 131 | 250 | — | 98 | 0 | 99 | 51 | 91 | 100 |
| 132 | 250 | 0 | 25 | 0 | 17 | 50 | 91 | 97 |
| 133 | 250 | — | 100 | 0 | 89 | 100 | 86 | 100 |
| 134 | 250 | 75 | 92 | 0 | 0 | 45 | 32 | 90 |
| 135 | 250 | 0 | 27 | 0 | 0 | 2 | 0 | 0 |
| 136 | 250 | 0 | 100 | 0 | 91 | — | 93 | 0 |
| 137 | 250 | 0 | 100 | 0 | 97 | 30 | 96 | 47 |
| 138 | 250 | — | 100 | 0 | 99 | 99 | 100 | 99 |
| 139 | 250 | — | 100 | 0 | 0 | — | 100 | 100 |
| 140 | 250 | 0 | 100 | 0 | 96 | 88 | 99 | 27 |
| 141 | 250 | 50 | 96 | 0 | — | — | 68 | 64 |
| 142 | 250 | 71 | 99 | 26 | 92 | 77 | 99 | 98 |
| 143 | 250 | 62 | 100 | 0 | 70 | 88 | 94 | 69 |
| 144 | 250 | 0 | 100 | 0 | 99 | — | 99 | 87 |
| 145 | 250 | — | 100 | 0 | — | 99 | 99 | 99 |
| 146 | 250 | 0 | 10 | 0 | 0 | 0 | 0 | 0 |
| 147 | 250 | 0 | 97 | 0 | 99 | 87 | 99 | 98 |
| 148 | 250 | — | 79 | 0 | — | 79 | 86 | 98 |
| 149 | 250 | 92 | — | 0 | 68 | 0 | 99 | 0 |
| 150 | 250 | — | 100 | 0 | 0 | 96 | 100 | 99 |
| 151 | 250 | 0 | 83 | 0 | 0 | — | 85 | 13 |

What is claimed is:

1. A compound selected from Formula 1, N-oxides and salts thereof, 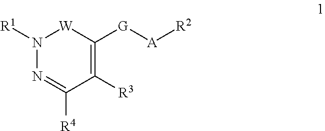
wherein
W is C(═O) or C(═S);
R¹ is H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₄-C₇ cycloalkylalkyl, C₄-C₇ alkylcycloalkyl, C₂-C₆ cyanoalkyl, C₁-C₆ alkoxy, C₁-C₆ haloalkoxy, C₂-C₆ alkoxyalkyl, C₂-C₆ haloalkoxyalkyl, C₃-C₆ alkoxyalkoxyalkyl, C₁-C₆ alkylthio, C₁-C₆ haloalkylthio, C₂-C₄ alkylthioalkyl, C₁-C₆ alkylsulfinyl, C₁-C₆ haloalkylsulfinyl, C₂-C₆ alkylsulfinylalkyl, C₁-C₆ alkylsulfonyl, C₁-C₆ haloalkylsulfonyl, C₂-C₆ alkylsulfonylalkyl, C₁-C₆ alkylsulfonyloxy, C₁-C₆ alkylaminosulfinyl, C₁-C₆ haloalkylaminosulfinyl, C₂-C₆ dialkylaminosulfinyl, C₁-C₆ alkylaminosulfonyl, C₁-C₆ haloalkylaminosulfonyl, C₂-C₆ dialkylaminosulfonyl, C₂-C₆ alkylcarbonyl, C₂-C₆ haloalkylcarbonyl, C₂-C₆ alkoxycarbonyl, C₃-C₆ alkylcarbonylalkyl, C₃-C₆ alkoxycarbonylalkyl, C₂-C₆ alkylaminocarbonyl, C₂-C₆ haloalkylaminocarbonyl, C₃-C₆ dialkylaminocarbonyl or -L(CR^{5a}R^{5b})ₘQ¹;
L is O or a direct bond;
each R^{5a} is independently H, cyano, halogen or C₁-C₄ alkyl;
each R^{5b} is independently H or C₁-C₄ alkyl;
Q¹ is a phenyl ring optionally substituted with up to 5 substituents independently selected from R^{6a}; or a 5- to 6-membered heterocyclic ring, each ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 5 substituents independently selected from R^{6a} on carbon atom ring members and R^{6b} on nitrogen atom ring members;
each R^{6a} is independently halogen, cyano, hydroxy, nitro, amino, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₄-C₆ alkylcycloalkyl, C₄-C₆ cycloalkylalkyl, C₂-C₆ cyanoalkyl, C₁-C₆ alkoxy, C₁-C₆ haloalkoxy, C₃-C₆ cycloalkoxy, C₂-C₆ alkylcarbonyloxy, C₂-C₆ alkylcarbonyl, C₂-C₆ haloalkylcarbonyl, C₃-C₆ alkylcarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylsulfonyloxy, C₁-C₆ haloalkylsulfonyloxy, C₁-C₆ alkylamino, C₂-C₆ dialkylamino, C₂-C₆ alkylcarbonylamino, CH(═O), —NHCH(═O), —SF₅, —SC≡N or —U—V-T;
G is C(═O), C(═S), S(═O), S(═O)₂; or C(═N)-J;
J is NR^{A}R^{B} or —ORS;
R^{A} is H, cyano, CHO, C₂-C₄ alkylcarbonyl, C₁-C₄ alkyl; or phenyl substituted or unsubstituted with halogen or C₁-C₄ alkyl;
R^{B} is H, cyano, CHO, C₁-C₆ alkyl, C₁-C₆ haloalkyl, C₂-C₆ alkenyl, C₃-C₆ alkynyl, C₂-C₈ alkoxyalkyl, C₂-C₈ haloalkoxyalkyl, C₂-C₈ alkylthioalkyl, C₂-C₈ alkylsulfinylalkyl, C₂-C₈ alkylsulfonylalkyl, C₂-C₈ alkylcarbonyl, C₂-C₈ haloalkylcarbonyl, C₄-C₁₀ cycloalkylcarbonyl, C₂-C₈ alkoxycarbonyl, C₂-C₈ haloalkoxycarbonyl, C₄-C₁₀ cycloalkoxycarbonyl, C₂-C₈ alkylaminocarbonyl, C₃-C₁₀ dialkylaminocarbonyl, C₄-C₁₀ cycloalkylaminocarbonyl, C₁-C₆ alkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₃-C₈ cycloalkylsulfinyl, C₁-C₆ alkylsulfonyl, C₁-C₆ haloalkylsulfonyl, C₃-C₈ cycloalkylsulfonyl, C₁-C₆ alkylaminosulfonyl, C₂-C₈ dialkylaminosulfonyl or C₃-C₁₀ trialkylsilyl; or a phenyl ring or a naphthalenyl ring system, each ring or ring system substituted or unsubstituted with up to 5 substituents independently selected from R¹⁶; or a 4- to 7-membered heterocyclic ring, substituted or unsubstituted on ring members with up to 5 substituents independently selected from R¹⁶; or
R^{A} and R^{B} are taken together along with the nitrogen atom to which they are both bonded to form a 4-, 5- or 6-membered ring containing ring members selected from carbon, oxygen, nitrogen and C(═O); or taken together as a 6- to 10-membered bicyclic ring system; or taken together as an 8- to 13-membered tricyclic ring system, each ring or ring system containing ring members selected from carbon, nitrogen and C(═O) and substituted or unsubstituted with halogen, cyano or C₁-C₄ alkyl;
R^{C} is H, CHO, C₁-C₆ alkyl, C₁-C₆ haloalkyl, C₂-C₆ alkenyl, C₃-C₆ alkynyl, C₂-C₈ alkoxyalkyl, C₂-C₈ haloalkoxyalkyl, C₂-C₈ alkylthioalkyl, C₂-C₈ alkylsulfinylalkyl, C₂-C₈ alkylsulfonylalkyl, C₂-C₈ alkylcarbonyl, C₂-C₈ haloalkylcarbonyl, C₄-C₁₀ cycloalkylcarbonyl, C₂-C₈ alkoxycarbonyl, C₂-C₈ haloalkoxycarbonyl, C₄-C₁₀ cycloalkoxycarbonyl, C₂-C₈ alkylaminocarbonyl, C₃-C₁₀ dialkylaminocarbonyl, C₄-C₁₀ cycloalkylaminocarbonyl, C₁-C₆ alkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₃-C₈ cycloalkylsulfinyl, C₁-C₆ alkylsulfonyl, C₁-C₆ haloalkylsulfonyl, C₃-C₈ cycloalkylsulfonyl, C₁-C₆ alkylaminosulfonyl, C₂-C₈ dialkylaminosulfonyl or C₃-C₁₀ trialkylsilyl; or a phenyl ring or a naphthalenyl ring system, each ring or ring system substituted or unsubstituted with up to 5 substituents independently selected from R¹⁶; or a 4- to 7-membered heterocyclic ring, substituted or unsubstituted on ring members with up to 5 substituents independently selected from R¹⁶;
A is N(R^{7a}), N(R^{7b})S(═O)ₙ or S(═O)ₙN(R^{7b});
R² is —(CR^{31a}R^{31b})ᵣQ²;
each R^{31a} is independently H, cyano, halogen or C₁-C₄ alkyl;
each R^{31b} is independently H or C₁-C₄ alkyl;
Q² is a phenyl ring or a naphthalenyl ring system, each ring or ring system optionally substituted with up to 5 substituents independently selected from R^{8a}; or a 5- to 6-membered heterocyclic ring, each ring containing ring members selected from carbon atoms and 1 to 4 heteroatoms independently selected from up to 2 O, up to 2 S and up to 4 N atoms, wherein up to 2 carbon ring members are independently selected from C(═O), C(═S), S(═O) and S(═O)₂, each ring optionally substituted with up to 5 substituents independently selected from R^{8a} on carbon atom ring members and R^{8b} on nitrogen atom ring members; or an 8- to 10-membered bicyclic ring system, each ring or ring system containing ring members selected from carbon atoms and 1 to 5 heteroatoms independently selected from up to 2 O, up to 2 S and up to 5 N atoms, wherein up to 3 carbon ring members are independently selected from C(═O) and C(═S), and the sulfur atom ring members are independently selected from S(═O)ₐ(═NR^{8c})_{b}, each ring or ring system optionally substituted with up to 5 substituents independently selected from R^{8a} on carbon atom ring members and selected from R^{8b} on nitrogen atom ring members;
R³ is H, halogen, OR⁹, OC(═Z)R¹⁰, SH, SR¹¹, S(═O)ᵤR¹¹, OS(═O)ᵤR¹¹, NR³²R³³, OC(═Z)NR¹²R¹³, S(═O)ᵤNR¹²R¹³, OP(═O)(R³⁴)₂, C₁-C₄ alkyl, C₁-C₄ haloalkyl, C₂-C₄ alkenyl, C₂-C₄ haloalkenyl, C₁-C₄ alkoxy, C₂-C₄ alkoxyalkyl, C₃-C₆ cycloalkyl or C₄-C₇ cycloalkylalkyl;
R⁴ is H, halogen, cyano, hydroxy, nitro, amino, C₁-C₃ alkyl, C₁-C₃ haloalkyl, C₂-C₄ alkenyl, C₂-C₄ haloalkenyl, C₂-C₄ alkynyl, C₂-C₄ haloalkynyl, C₁-C₃ alkoxy, C₁-C₃ haloalkoxy, C₂-C₄ alkoxyalkyl, C₁-C₃ alkylthio, C₁-C₃ haloalkylthio, C₁-C₃ alkylsulfinyl, C₁-C₃ haloalkylsulfinyl, C₂-C₄ alkylsulfinylalkyl, C₁-C₃ alkylsulfonyl, C₁-C₃ haloalkylsulfonyl, C₂-C₄ alkylsulfonylalkyl, C₂-C₄ alkylcarbonyl, C₂-C₄ haloalkylcarbonyl, C₂-C₄ alkoxycarbonyl, C₃-C₄ alkoxycarbonylalkyl, C₁-C₆ alkylamino, C₂-C₆ dialkylamino, C₂-C₄ alkylaminocarbonyl or C₃-C₄ dialkylaminocarbonyl; or a phenyl ring optionally substituted with up to 5 substituents independently selected from R²⁶;
each R^{8a} is independently halogen, cyano, hydroxy, nitro,
amino, —CH(═O), —C(═O)OH, —C(═O)NH₂, —C(R^{11a})═N—O—R^{12a}, —C(R^{11a})═N_R^{12a}, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₄-C₆ alkylcycloalkyl, C₄-C₆ cycloalkylalkyl, C₂-C₆ cyanoalkyl, C₁-C₆ alkoxy, C₁-C₆ haloalkoxy, C₃-C₆ cycloalkoxy, C₂-C₆ alkylcarbonyloxy, C₂-C₆ alkylcarbonyl, C₂-C₆ haloalkylcarbonyl, C₃-C₆ alkylcarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylsulfonyloxy, C₁-C₆ haloalkylsulfonyloxy, C₁-C₆ alkylamino, C₂-C₆ dialkylamino, C₂-C₈ alkylaminocarbonyl, C₃-C₁₀ dialkylaminocarbonyl, C₃-C₁₀ dihaloalkylaminocarbonyl; C₄-C₁₀ cycloalkylaminocarbonyl, C₂-C₈ haloalkylcarbonylamino, C₂-C₆ alkylcarbonylamino, —NHCH(═O), —SF₅, —SC≡N or —U—V-T; or a phenyl or phenoxy ring optionally substituted with up to 5 substituents independently selected from R²⁷;
each R^{6b} and R^{8b} is independently cyano, C₁-C₃ alkyl, C₂-C₄ alkenyl, C₂-C₄ alkynyl, C₃-C₆ cycloalkyl, C₁-C₃ alkoxy, C₂-C₄ alkoxyalkyl, C₂-C₄ alkylcarbonyl, C₂-C₄ alkoxycarbonyl, C₂-C₄ alkylaminoalkyl or C₃-C₄ dialkylaminoalkyl,
R^{7a} and R^{7b} are each H, hydroxy, amino, C₂-C₆ alkenyl, C₂-C₆ alkynyl, C₃-C₆ cycloalkyl, C(═O)H, S(═O)ₜR¹⁴, C(═Z)R¹⁵ or OR¹⁶; or C₁-C₆ alkyl or C₁-C₆ haloalkyl, each optionally substituted with up to 2 substituents independently selected from R¹⁷;
each R^{8c} is independently H, cyano, C₂-C₃ alkylcarbonyl or C₂-C₃ haloalkylcarbonyl;
R⁹ is H, CH(═O) or C₃-C₆ cycloalkyl; or C₁-C₆ alkyl, C₂-C₆ alkenyl or C₁-C₆ haloalkyl, each optionally substituted with up to 2 substituents independently selected from R¹⁹ or a phenyl ring optionally substituted with up to 5 substituents independently selected from R²⁸; or
R⁹ and R^{7a} are taken together to form a ring selected from the group consisting 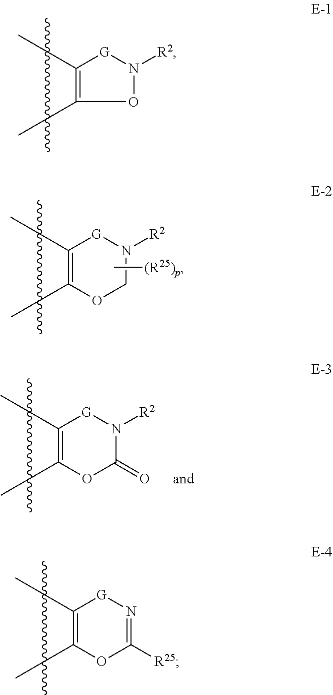
R¹⁰ is —C₁-C₆ alkyl, C₁-C₆ haloalkyl, C₂-C₆ alkenyl, C₂-C₆ haloalkenyl, C₃-C₆ cycloalkyl, C₁-C₆ alkoxy, C₁-C₆ haloalkoxy, C₂-C₆ alkoxyalkyl, C₁-C₆ alkylthio, C₁-C₆ haloalkylthio, C₂-C₆ alkylthioalkyl, C₂-C₆ alkylaminoalkyl, C₃-C₆ dialkylaminoalkyl; or a phenyl ring optionally substituted with up to 5 substituents independently selected from R²⁸;
R¹¹ is —C₁-C₆ alkyl, C₁-C₆ haloalkyl, C₂-C₆ alkenyl, C₂-C₆ haloalkenyl, C₃-C₆ cycloalkyl, C₁-C₆ alkoxy, C₁-C₆ haloalkoxy, C₂-C₆ alkoxyalkyl, C₁-C₆ alkylthio, C₁-C₆ haloalkylthio, C₂-C₆ alkylthioalkyl, C₂-C₆ alkylaminoalkyl, C₃-C₆ dialkylaminoalkyl; or phenyl or phenylmethyl, each optionally substituted on ring members with up to 5 substituents independently selected from R²⁹;
each R^{11a} is independently H, C₁-C₃ alkyl, C₁-C₃ haloalkyl or C₁-C₃ cycloalkyl;
each R^{12a} is independently H, C₁-C₃ alkyl, C₁-C₃ haloalkyl, C₂-C₃ alkylcarbonyl or C₂-C₃ haloalkylcarbonyl;
R¹² and R¹³ are each independently H, C₁-C₆ alkyl, C₁-C₆ haloalkyl, C₂-C₆ alkenyl, C₂-C₆ haloalkenyl, C₃-C₆ cycloalkyl, C₂-C₆ alkylcarbonyl, C₂-C₆ haloalkylcarbonyl, C₂-C₆ alkoxycarbonyl or C₂-C₆ haloalkoxycarbonyl;
R¹⁴ is —C₁-C₆ alkyl or C₁-C₆ haloalkyl;
R¹⁵ is C₁-C₆ alkyl, C₁-C₆ haloalkyl, C₁-C₆ alkoxy, C₁-C₆ haloalkoxy, C₂-C₆ alkoxyalkyl, C₁-C₆ alkylthio, C₂-C₆ alkylthioalkyl, C₂-C₆ alkylaminoalkyl, C₃-C₆ dialkylaminoalkyl; or a phenyl ring optionally substituted with up to 5 substituents independently selected from R³⁰;
R¹⁶ is H, CH(═O), C₃-C₆ cycloalkyl or (C═Z)R²⁰; or C₁-C₆ alkyl or C₁-C₆ haloalkyl, each optionally substituted with up to 2 substituents independently selected from R¹⁸;
each R¹⁷, R¹⁸ and R¹⁹ is independently cyano, C₃-C₆ cycloalkyl, C₁-C₆ alkoxy, C₁-C₆ haloalkoxy, C₁-C₆ alkylthio, C₁-C₆ alkylsulfinyl or C₁-C₆ alkylsulfonyl;
R²⁰ are each independently C₁-C₆ alkyl, C₁-C₆ haloalkyl, C₂-C₆ alkenyl, C₂-C₆ haloalkenyl, C₁-C₆ alkoxy, C₁-C₆ haloalkoxy, C₂-C₆ alkoxyalkyl, C₁-C₆ alkylthio, C₂-C₆ alkylthioalkyl, C₂-C₆ alkylaminoalkyl or C₃-C₆ dialkylaminoalkyl,
each U is independently O, S(═O)ₛ, N(R²¹) or a direct bond;
each V is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalkyl, C₁-C₆ alkoxy and C₁-C₆ haloalkoxy;
each T is independently cyano, NR^{22a}R^{22b}, OR²³ or S(═O)ₛR²³_{;}
each R²¹ is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{22a} and R^{22b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{22a} and R^{22b} are taken together with the nitrogen atom to which they are attached to form a form a 3- to 6-membered heterocyclic ring, the ring optionally substituted with up to 5 substituents independently selected from R²⁴;
each R²³ is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, or C₂-C₆ alkoxycarbonyl;
each R²⁴ is independently halogen, C₁-C₆ alkyl, C₁-C₆ haloalkyl or C₁-C₆ alkoxy;
each R²⁵ is independently halogen, hydroxy, amino, cyano, nitro, C₁-C₃ alkyl, C₁-C₃ haloalkyl, C₁-C₂ alkoxy or C₁-C₂ haloalkoxy;
each R²⁶, R²⁷, R²⁸, R²⁹ and R³⁰ is independently halogen, cyano, hydroxy, nitro, amino, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₄-C₆ alkylcycloalkyl, C₄-C₆ cycloalkylalkyl, C₂-C₆ cyanoalkyl, C₁-C₆ alkoxy, C₁-C₆ haloalkoxy, C₃-C₆ cycloalkoxy, C₂-C₆ alkylcarbonyloxy, C₂-C₆ alkylcarbonyl, C₂-C₆ haloalkylcarbonyl, C₃-C₆ alkylcarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylsulfonyloxy, C₁-C₆ haloalkylsulfonyloxy, C₁-C₆ alkylamino, C₂-C₆ dialkylamino, C₂-C₆ alkylcarbonylamino, CH(═O), —NHCH(═O), —SF₅, —SC≡N or —U—V-T;
R³² and R³³ are each independently selected from the group consisting of H, C₁-C₆ alkyl, and C₁-C₆ alkoxy, C₂-C₆ alkylcarbonyl, C₂-C₆ haloalkylcarbonyl and C₂-C₆ alkoxycarbonyl;
each R³⁴ is independently C₁-C₇ alkyl or C₁-C₇ alkoxy;
each a and b are independently 0, 1 or 2 in each instance of S(═O)ₐ(═NR⁸)_{b}, provided that the sum of a and b is 0, 1 or 2;
each m, p, r and s is independently 0, 1 or 2;
each n, t and u is independently 1 or 2; and
Z is O or S.

2. A compound of claim 1 wherein:
R¹ is Br, Cl or F;
W is C(═O);
R¹ is H, C₁-C₆ alkyl or -L(CR^{5a}R^{5b})ₘQ¹;
L is a direct bond;
R^{5a} and R^{5b} are H;
m is 0;
Q¹ is phenyl optionally substituted with 1 substituent selected from R^{6a};
R^{6a} is —C₁-C₆ alkyl or C₁-C₆ haloalkyl;
G is C(═O);
A is N(R^{7a});
R^{7a} is H, C₁-C₆ alkyl or C(═Z)R¹⁵;
Z is O;
R¹⁵ is C₁-C₆ alkyl, C₁-C₆ alkoxy or an unsubstituted phenyl ring;
R² is (CR^{31a}R^{31b})ᵣQ² and Q² is a phenyl ring optionally substituted with up to 3 substituents independently selected from R^{8a};
each R^{8a} is independently selected from the group consisting of halogen, C₁-C₆ alkyl, C₁-C₆ haloalkyl, cyano, —SF₅, C₁-C₆ alkoxy, C₁-C₆ haloalkoxy, C₁-C₆ haloalkylthio, C₁-C₆ halosulfonyl, and a phenyl or phenoxy ring optionally substituted with up to 3 substituents independently selected from R²⁷;
each R²⁷ is independently selected from halogen;
R³ is OR⁹, OC(═Z)R¹⁰, OS(═O)ᵤR¹¹ or halogen;
R¹⁰ is —C₁-C₆ alkyl, C₁-C₆ haloalkyl, or phenyl optionally substituted with up to 3 substituents each independently selected from R²⁸;
R¹¹ is selected from C₁-C₆ alkyl or phenyl optionally substituted with up to 3 substituents each independently selected from R²⁹.
R²⁹ is C₁-C₄ alkyl; and
R⁴ is H, halogen, C₁-C₆ alkyl, C₁-C₆ alkoxy or phenyl optionally substituted with up to 3 substituents independently selected from R²⁶.

3. A compound of claim 2 wherein R¹ is H or C₁-C₃ alkyl;
R^{7a} is H or C(═Z)R¹⁵;
R¹⁵ is C₁-C₆ alkyl;
R² is Q² is a phenyl ring substituted with up to 2 substituents each independently selected from R^{8a};
each R^{8a} is independently selected from the group consisting of F, Cl, Br, CH₃, CF₃, cyano, —SF₅, OCH₃, OCF₃, OCHF₂, OCF₂CHF₂, —SCF₃, —S(═O)₂CH₃ and a phenyl or phenoxy ring optionally substituted with up to 3 substituents each independently selected from R²⁷;
each R²⁷ is independently selected from halogen;
R³ is OR⁹; and
R⁴ is selected from the group consisting of H, Cl, CH₃, OCH₃ and unsubstituted phenyl.

4. A compound of claim 3 wherein R¹ is H or CH₃;
R^{7a} is H;
each R^{8a} is independently selected from the group consisting of F, Cl, Br, CF₃, OCH₃ and OCF₃;
R³ is OH; and
R⁴ is H.

5. A compound of claim 4 wherein R¹ is CH₃;
R² is phenyl, substituted with 1 R^{8a} substituent; and
R^{8a} is CF₃ or OCF₃.

6. A compound of Formula 1 wherein W is C(═O), G is C(═O), A is NH, R¹ is CH₃, R² is phenyl substituted with F, R³ is OH and R⁴ is H; or
W is C(═O), G is C(═O), A is NH, R¹ is CH₃, R² is phenyl substituted with Cl, R³ is OH and R⁴ is H; or
W is C(═O), G is C(═O), A is NH, R¹ is CH₃, R² is phenyl substituted with Br, R³ is OH and R⁴ is H; or
W is C(═O), G is C(═O), A is NH, R¹ is CH₃, R² is phenyl substituted with CF₃, R³ is OH and R⁴ is H; or
W is C(═O), G is C(═O), A is NH, R¹ is CH₃, R² is phenyl substituted with CHF₂, R³ is OH and R⁴ is H; or
W is C(═O), G is C(═O), A is NH, R¹ is CH₃, R² is phenyl substituted with CH₃, R³ is OH and R⁴ is H; or
W is C(═O), G is C(═O), A is NH, R¹ is CH₃, R² is phenyl substituted with OCH₃, R³ is OH and R⁴ is H; or
W is C(═O), G is C(═O), A is NH, R¹ is CH₃, R² is phenyl substituted with OCF₃, R³ is OH and R⁴ is H; or
W is C(═O), G is C(═O), A is NH, R¹ is CH₃, R² is phenyl substituted with CN, R³ is OH and R⁴ is H; or
W is C(═O), G is C(═O), A is NH, R¹ is CH₃, R² is phenyl substituted with SF₅, R³ is OH and R⁴ is H; or
W is C(═O), G is C(═O), A is NH, R¹ is CH₃, R² is phenyl substituted with OCF₃, R³ is OSO₂Me and R⁴ is H; or
W is C(═O), G is C(═O), A is NH, R¹ is CH₃, R² is phenyl substituted with OCHF₂, R³ is OSO₂Me and R⁴ is H; or
W is C(═O), G is C(═O), A is NH, R¹ is CH₃, R² is phenyl substituted with CN, R³ is OSO₂Me and R⁴ is H; or
W is C(═O), G is C(═O), A is NH, R¹ is CH₃, R² is phenyl substituted with F, R³ is OSO₂Me and R⁴ is H; or
W is C(═O), G is C(═O), A is NH, R¹ is CH₃, R² is phenyl substituted with Br, R³ is OSO₂Me and R⁴ is H; or
W is C(═O), G is C(═O), A is NH, R¹ is CH₃, R² is phenyl substituted with Cl, R³ is OSO₂Me and R⁴ is H; or
W is C(═O), G is C(═O), A is NH, R¹ is CH₃, R² is phenyl substituted with SF₅, R³ is OSO₂Me and R⁴ is H; or
W is C(═O), G is C(═O), A is NH, R¹ is CH₃, R² is phenyl substituted with CF₃, R³ is OSO₂Me and R⁴ is H; or
W is C(═O), G is C(═O), A is NH, R¹ is CH₃, R² is phenyl substituted with OCH₃, R³ is OSO₂Me and R⁴ is H; or
W is C(═O), G is C(═O), A is NH, R¹ is CH₃, R² is phenyl substituted with CH₃, R³ is OSO₂Me and R⁴ is H.

7. A compound of claim 1 which is selected from the group:
2,3-Dihydro-5-hydroxy-2-methyl-3-oxo-N-[4-(trifluoromethoxy)phenyl]-4-pyridazinecarboxamide;
2,3-Dihydro-2-methyl-5-[(methylsulfonyl)oxy]-3-oxo-N-[4-(trifluoromethoxy)phenyl]-4-pyridazinecarboxamide;
2,3-Dihydro-5-hydroxy-2-methyl-3-oxo-N-[4-(trifluoromethyl)phenyl]-4-pyridazinecarboxamide;
N-[4-(Difluoromethoxy)phenyl]-2,3-dihydro-5-hydroxy-2-methyl-3-oxo-4-pyridazinecarboxamide;
N-[2-Fluoro-4-(trifluoromethyl)phenyl]-2,3-dihydro-5-hydroxy-2-methyl-3-oxo-4-pyridazinecarboxamide;
N-(4-Bromophenyl)-2,3-dihydro-5-hydroxy-2-methyl-3-oxo-4-pyridazinecarboxamide;
[4-[[[2,3-Dihydro-5-hydroxy-2-methyl-3-oxo-4-pyridazinyl]carbonyl]amino]phenyl]pentafluorosulfur;
N-[4-(Difluoromethoxy)phenyl]-2,3-dihydro-2-methyl-5-[(methylsulfonyl)oxy]-3-oxo-4-pyridazinecarboxamide;
N-(4-cyanophenyl)-2,3-dihydro-2-methyl-5-[(methylsulfonyl)oxy]-3-oxo-4-pyridazinecarboxamide;
[4-[[[2,3-Dihydro-2-methyl-5-[(methylsulfonyl)oxy]-3-oxo-4-pyridazinyl]carbonyl]amino]phenyl]pentafluorosulfur;
2,3-Dihydro-2-methyl-5-[(methylsulfonyl)oxy]-3-oxo-N-[4-(trifluoromethyl)phenyl]-4-pyridazinecarboxamide;
5-Chloro-2,3-dihydro-2-methyl-3-oxo-N-[4-(trifluoromethoxy)phenyl]-4-pyridazinecarboxamide;
5-Bromo-2,3-dihydro-2-methyl-3-oxo-N-[4-(trifluoromethoxy)phenyl]-4-pyridazinecarboxamide; and
6-Methyl-3-[(trifluoromethoxy)phenyl]-2H-pyridazino[4,5-e]-1,3-oxazine-2,4,5(3H,6H)-trione.

8. A fungicidal composition comprising (a) a compound of claim 1; and (b) at least one other fungicide.

9. A fungicidal composition comprising (a) a compound of claim 1; and (b) at least one additional component selected from the group consisting of surfactants, solid diluents and liquid diluents.

10. A method for controlling plant diseases caused by fungal plant pathogens comprising applying to the plant or portion thereof, or to the plant seed, a fungicidally effective amount of a compound of claim 1.
